(19) United States
(12) Patent Application Publication
(10) Pub. No.: US 2025/0051840 A1
(43) Pub. Date: Feb. 13, 2025

(54) DETECTION OF GENETIC AND EPIGENETIC INFORMATION IN A SINGLE WORKFLOW

(71) Applicant: Foundation Medicine, Inc., Boston, MA (US)

(72) Inventors: Yexun WANG, San Diego, CA (US); Nicole Jacinda LAMBERT, San Diego, CA (US); Becky WONG, San Diego, CA (US); Yu ZHENG, Lexington, MA (US); Haleigh WOOD, San Diego, CA (US)

(73) Assignee: Foundation Medicine, Inc., Boston, MA (US)

(21) Appl. No.: 18/722,508

(22) PCT Filed: Dec. 28, 2022
(86) PCT No.: PCT/US2022/082476

§ 371 Date: Jun. 20, 2024

Related U.S. Application Data
(60) Provisional application No. 63/294,640, filed on Dec. 29, 2021.

(51) Int. Cl. C12Q 1/6869; C12Q 1/6855; C12Q 1/6881; C12Q 1/6886

(52) U.S. Cl. CPC C12Q 1/6869; C12Q 1/6855; C12Q 1/6881; C12Q 1/6886; C12Q 2600/154; C12Q 2600/156

(57) ABSTRACT

Provided herein are methods related to detecting genetic and epigenetic information in a single workflow, as well as methods of treatment, uses, systems, and computer readable storage media related thereto. These methods allow, e.g., for detection of genetic variants and epigenetic modifications (e.g., methylation level) in a single workflow and/or from a single sample (e.g., a DNA sample).

### CROSS-REFERENCE TO RELATED APPLICATIONS

This application claims the benefit of U.S. Provisional Application No. 63/294,640, filed Dec. 29, 2021, which is hereby incorporated by reference in its entirety.

### REFERENCE TO AN ELECTRONIC SEQUENCE LISTING

The contents of the electronic sequence listing (197102007240seqlist.xml; Size: 4,557 bytes; and Date of Creation: Dec. 22, 2022) are incorporated herein by reference in their entirety.

### FIELD

Provided herein are methods related to detecting genetic and epigenetic information in a single workflow, as well as methods of diagnosis, prognosis, monitoring, screening, and treatment, systems, and computer-readable storage media related thereto.

### BACKGROUND

Genetic mutations are a hallmark of cancer. Meanwhile, aberrant DNA methylation is a pervasive form of epigenetic change that occurs early in carcinogenesis. Rather than using genetic variation analysis alone, new approaches in precision medicine seek to characterize and detect multiple tumor derived signals to paint a holistic picture of a patient's disease state. Taking this multi-omic approach, a high resolution tumor molecular phenotype comprised of a tumor's genetic, transcriptional, and epigenetic profile can be determined.

Obtaining multi-omic signals typically requires running multiple assays on the same input material. However, limited input material, as well as the high cost and complexity of running multiple assays can make multi-omic approaches prohibitive. In the case of liquid biopsies, limited quantities of cfDNA can limit the number of assays performed. The simultaneous identification of genetic and epigenetic signals can increase sensitivity of cancer detection, e.g., in a liquid biopsy or other sample. In a diagnostic setting, simplified workflows that maximize information output are necessary.

Traditional approaches used to detect cytosine methylation leave genomic variant calling at a disadvantage. For example, in standard methylation detection methods, chemical or enzymatic treatment of DNA converts the majority of cytosine bases to uracil bases, making cytosine mutation calling prone to error. In this process, only methylated cytosine bases are protected from conversion. As such, researchers must decide whether to assess a patient's genetic sequence variants or methylation variants as the primary biomarker.

Due to the importance of analyzing cancer-associated changes in both epigenetic and genetic variation, there remains a need for improved methods and systems that provide integrated and efficient analysis of both genetic (e.g., sequence variation) and epigenetic (e.g., methylation) information in a single workflow.

All references cited herein, including patent applications and publications, are incorporated by reference in their entirety.

### SUMMARY OF THE INVENTION

The present disclosure provides, inter alia, methods of detecting genetic and epigenetic sequence information in a single workflow. These are based at least in part on the data disclosed herein demonstrating methods that provide simultaneous, base level detection of methylation and genetic variants in a single workflow. These may find use, e.g., in detection of sequence and/or methylation variants as well as detection, monitoring, screening, diagnosis, and/or prognosis of cancer, or response to cancer treatment(s).

The present disclosure describes various techniques allowing for the simultaneous detection of both genetic and epigenetic changes in a single workflow. In standard methylation methods, chemical or enzymatic treatment of DNA converts the majority of cytosines to uracil, making cytosine mutation calling error prone. Only methylated cytosines are protected from conversion. In the methods of the present disclosure, a conversion-resistant copy of the original molecule is made to maintain the genetic information. This is accomplished, e.g., by using primer extension to copy the original DNA molecules using 5-methyl cytosine (5mC) or another cytosine analog that is resistant to the particular conversion chemistry used. The original DNA molecules maintain the methylation information, and the protected strand maintains genetic information and preserves the potential to call genetic variants. Other capabilities include the potential to use an NGS index sequence to identify not only the sample, but the strand modality (methyl or genetic). Additionally, the methyl and genetic information can be paired to understand the multi-omic signature at a single molecule level.

In one aspect, provided herein is a method of detecting genetic and epigenetic information in a single workflow. In some embodiments, the method comprises: providing a plurality of first single-stranded DNA fragments; subjecting the plurality of first single-stranded DNA fragments to a round of primer extension in the presence of: (a) a primer that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands corresponding to the first single-stranded DNA fragments and a plurality of second strands that are complementary to the first strands, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion; and detecting at least a portion of the plurality of first strands and at least a portion of the plurality of second strands. In some embodiments, the method comprises: providing a plurality of first single-stranded DNA fragments; subjecting the plurality of first single-stranded DNA fragments to a round of primer extension in the presence of: (a) a primer that anneals to at least a portion of the first single-stranded DNA fragments, (b) a nucleic acid polymerase, and (c) a mixture of nucleotides comprising a cytosine analog that is resistant to TET-assisted pyridine borane treatment, thereby generating a plurality of first strands corresponding to the first single-stranded DNA fragments and a plurality of second strands that are complementary to the first strands, wherein the second strands comprise the cytosine analog that is resistant to TET-assisted pyridine borane treatment; subjecting the pluralities of the first and second strands to TET-assisted pyridine borane treatment under conditions such that any methylated cytosines if present in the first strands undergo cytosine conversion; and detecting at least a portion of the plurality of first strands and at least a portion of the plurality of second strands.

In some embodiments, the method further comprises, prior to detecting: subjecting the plurality of first strands and/or the plurality of second strands to amplification, wherein the detecting comprises detecting at least a portion of the pluralities of first and second strands and their amplification products if present. In some embodiments, the method comprises subjecting the plurality of second strands to amplification in the presence of one or more primer(s) that anneal with at least a portion of the plurality of second strands, but not with the plurality of first strands. In some embodiments, the method comprises subjecting the plurality of first strands to amplification in the presence of one or more primer(s) that anneal with at least a portion of the plurality of first strands, but not with the plurality of second strands. In some embodiments, the one or more primer(s) anneal with at least a portion of the plurality of first strands after the first strands undergo cytosine conversion, but not before the first strands undergo cytosine conversion. In some embodiments, the one or more primer(s) anneal with at least a portion of the plurality of first strands only if cytosines of the first strands do not undergo cytosine conversion. In some embodiments, the amplification occurs after cytosine conversion.

In some embodiments, the method further comprises, prior to detecting: enriching for the plurality of second strands or their amplification products. In some embodiments, the method further comprises, prior to detecting: enriching for the plurality of first strands or their amplification products. In some embodiments, the method comprises, prior to detecting: separating one or more first strands or their amplification products from one or more second strands or their amplification products. In some embodiments, the separation comprises: (a) combining one or more bait molecules with the pluralities of first and second strands, wherein the one or more bait molecules preferentially hybridize with one or more of the first strands or their amplification products, or with one or more of the second strands or their amplification products, thereby producing nucleic acid hybrids; and (b) isolating the nucleic acid hybrids. In some embodiments, the one or more bait molecules hybridize with one or more of the first strands or their amplification products but not with the plurality of second strands or their amplification products. In some embodiments, the separation occurs after the cytosine conversion treatment, and wherein the one or more bait molecules hybridize with one or more of the first strands or their amplification products if one or more cytosines of the first strands undergo cytosine conversion. In some embodiments, the one or more bait molecules hybridize with one or more of the second strands or their amplification products but not with the plurality of first strands or their amplification products. In some embodiments, the separation comprises: (a) combining one or more first bait molecules with the pluralities of first and second strands, wherein the one or more first bait molecules preferentially hybridize with one or more of the first strands or their amplification products, thereby producing first nucleic acid hybrids; isolating the first nucleic acid hybrids; combining one or more second bait molecules with the pluralities of first and second strands, wherein the one or more second bait molecules preferentially hybridize with one or more of the second strands or their amplification products, thereby producing second nucleic acid hybrids; and isolating the second nucleic acid hybrids. In some embodiments, the pluralities of first and second strands or their amplification products are detected together. In some embodiments, the pluralities of first and second strands or their amplification products are detected separately. In some embodiments, the method comprises subjecting the plurality of first single-stranded DNA fragments to two or more rounds of primer extension prior to cytosine conversion in the presence of: (a) a primer that anneals to at least a portion of the first single-stranded DNA fragments. (b) a nucleic acid polymerase, and (c) a mixture of nucleotides comprising a cytosine analog that is resistant to cytosine conversion. In some embodiments, the method further comprises attaching one or more nucleic acid adaptors to one or more of the first single-stranded DNA fragments. In some embodiments, the method further comprises attaching one or more nucleic acid adaptors to one or more of the second strands. In some embodiments, the one or more nucleic acid adaptors are attached to the first or the second strands by ligation, transposition, tailing, or template switching.

In one aspect, provided herein is a method of detecting genetic and epigenetic information in a single workflow, comprising: providing a plurality of first single-stranded DNA fragments; attaching a first adaptor nucleic acid onto at least a portion of the first single-stranded DNA fragments of the plurality; subjecting the plurality of first single-stranded DNA fragments to a round of primer extension in the presence of: (a) a primer that anneals to at least a portion of the first single-stranded DNA fragments downstream or at the first adaptor nucleic acid. (b) a nucleic acid polymerase, and (c) a mixture of nucleotides comprising a cytosine analog that is resistant to cytosine conversion, thereby generating a plurality of first strands comprising the first single-stranded DNA fragments with the first adaptor nucleic acid and a plurality of second strands comprising second single-stranded DNA fragments complementary to the first single-stranded DNA fragments and a second adaptor nucleic acid complementary to the first adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and detecting at least a portion of the plurality of first strands and at least a portion of the plurality of second strands. In some embodiments, the method comprises: providing a plurality of first single-stranded DNA fragments; attaching a first adaptor nucleic acid onto at least a portion of the first single-stranded DNA fragments of the plurality; subjecting the plurality of first single-stranded DNA fragments to a round of primer extension in the presence of: (a) a primer that anneals to at least a portion of the first single-stranded DNA fragments downstream or at the first adaptor nucleic acid, (b) a nucleic acid polymerase, and (c) a mixture of nucleotides comprising a cytosine analog that is resistant to TET-assisted pyridine borane treatment, thereby generating a plurality of first strands comprising the first single-stranded DNA fragments with the first adaptor nucleic acid and a plurality of second strands comprising second single-stranded DNA fragments complementary to the first single-stranded DNA fragments and a second adaptor nucleic acid complementary to the first adaptor nucleic acid, wherein the second strands comprise the cytosine analog that is resistant to TET-assisted pyridine borane treatment; subjecting the pluralities of the first and second strands to TET-assisted pyridine borane treatment under conditions such that any methylated cytosines if present in the first strands undergo cytosine conversion, wherein after cytosine conversion, the sequences of the first and second adaptor nucleic acids are no longer complementary; and detecting at least a portion of the plurality of first strands and at least a portion of the plurality of second strands.

In some embodiments, the first adaptor nucleic acid is attached onto 3′ end(s) of at least a portion of the first single-stranded DNA fragments of the plurality. In some embodiments, the first adaptor nucleic acid is attached onto 5′ end(s) of at least a portion of the first single-stranded DNA fragments of the plurality. In some embodiments, the first adaptor nucleic acid comprises one or more unmethylated cytosines that are converted during cytosine conversion. In some embodiments, the first adaptor nucleic acid comprises one or more methylated cytosines, and wherein the primer comprises one or more unmethylated cytosines that are converted during cytosine conversion.

In one aspect, provided herein is a method of detecting genetic and epigenetic information in a single workflow, comprising: providing a plurality of first single-stranded DNA fragments; subjecting the plurality of first single-stranded DNA fragments to a round of primer extension in the presence of: (a) a primer comprising: (i) a second adaptor nucleic acid portion that does not anneal to the first single-stranded DNA fragments and (ii) a portion that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands comprising the first single-stranded DNA fragments with a first adaptor nucleic acid complementary to the second adaptor nucleic acid and a plurality of second strands comprising second single-stranded DNA fragments complementary to the first single-stranded DNA fragments and the second adaptor nucleic acid non-complementary to the first adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and detecting at least a portion of the plurality of first strands and at least a portion of the plurality of second strands. In some embodiments, the method comprises: providing a plurality of first single-stranded DNA fragments; subjecting the plurality of first single-stranded DNA fragments to a round of primer extension in the presence of: (a) a primer comprising: (i) a second adaptor nucleic acid portion that does not anneal to the first single-stranded DNA fragments and (ii) a portion that anneals to at least a portion of the first single-stranded DNA fragments, (b) a nucleic acid polymerase, and (c) a mixture of nucleotides comprising a cytosine analog that is resistant to TET-assisted pyridine borane treatment, thereby generating a plurality of first strands comprising the first single-stranded DNA fragments with a first adaptor nucleic acid complementary to the second adaptor nucleic acid and a plurality of second strands comprising second single-stranded DNA fragments complementary to the first single-stranded DNA fragments and the second adaptor nucleic acid non-complementary to the first adaptor nucleic acid, wherein the second strands comprise the cytosine analog that is resistant to TET-assisted pyridine borane treatment; subjecting the pluralities of the first and second strands to TET-assisted pyridine borane treatment under conditions such that any methylated cytosines if present in the first strands undergo cytosine conversion, wherein after cytosine conversion, the sequences of the first and second adaptor nucleic acids are no longer complementary; and detecting at least a portion of the plurality of first strands and at least a portion of the plurality of second strands.

In some embodiments, the second adaptor nucleic acid portion of the primer is 5′ relative to the portion of the primer that anneals to the first single-stranded DNA fragments. In some embodiments, the primer comprises one or more unmethylated cytosines that become part of the second adaptor nucleic acid after primer extension and are converted during cytosine conversion.

In one aspect, provided herein is a method of detecting genetic and epigenetic information in a single workflow, comprising: providing a plurality of first single-stranded DNA fragments; subjecting the plurality of first single-stranded DNA fragments to a round of primer extension in the presence of: (a) a primer comprising: (i) a second adaptor nucleic acid portion comprising a non-complementary 5′ overhang that does not anneal to the first single-stranded DNA fragments, and (ii) a portion that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands comprising the first single-stranded DNA fragments with a first adaptor nucleic acid complementary to the portion of the second adaptor nucleic acid that anneals to the first single-stranded DNA fragments and a plurality of second strands comprising second single-stranded DNA fragments complementary to the first single-stranded DNA fragments and the second adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion; and detecting at least a portion of the plurality of first strands and at least a portion of the plurality of second strands. In one aspect, provided herein is a method of detecting genetic and epigenetic information in a single workflow, comprising: providing a plurality of first single-stranded DNA fragments; subjecting the plurality of first single-stranded DNA fragments to a round of primer extension in the presence of: (a) a primer comprising: (i) a second adaptor nucleic acid portion comprising a non-complementary 5′ overhang that does not anneal to the first single-stranded DNA fragments, and (ii) a portion that anneals to at least a portion of the first single-stranded DNA fragments, (b) a nucleic acid polymerase, and (c) a mixture of nucleotides comprising a cytosine analog that is resistant to TET-assisted pyridine borane treatment, thereby generating a plurality of first strands comprising the first single-stranded DNA fragments with a first adaptor nucleic acid complementary to the portion of the second adaptor nucleic acid that anneals to the first single-stranded DNA fragments and a plurality of second strands comprising second single-stranded DNA fragments complementary to the first single-stranded DNA fragments and the second adaptor nucleic acid, wherein the second strands comprise the cytosine analog that is resistant to TET-assisted pyridine borane treatment; subjecting the pluralities of the first and second strands to TET-assisted pyridine borane treatment under conditions such that any methylated cytosines if present in the first strands undergo cytosine conversion; and detecting at least a portion of the plurality of first strands and at least a portion of the plurality of second strands.

In some embodiments, the method further comprises demultiplexing sequence information from the first and second strands based on the first and/or second adaptor nucleic acids. In some embodiments, the method further comprises, prior to detecting: subjecting the plurality of first strands and/or the plurality of second strands to amplification, wherein the detecting comprises detecting at least a portion of the pluralities of first and second strands and their amplification products if present. In some embodiments, the method comprises subjecting the plurality of second strands to amplification in the presence of one or more primer(s) that anneal with at least a portion of the plurality of second strands, but not with the plurality of first strands. In some embodiments, the one or more primer(s) that anneal with at least a portion of the plurality of second strands anneal with at least a portion of the second adaptor nucleic acid. In some embodiments, the method comprises subjecting the plurality of first strands to amplification in the presence of one or more primer(s) that anneal with at least a portion of the plurality of first strands, but not with the plurality of second strands. In some embodiments, the one or more primer(s) that anneal with at least a portion of the plurality of first strands anneal with at least a portion of the first adaptor nucleic acid. In some embodiments, the one or more primer(s) anneal with at least a portion of the plurality of first strands after the first strands undergo cytosine conversion, but not before the first strands undergo cytosine conversion. In some embodiments, the one or more primer(s) anneal with at least a portion of the plurality of first strands only if cytosines of the first strands do not undergo cytosine conversion. In some embodiments, the method further comprises, prior to detecting: enriching for the plurality of second strands or their amplification products. In some embodiments, the method further comprises, prior to detecting: enriching for the plurality of first strands or their amplification products. In some embodiments, the method comprises, prior to detecting: separating one or more first strands or their amplification products from one or more second strands or their amplification products. In some embodiments, the separation comprises: (a) combining one or more bait molecules with the pluralities of first and second strands, wherein the one or more bait molecules preferentially hybridize with one or more of the first strands or their amplification products, or with one or more of the second strands or their amplification products, thereby producing nucleic acid hybrids; and (b) isolating the nucleic acid hybrids. In some embodiments, the one or more bait molecules hybridize with one or more of the first strands or their amplification products but not with the plurality of second strands or their amplification products. In some embodiments, the one or more bait molecules hybridize with at least a portion of the first adaptor nucleic acid. In some embodiments, the separation occurs after the cytosine conversion treatment, and wherein the one or more bait molecules hybridize with one or more of the first strands or their amplification products if one or more cytosines of the first strands undergo cytosine conversion. In some embodiments, the one or more bait molecules hybridize with one or more of the second strands or their amplification products but not with the plurality of first strands or their amplification products. In some embodiments, the one or more bait molecules hybridize with at least a portion of the second adaptor nucleic acid. In some embodiments, the separation comprises: combining one or more first bait molecules with the pluralities of first and second strands, wherein the one or more first bait molecules preferentially hybridize with one or more of the first strands or their amplification products, thereby producing first nucleic acid hybrids; isolating the first nucleic acid hybrids; combining one or more second bait molecules with the pluralities of first and second strands, wherein the one or more second bait molecules preferentially hybridize with one or more of the second strands or their amplification products, thereby producing second nucleic acid hybrids; and isolating the second nucleic acid hybrids. In some embodiments, the one or more first bait molecules hybridize with at least a portion of the first adaptor nucleic acid, and/or wherein the one or more second bait molecules hybridize with at least a portion of the second adaptor nucleic acid. In some embodiments, the pluralities of first and second strands or their amplification products are detected together. In some embodiments, the pluralities of first and second strands or their amplification products are detected separately. In some embodiments, the method comprises subjecting the plurality of first single-stranded DNA fragments to two or more rounds of primer extension prior to cytosine conversion in the presence of: (a) a primer that anneals to at least a portion of the first single-stranded DNA fragments, (b) a nucleic acid polymerase, and (c) a mixture of nucleotides comprising a cytosine analog that is resistant to cytosine conversion.

In some embodiments according to any of the embodiments described herein, the detecting is by sequencing (e.g., NGS), microarray, quantitative PCR (qPCR), digital droplet PCR (ddPCR), or molecular inversion probes. In some embodiments, the plurality of first strands is sequenced at a different depth of sequencing than the plurality of second strands. In some embodiments, the plurality of second strands is sequenced at a depth of sequencing that is at least 2×, at least 4×, at least 8×, at least 10×, at least 50×, at least 100×, or at least 1000× higher than depth of sequencing of the plurality of first strands. In some embodiments, the plurality of first strands is sequenced at a depth of sequencing that is at least 2×, at least 4×, at least 8×, at least 10×, at least 50×, at least 100×, or at least 1000× higher than depth of sequencing of the plurality of second strands. In some embodiments, after primer extension, the plurality of first strands is hybridized with the plurality of second strands in a plurality of double-stranded nucleic acids. In some embodiments, the method further comprises, prior to the round of primer extension: denaturing a plurality of double-stranded DNA fragments to provide the plurality of single-stranded DNA fragments. In some embodiments, the method further comprises obtaining the plurality of single- or double-stranded DNA fragments from a sample. In some embodiments, the method further comprises obtaining the sample from an individual. In some embodiments, the individual has, is suspected to have, or is being treated for cancer. In some embodiments, the individual is being screened for cancer or a recurrence of cancer. In some embodiments, the sample comprises tissue, cells, and/or nucleic acids from a cancer. In some embodiments, the sample comprises tissue, cells, and/or nucleic acids from normal tissue. In some embodiments, the sample comprises a tissue biopsy sample, a liquid biopsy sample, or a normal control. In some embodiments, the sample is from a tumor biopsy, tumor specimen, or circulating tumor cell. In some embodiments, the sample is a liquid biopsy sample and comprises blood, plasma, serum, cerebrospinal fluid, sputum, stool, urine, or saliva. In some embodiments, the cytosine analog that is resistant to cytosine conversion comprises 5-methylcytosine (5mC), 5-hydroxymethylcytosine (5hmC), 5-carboxylcytosine (5caC), 5-formylcytosine, 5-(beta-D-glucosylmethyl) cytosine (5gmC), 5-ethyl dCTP. 5-methyl dCTP, 5-fluoro dCTP, 5-bromo dCTP. 5-iodo dCTP, 5-chloro dCTP, 5-trifluoromethyl dCTP, or 5-aza dCTP. In some embodiments, the cytosine conversion is by bisulfite treatment, TET-assisted bisulfite treatment, oxidative bisulfite treatment, APOBEC, or TET/beta-glucosyltransferase assisted APOBEC treatment. In some embodiments, at least 80%, at least 85%, at least 90%, at least 95%, or 100% of unmethylated cytosine(s) if present in the first strands undergo cytosine conversion as a result of the cytosine conversion treatment. In some embodiments, between about 80% and about 97% of unmethylated cytosine(s) if present in the first strands undergo cytosine conversion as a result of the cytosine conversion treatment. In some embodiments, at most 20%, at most 15%, at most 10%, at most 5%, at most 2%, at most 1%, or at most 0.5% of cytosine analogs of the second strands undergo cytosine conversion as a result of the cytosine conversion treatment. In some embodiments, between about 0.5% and about 5% of cytosine analogs of the second strands undergo cytosine conversion as a result of the cytosine conversion treatment. In some embodiments, the nucleic acid polymerase is capable of incorporating the cytosine analog into nucleic acid. In some embodiments, the method further comprises, prior to primer extension: subjecting the plurality of first single-stranded DNA fragments to end repair. In some embodiments, the method further comprises, after sequencing the pluralities of first and second strands or their amplification products: comparing a sequence of the plurality of the second strands with a sequence of the plurality of the first strands. In some embodiments, the method further comprises, after sequencing the pluralities of first and second strands or their amplification products: comparing a sequence of the plurality of the first and/or the second strands with a reference genome sequence.

In one aspect, provided herein is a method of detecting cancer in an individual, comprising detecting methylation level and/or somatic mutation(s) according to the method of any one of the above embodiments in a sample comprising a plurality of nucleic acids obtained from the individual, wherein the methylation level and/or somatic mutation(s) detected in the sample identify the individual as having cancer

In one aspect, provided herein is a method of detecting minimal residual disease in an individual who has been treated or is undergoing treatment for cancer, comprising detecting methylation level and/or somatic mutation(s) according to the method of any one of the above embodiments in a sample comprising a plurality of nucleic acids obtained from the individual, wherein the methylation level and/or somatic mutation(s) detected in the sample identify the individual as having minimal residual disease or a lack thereof.

In one aspect, provided herein is a method of screening an individual suspected of having cancer, comprising detecting methylation level and/or somatic mutation(s) according to the method of any one of the above embodiments in a sample comprising a plurality of nucleic acids obtained from the individual, wherein the methylation level and/or somatic mutation(s) detected in the sample identify the individual as likely to have cancer.

In one aspect, provided herein is a method of determining prognosis of an individual having cancer, comprising detecting methylation level and/or somatic mutation(s) according to the method of any one of the above embodiments in a sample comprising a plurality of nucleic acids obtained from the individual, wherein the methylation level and/or somatic mutation(s) detected in the sample determine at least in part the prognosis of the individual.

In one aspect, provided herein is a method of predicting survival of an individual having cancer, comprising detecting methylation level and/or somatic mutation(s) according to the method of any one of the above embodiments in a sample comprising a plurality of nucleic acids obtained from the individual, wherein the methylation level and/or somatic mutation(s) detected in the sample predict at least in part the survival of the individual.

In one aspect, provided herein is a method of predicting or detecting tumor burden of an individual having cancer, comprising detecting methylation level and/or somatic mutation(s) according to the method of any one of the above embodiments in a sample comprising a plurality of nucleic acids obtained from the individual, wherein the methylation level and/or somatic mutation(s) detected in the sample predict or detect at least in part the tumor burden of the individual.

In one aspect, provided herein is a method of predicting responsiveness to treatment of an individual having cancer, comprising detecting methylation level and/or somatic mutation(s) according to the method of any one of the above embodiments in a sample comprising a plurality of nucleic acids obtained from the individual, wherein the methylation level and/or somatic mutation(s) detected in the sample are used at least in part to predict responsiveness of the individual to a treatment.

In one aspect, provided herein is a method of monitoring response of an individual being treated for cancer, comprising: administering a treatment to an individual having cancer; and detecting methylation level and/or somatic mutation(s) according to the method of any one of the above embodiments in a sample comprising a plurality of nucleic acids obtained from the individual; wherein the methylation level and/or somatic mutation(s) detected in the sample are used at least in part to monitor response to the treatment.

In one aspect, provided herein is a method of monitoring a cancer in an individual, comprising: detecting methylation level and/or somatic mutation(s) according to the method of any one of the above embodiments in a first sample comprising a plurality of nucleic acids obtained from the individual; detecting methylation level and/or somatic mutation(s) according to the method of any one of the above embodiments in a second sample comprising a plurality of nucleic acids obtained from the individual; and determining a difference in methylation level and/or somatic mutation(s) between the first and second samples, thereby monitoring the cancer in the individual.

In one aspect, provided herein is a system comprising: one or more processors; and a memory configured to store one or more computer program instructions, wherein the one or more computer program instructions when executed by the one or more processors are configured to perform the method according to any one of the above embodiments. In one aspect, provided herein is a system comprising: one or more processors; and a memory configured to store one or more computer program instructions, wherein the one or more computer program instructions when executed by the one or more processors are configured to: obtain a first plurality of sequence reads of one or more first nucleic acid molecules or their amplification products, wherein the first nucleic acid molecules have undergone cytosine conversion treatment under conditions such that unmethylated cytosine(s) if present in the first nucleic acid molecules have undergone cytosine conversion; obtain a second plurality of sequence reads of one or more second nucleic acid molecules or their amplification products, wherein the second nucleic acid molecules are complementary to the first nucleic acid molecules prior to cytosine conversion and comprise a cytosine analog that is resistant to cytosine conversion; analyze the first plurality of sequence reads for the presence or absence of methylation at one or more cytosines inferred based on cytosine conversion or a lack thereof; and analyze the second plurality of sequence reads for sequence information.

In some embodiments, the one or more first nucleic acid molecules or their amplification products further comprise a first adaptor nucleic acid sequence, and wherein the one or more computer program instructions when executed by the one or more processors are further configured to: demultiplex the first and second pluralities of sequence reads based at least in part on detection of the first adaptor nucleic acid sequence in the first plurality of sequence reads. In some embodiments, the first adaptor nucleic acid is attached onto 3′ end(s) of at least a portion of the first nucleic acid molecules of the plurality. In some embodiments, the first adaptor nucleic acid is attached onto 5′ end(s) of at least a portion of the first nucleic acid molecules of the plurality. In some embodiments, the first adaptor nucleic acid comprises one or more unmethylated cytosines that are converted during cytosine conversion. In some embodiments, the first adaptor nucleic acid comprises one or more methylated cytosines, and wherein the primer comprises one or more unmethylated cytosines that are converted during cytosine conversion. In some embodiments, the first adaptor nucleic acid is between 5′ and 3′ ends of at least a portion of the first nucleic acid molecules of the plurality. In some embodiments, the one or more second nucleic acid molecules or their amplification products further comprise a second adaptor nucleic acid sequence, and wherein the one or more computer program instructions when executed by the one or more processors are further configured to: demultiplex the first and second pluralities of sequence reads based at least in part on detection of the second adaptor nucleic acid sequence in the second plurality of sequence reads. In some embodiments, the second adaptor nucleic acid is attached onto 3′ end(s) of at least a portion of the second nucleic acid molecules of the plurality. In some embodiments, the second adaptor nucleic acid is attached onto 5′ end(s) of at least a portion of the second nucleic acid molecules of the plurality. In some embodiments, the second adaptor nucleic acid is between 5′ and 3′ ends of at least a portion of the second nucleic acid molecules of the plurality. In some embodiments, the first plurality of sequence reads is at least 2×, at least 4×, at least 8×, at least 10×, at least 50×, at least 100×, or at least 1000× more abundant than the second plurality of sequence reads. In some embodiments, the second plurality of sequence reads is at least 2×, at least 4×, at least 8×, at least 10×, at least 50×, at least 100×, or at least 1000× more abundant than the first plurality of sequence reads. In some embodiments, the first nucleic acid molecules are obtained from a sample prior to cytosine conversion. In some embodiments, the sample is from an individual having or suspected of having a cancer. In some embodiments, the sample comprises tissue, cells, and/or nucleic acids from a cancer. In some embodiments, the sample comprises tissue, cells, and/or nucleic acids from normal tissue. In some embodiments, the first and/or second plurality of sequence reads is obtained by sequencing; optionally wherein the sequencing comprises use of a massively parallel sequencing (MPS) technique, whole genome sequencing (WGS), whole exome sequencing, targeted sequencing, direct sequencing, or a Sanger sequencing technique; and optionally wherein the massively parallel sequencing technique comprises next generation sequencing (NGS). In some embodiments, the one or more program instructions when executed by the one or more processors are further configured to generate, based at least in part on the analyzing, a molecular profile for the sample. In some embodiments, the individual is administered a treatment based at least in part on the molecular profile. In some embodiments, the molecular profile further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the molecular profile further comprises results from a nucleic acid sequencing-based test. In some embodiments, the one or more computer program instructions when executed by the one or more processors are further configured to: compare a sequence read of the second plurality of sequence reads with a sequence read of the first plurality of sequence reads. In some embodiments, the one or more computer program instructions when executed by the one or more processors are further configured to: compare one or more sequence read(s) of the first and/or second plurality of sequence reads with a reference genome sequence.

In one aspect, provided herein is a non-transitory computer readable storage medium comprising one or more programs executable by one or more computer processors for performing the method according to any one of the above embodiments. In one aspect, provided herein is a non-transitory computer readable storage medium comprising one or more programs executable by one or more computer processors for performing a method comprising: obtaining a first plurality of sequence reads of one or more first nucleic acid molecules or their amplification products, wherein the first nucleic acid molecules have undergone cytosine conversion treatment under conditions such that unmethylated cytosine(s) if present in the first nucleic acid molecules have undergone cytosine conversion; obtaining a second plurality of sequence reads of one or more second nucleic acid molecules or their amplification products, wherein the second nucleic acid molecules are complementary to the first nucleic acid molecules prior to cytosine conversion and comprise a cytosine analog that is resistant to cytosine conversion; analyzing the first plurality of sequence reads for the presence or absence of methylation at one or more cytosines inferred based on cytosine conversion or a lack thereof; and analyzing the second plurality of sequence reads for sequence information.

In some embodiments, the one or more first nucleic acid molecules or their amplification products further comprise a first adaptor nucleic acid sequence, and the method further comprises: demultiplexing the first and second pluralities of sequence reads based at least in part on detection of the first adaptor nucleic acid sequence in the first plurality of sequence reads. In some embodiments, the first adaptor nucleic acid is attached onto 3′ end(s) of at least a portion of the first nucleic acid molecules of the plurality. In some embodiments, the first adaptor nucleic acid is attached onto 5′ end(s) of at least a portion of the first nucleic acid molecules of the plurality. In some embodiments, the first adaptor nucleic acid comprises one or more unmethylated cytosines that are converted during cytosine conversion. In some embodiments, the first adaptor nucleic acid comprises one or more methylated cytosines, and wherein the primer comprises one or more unmethylated cytosines that are converted during cytosine conversion. In some embodiments, the one or more second nucleic acid molecules or their amplification products further comprise a second adaptor nucleic acid sequence, and the method further comprises: demultiplexing the first and second pluralities of sequence reads based at least in part on detection of the second adaptor nucleic acid sequence in the second plurality of sequence reads. In some embodiments, the second adaptor nucleic acid is attached onto 3′ end(s) of at least a portion of the second nucleic acid molecules of the plurality. In some embodiments, the second adaptor nucleic acid is attached onto 5′ end(s) of at least a portion of the second nucleic acid molecules of the plurality. In some embodiments, the first adaptor nucleic acid is between 5′ and 3′ ends of at least a portion of the first nucleic acid molecules of the plurality. In some embodiments, the first plurality of sequence reads is at least 2×, at least 4×, at least 8×, at least 10×, at least 50×, at least 100×, or at least 1000× more abundant than the second plurality of sequence reads. In some embodiments, the second plurality of sequence reads is at least 2×, at least 4×, at least 8×, at least 10×, at least 50×, at least 100×, or at least 1000× more abundant than the first plurality of sequence reads. In some embodiments, the first nucleic acid molecules are obtained from a sample prior to cytosine conversion. In some embodiments, the sample is from an individual having or suspected of having a cancer. In some embodiments, the sample comprises tissue, cells, and/or nucleic acids from a cancer. In some embodiments, the sample comprises tissue, cells, and/or nucleic acids from normal tissue. In some embodiments, the first and/or second plurality of sequence reads is obtained by sequencing; optionally wherein the sequencing comprises use of a massively parallel sequencing (MPS) technique, whole genome sequencing (WGS), whole exome sequencing, targeted sequencing, direct sequencing, or a Sanger sequencing technique; and optionally wherein the massively parallel sequencing technique comprises next generation sequencing (NGS). In some embodiments, the method further comprises generating, based at least in part on the analyzing, a molecular profile for the sample. In some embodiments, the individual is administered a treatment based at least in part on the molecular profile. In some embodiments, the molecular profile further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the molecular profile further comprises results from a nucleic acid sequencing-based test. In some embodiments, the method further comprises comparing a sequence read of the second plurality of sequence reads with a sequence read of the first plurality of sequence reads. In some embodiments, the method further comprises comparing one or more sequence read(s) of the first and/or second plurality of sequence reads with a reference genome sequence.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 illustrates an exemplary single workflow assay to generate genomic and methylation strands, in accordance with some embodiments.

FIG. 2 illustrates an exemplary strategy to label and demultiplex genomic and methylation strands in a single workflow assay, in accordance with some embodiments. Sequences shown are CTGATCGTGGTT (SEQ ID NO:3; top) and CTGATCGTGGCC (SEQ ID NO: 4; bottom).

FIG. 3A illustrates exemplary strategies for workstream tags to label and amplify genomic and methylation strands in a single workflow assay, in accordance with some embodiments.

FIG. 3B illustrates exemplary strategies for workstream tags containing a unique sequence that distinguishes the genomic strand from the methylation strand without requiring cytosine conversion.

FIG. 3C illustrates exemplary strategies for strand-specific amplification, in accordance with some embodiments.

FIGS. 4A-4F provide flow charts for the steps of exemplary single workflow assays, in accordance with some embodiments. FIG. 4A illustrates an exemplary assay comprising end repair, adaptor ligation, primer extension, cytosine conversion treatment, PCR amplification/library generation, and sequencing the genomic and methylation strands in parallel.

FIG. 4B illustrates an exemplary assay comprising end repair, adaptor ligation, primer extension, cytosine conversion treatment, PCR amplification/library generation, hybrid-capture to enrich the genomic and methylation strands, and sequencing the genomic and methylation strands. FIG. 4C illustrates an exemplary assay comprising end repair, adaptor ligation, primer extension, cytosine conversion treatment, and PCR amplification/library generation, followed by enrichment and sequencing separate libraries for the genomic and methylation strands. FIG. 4D illustrates an exemplary assay comprising end repair, adaptor ligation, primer extension, cytosine conversion treatment, and PCR amplification/library generation, followed by enrichment of the genomic strand library and sequencing of the genomic and methylation strand libraries. FIG. 4E illustrates an exemplary assay comprising end repair, adaptor ligation, primer extension, cytosine conversion treatment, and PCR amplification/library generation, followed by enrichment of the methylation strand library and sequencing of the genomic and methylation strand libraries. FIG. 4F illustrates an exemplary assay comprising end repair, adaptor ligation, primer extension, cytosine conversion treatment, PCR amplification/library generation, hybrid-capture to enrich the genomic and methylation strands, and generation and sequencing of separate targeted libraries based on the genomic and methylation strands.

FIGS. 5A-5C show a proof-of-concept demonstration of analyzing genetic (e.g., sequence) and epigenetic (e.g., methylation) information in a single workflow. An overview of the workflow used is provided in FIG. 5A. FIG. 5B shows the percentage of sequence reads identified from the genomic and methylation strands and their amplification products. FIG. 5C shows the results of cytosine conversion: genomic strand was largely preserved after cytosine conversion, whereas the methylation strand was efficiently converted.

FIG. 6 shows protection efficiency (from cytosine conversion) observed in genomic and methylation strands using single workflow methods.

FIG. 7 shows abundance of reads identified for genomic and methylation strands after primer extension using single workflow methods.

FIG. 8 shows the correlation between cancer methylation score (assessing consensus methylated sites from individual DNA molecules) obtained using in single workflow and standard whole genome (WG) enzymatic methylation sequencing methods.

FIGS. 9A-9E show correlation of methylation levels observed using the single workflow or standard WG methylation methodologies. Methylation levels from smaller bins and functional regions were analyzed, including 1 kb bins (FIG. 9A), 10 kb bins (FIG. 9B), 100 kb bins (FIG. 9C), CpG islands (FIG. 9D), and CpG shores (FIG. 9E).

FIG. 10 shows Pearson Correlation Coefficient of average methylation fraction (AMF) in single workflow compared to the standard enzymatic methylation sequencing. Mean (r) obtained from each bin/functional region is shown.

FIG. 11 shows correlation of allele frequencies of key genetic variants observed using the single workflow or standard whole genome sequencing (WGS) methodologies.

FIG. 12 shows percentage of reads identified for the genomic and methylation strands with and without preferential amplification of genomic strand using strand-specific primers.

FIG. 13 shows percentage of reads identified for the genomic and methylation strands with and without preferential amplification of genomic strand and/or methylation strand using strand-specific primers.

FIG. 14A shows the mean unique coverage following multi-omic hybrid capture. Biotinylated capture baits targeting genomic biomarkers and methylation biomarkers were combined for simultaneous capture of single workflow libraries. High unique coverage was observed for genomic and methylated targets following hybrid capture.

FIG. 14B shows that there is high coverage uniformity across both methylated and genomic captured regions as measured by Fold-80 base penalty. In joint hybrid capture there is also a high on-target rate of both genomic and methylated regions showing efficient multi-omic strand enrichment.

FIG. 14C depicts a block diagram of an exemplary process for detecting methylation and genomic sequence information in a single workflow, in accordance with some embodiments.

FIG. 15 depicts an exemplary system, in accordance with some embodiments.

FIG. 16 depicts an exemplary device, in accordance with some embodiments.

### DETAILED DESCRIPTION

The present disclosure relates generally to detecting sequence and methylation information from nucleic acids in a single, integrated workflow.

The present disclosure demonstrates methods that provide efficient, comprehensive, and integrated analysis of sequence and methylation variants in a single workflow. The present disclosure demonstrates that both types of information can be efficiently obtained from single samples using small amounts of input DNA, and thus can be useful for a variety of applications including analysis of cancer-associated cfDNA. These methods generate a genomic strand that preserves sequence information and is resistant to cytosine conversion and a methylation strand that preserves methylation information and is susceptible to cytosine conversion. Both methylation level and sequence variant detection from the single workflow method were found to correlate with results obtained using dedicated, whole genome analysis of either sequence or methylation level using existing methods. Advantageously, the single workflow methods can be adapted to amplify and/or enrich for genomic and/or methylation strands, depending on preference or the focus of the assay.

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. De Vita et al., eds., J.B. Lippincott Company, 1993).

### II. Definitions

As used in this specification and the appended claims, the singular forms “a”, “an” and “the” include plural referents unless the context clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

The terms “cancer” and “cancerous” refer to or describe the physiological condition in mammals that is typically characterized by unregulated cell growth. Included in this definition are benign and malignant cancers.

The term “tumor,” as used herein, refers to all neoplastic cell growth and proliferation, whether malignant or benign, and all pre-cancerous and cancerous cells and tissues. The terms “cancer,” “cancerous,” and “tumor” are not mutually exclusive as referred to herein.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-. 2′-fluoro-, or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(0)S (“thioate”), P(S)S (“dithioate”), “(0)NR₂ (“amidate”), P(0)R, P(0) OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. A polynucleotide can contain one or more different types of modifications as described herein and/or multiple modifications of the same type. The preceding description applies to all polynucleotides referred to herein, including RNA and DNA.

“Oligonucleotide.” as used herein, generally refers to short, single stranded, polynucleotides that are, but not necessarily, less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “detection” includes any means of detecting, including direct and indirect detection.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as cytosine analogs resistant to cytosine conversion, intentional sequence alterations (such as sequence alterations introduced through a primer comprising a sequence that is hybridizable, but not complementary, to the template), and/or sequence errors that occur during amplification.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

The term “diagnosis” is used herein to refer to the identification or classification of a molecular or pathological state, disease or condition (e.g., cancer). For example, “diagnosis” may refer to identification of a particular type of cancer. “Diagnosis” may also refer to the classification of a particular subtype of cancer, for instance, by histopathological criteria, or by molecular features (e.g., a subtype characterized by expression of one or a combination of biomarkers (e.g., particular genes or proteins encoded by said genes), or by aberrant DNA methylation level and/or pattern).

The term “aiding diagnosis” is used herein to refer to methods that assist in making a clinical determination regarding the presence, or nature, of a particular type of symptom or condition of a disease or disorder (e.g., cancer). For example, a method of aiding diagnosis of a disease or condition (e.g., cancer) can comprise measuring certain somatic mutations or DNA methylation level and/or pattern in a biological sample from an individual.

The term “sample.” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, plasma, serum, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof. In some instances, the sample is a whole blood sample, a plasma sample, a serum sample, or a combination thereof. In some embodiments, the sample is from a tumor (e.g., a “tumor sample”), such as from a biopsy. In some embodiments, the sample is a formalin-fixed paraffin-embedded (FFPE) sample.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference sample,” “reference cell,” “reference tissue,” “control sample,” “control cell,” or “control tissue,” as used herein, refers to a sample, cell, tissue, standard, or level that is used for comparison purposes.

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

“Individual response” or “response” can be assessed using any endpoint indicating a benefit to the individual, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues; (4) inhibition (i.e. reduction, slowing down, or complete stopping) of metastasis; (5) relief, to some extent, of one or more symptoms associated with the disease or disorder (e.g., cancer); (6) increase or extension in the length of survival, including overall survival and progression free survival; and/or (7) decreased mortality at a given point of time following treatment.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and/or progression-free survival); resulting in an objective response (including a complete response or a partial response); or improving signs or symptoms of cancer.

An “effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent and in some embodiments stop) cancer cell infiltration into peripheral organs; inhibit (i.e., slow to some extent and in some embodiments stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), response rates (e.g., CR and PR), duration of response, and/or quality of life.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the patient herein is a human.

As used herein, “administering” is meant a method of giving a dosage of a compound (e.g., an antagonist) or a pharmaceutical composition (e.g., a pharmaceutical composition including an antagonist) to a subject (e.g., a patient). Administering can be by any suitable means, including parenteral, intrapulmonary, and intranasal, and, if desired for local treatment, intralesional administration. Parenteral infusions include, for example, intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

The term “package insert” is used to refer to instructions customarily included in commercial packages of therapeutic products, that contain information about the indications, usage, dosage, administration, combination therapy, contraindications, and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a probe for specifically detecting a biomarker (e.g., DNA methylation) described herein. In certain embodiments, the manufacture or kit is promoted, distributed, or sold as a unit for performing the methods described herein.

The term “methylation” is used herein to refer to presence of a methyl group at the C5 position of a cytosine nucleotide within DNA nucleic acids (unless context indicates otherwise). This term includes 5-methylcytosine (5mC) as well as cytosine nucleotides in which the methyl group is further modified, such as 5-hydroxymethylcytosine (5hmC). This term also includes DNA nucleic acids that have been subjected to chemical or enzymatic conversion of nucleotides, such as conversion that deaminates unmodified cytosines to uracil.

The term “aberrant methylation” is used herein to refer to a pattern of methylation that is not typically present in a normal tissue. For example, the term can refer to increased methylation at a site that is not normally methylated in a normal tissue, or decreased methylation at a site that is normally methylated in a normal tissue. In some embodiments, nucleic acids derived from a cancer cell (e.g., cancer nucleic acids) are characterized by aberrant methylation when their pattern and/or amount of methylation at one or more genomic loci differs from what is normally present at the corresponding locus/loci in a particular type of tissue.

The term “CpG dinucleotide” is used herein to refer to a region of 2 or more DNA bases in which a cytosine nucleotide is followed by a guanine nucleotide in the 5′->3′ direction, e.g., 5′-C-phosphate-G-3′. In many genomes, CpG dinucleotides can often be found in “clusters” or regions of DNA containing multiple CpG dinucleotides (also termed “CpG islands”). Much or most of DNA methylation in many genomes is present in CpG dinucleotides (in which the cytosine is methylated or hydroxymethylated).

### III. Methods, Systems, and Devices

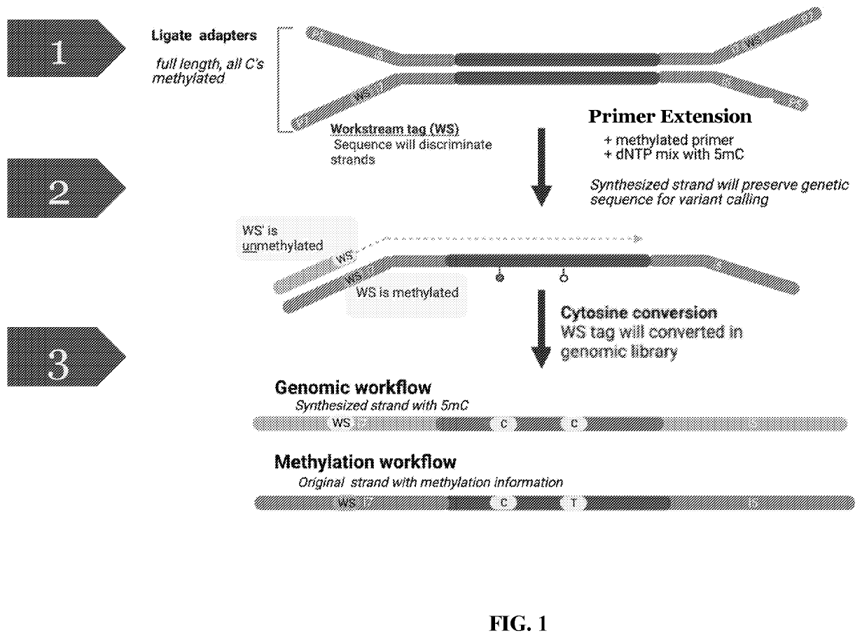

Certain aspects of the present disclosure relate to methods of detecting genetic and epigenetic information in a single workflow. In some embodiments, the methods comprise: providing a plurality of first single-stranded DNA fragments; subjecting the plurality of first single-stranded DNA fragments to a round of primer extension in the presence of: (a) a primer that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands corresponding to the first single-stranded DNA fragments and a plurality of second strands that are complementary to the first strands, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion; and detecting at least a portion of the plurality of first strands and at least a portion of the plurality of second strands. In some embodiments, detection is by microarray, quantitative PCR (qPCR), digital droplet PCR (ddPCR), molecular inversion probes, and/or sequencing. Exemplary steps for primer extension and cytosine conversion are illustrated in FIG. 1.

As discussed herein, in some embodiments, a first strand of the present disclosure (i.e., comprising methylation information, such as one or more methylated and/or unmethylated cytosines) can be referred to as a “methylation strand.” A methylation strand will contain methylation levels/patterns/marks based on the input DNA (e.g., first single-stranded DNA fragments). As discussed herein, in some embodiments, a second strand of the present disclosure (i.e., comprising a cytosine analog of the present disclosure, such as that introduced via primer extension based on a first strand of the present disclosure) and/or its amplification products can be referred to as a “genomic strand.” A genomic strand will preserve sequence information, e.g., after cytosine conversion as disclosed herein. In some embodiments, references to a “methylation strand” includes the original methylation strand and its amplification products. In some embodiments, references to a “genomic strand” includes the original genomic strand and its amplification products.

In some embodiments, the methods of the present disclosure further comprise enriching for one or both of the genomic and/or methylation strands or their amplification products. The enrichment can be accomplished, e.g., using hybrid-capture, PCR (e.g., using strand-specific primers), multiple rounds of primer extension (e.g., prior to cytosine conversion treatment), and the like. For example, in some embodiments, the methods of the present disclosure further comprise: enriching for the plurality of second strands or their amplification products, e.g., prior to detection. In some embodiments, the methods of the present disclosure further comprise: enriching for the plurality of first strands or their amplification products, e.g., prior to detection. In some embodiments, the methods of the present disclosure further comprise: enriching for the plurality of first strands or their amplification products, e.g., prior to detection; and enriching for the plurality of second strands or their amplification products, e.g., prior to detection.

In some embodiments, the method comprises, prior to detecting: separating one or more first strands or their amplification products from one or more second strands or their amplification products. In some embodiments, the separation is accomplished by hybrid-capture, e.g., using bait molecules that specifically bind/hybridize/anneal to the genomic or methylation strands or their amplification products. In some embodiments, the bait molecules specifically bind/hybridize/anneal to nucleic acid adaptor sequences, e.g., attached to the first and/or second strands.

In some embodiments, the separation comprises: (a) combining one or more bait molecules with the pluralities of first and second strands, wherein the one or more bait molecules preferentially hybridize with one or more of the first strands or their amplification products, or with one or more of the second strands or their amplification products, thereby producing nucleic acid hybrids; and (b) isolating the nucleic acid hybrids. In some embodiments, the one or more bait molecules hybridize with one or more of the first strands or their amplification products but not with the plurality of second strands or their amplification products. In some embodiments, the separation occurs after the cytosine conversion treatment, and wherein the one or more bait molecules hybridize with one or more of the first strands or their amplification products if one or more cytosines of the first strands undergo cytosine conversion. In some embodiments, the one or more bait molecules hybridize with one or more of the second strands or their amplification products but not with the plurality of first strands or their amplification products. In some embodiments, the separation comprises: combining one or more first bait molecules with the pluralities of first and second strands, wherein the one or more first bait molecules preferentially hybridize with one or more of the first strands or their amplification products, thereby producing first nucleic acid hybrids; isolating the first nucleic acid hybrids; combining one or more second bait molecules with the pluralities of first and second strands, wherein the one or more second bait molecules preferentially hybridize with one or more of the second strands or their amplification products, thereby producing second nucleic acid hybrids; isolating the second nucleic acid hybrids.

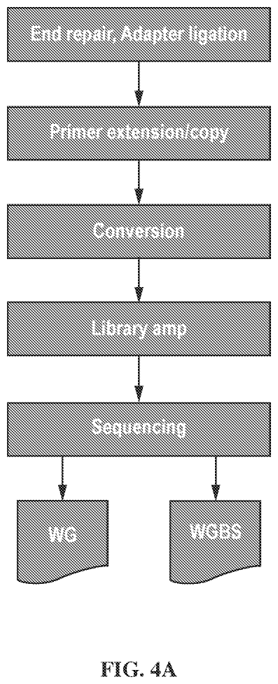

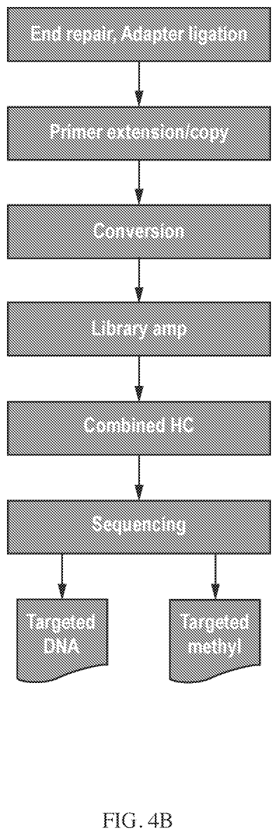

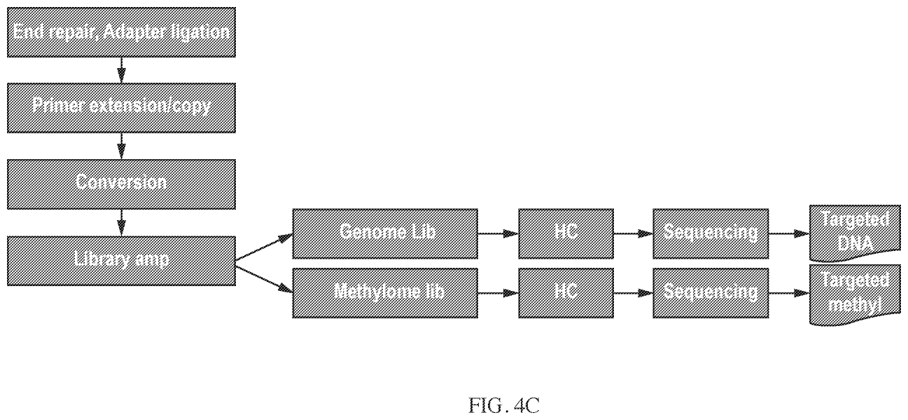

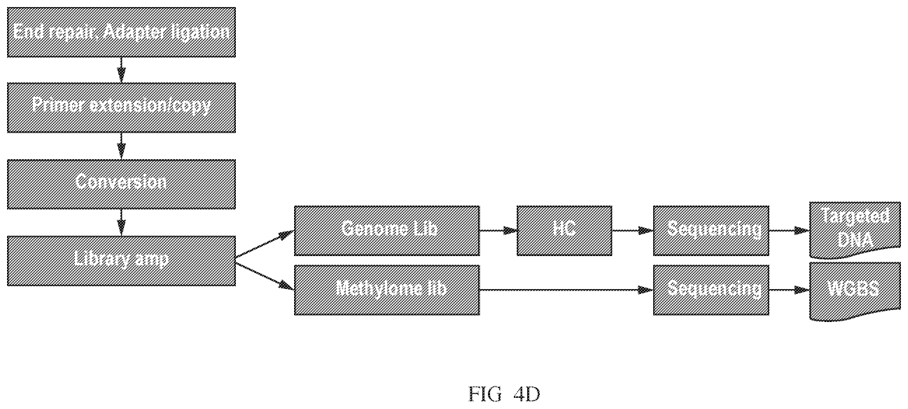

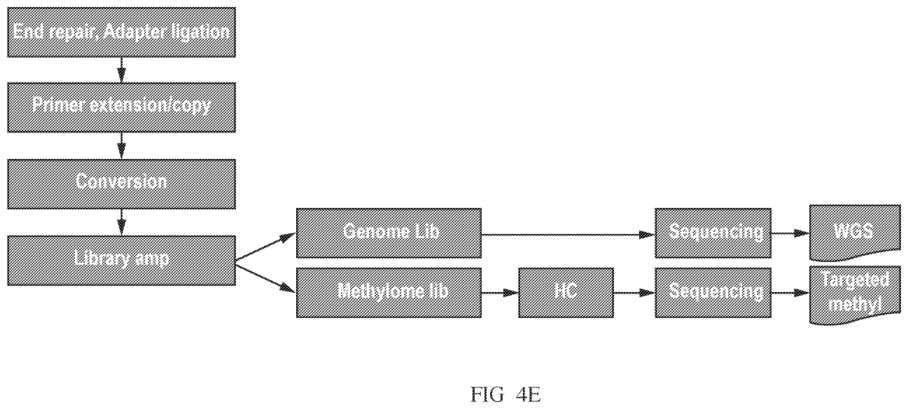

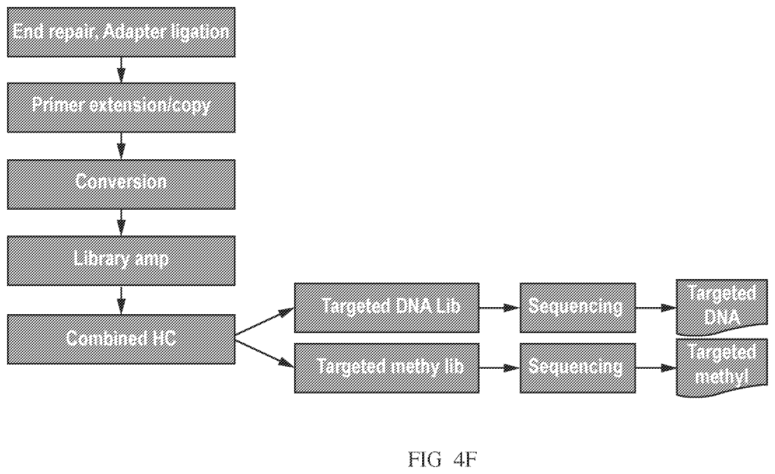

In some embodiments, the methods further comprise subjecting the plurality of first strands and/or the plurality of second strands to amplification. In some embodiments, the amplification is prior to detecting (e.g., sequencing). In some embodiments, the detecting comprises detecting at least a portion of the pluralities of first and second strands and their amplification products if present. In some embodiments, the amplification is performed after cytosine conversion treatment. See, e.g., FIGS. 4A-4F.

In some embodiments, the methods comprise subjecting the plurality of second strands to amplification in the presence of one or more primer(s) that anneal with at least a portion of the plurality of second strands, but not with the plurality of first strands. For example, the primers can be specific for the genomic strand.

In some embodiments, the methods comprise subjecting the plurality of first strands to amplification in the presence of one or more primer(s) that anneal with at least a portion of the plurality of first strands, but not with the plurality of second strands. For example, the primers can be specific for the methylation strand. In some embodiments, the one or more primer(s) anneal with at least a portion of the plurality of first strands after the first strands undergo cytosine conversion, but not before the first strands undergo cytosine conversion. In some embodiments, the one or more primer(s) anneal with at least a portion of the plurality of first strands only if cytosines of the first strands do not undergo cytosine conversion.

In some embodiments, the methods comprise subjecting the plurality of first single-stranded DNA fragments to two or more rounds of primer extension prior to cytosine conversion in the presence of: (a) a primer that anneals to at least a portion of the first single-stranded DNA fragments, (b) a nucleic acid polymerase, and (c) a mixture of nucleotides comprising a cytosine analog that is resistant to cytosine conversion. For example, multiple (instead of one) rounds of primer extension can be performed prior to cytosine conversion (using the cytosine analog) to enrich for the genomic strand.

In some embodiments, the pluralities of first and second strands or their amplification products are detected together (e.g., simultaneously) or separately.

In some embodiments, the pluralities of first and second strands or their amplification products are sequenced at the same depth of sequence. In some embodiments, the pluralities of first and second strands or their amplification products are sequenced at different depths of sequence. In some embodiments, the plurality of second strands is sequenced at a depth of sequencing that is at least 2×, at least 4×, at least 8×, at least 10×, at least 50×, at least 100×, or at least 1000× higher than depth of sequencing of the plurality of first strands. In some embodiments, the genomic strands or their amplification products are sequenced at a depth of sequencing that is at least 2×, at least 4×, at least 8×, at least 10×, at least 50×, at least 100×, or at least 1000× higher than depth of sequencing of methylation strands. In some embodiments, the plurality of first strands is sequenced at a depth of sequencing that is at least 2×, at least 4×, at least 8×, at least 10×, at least 50×, at least 100×, or at least 1000× higher than depth of sequencing of the plurality of second strands. In some embodiments, the methylation strands or their amplification products are sequenced at a depth of sequencing that is at least 2×, at least 4×, at least 8×, at least 10×, at least 50×, at least 100×, or at least 1000× higher than depth of sequencing of genomic strands. Using different sequencing depth can be advantageous depending on the application. For example, lower confidence calls such as single nucleotide polymorphisms may be subjected to higher depth of sequencing to increase confidence.

In some embodiments, after primer extension, the plurality of first strands is hybridized with the plurality of second strands in a plurality of double-stranded nucleic acids.

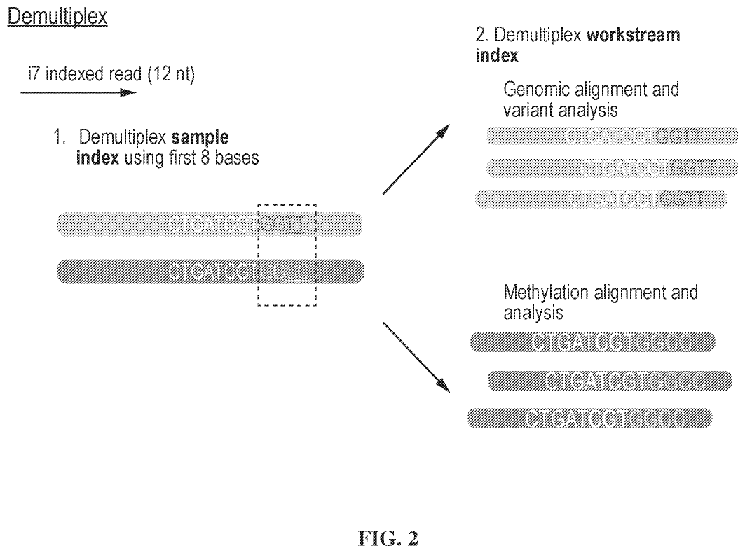

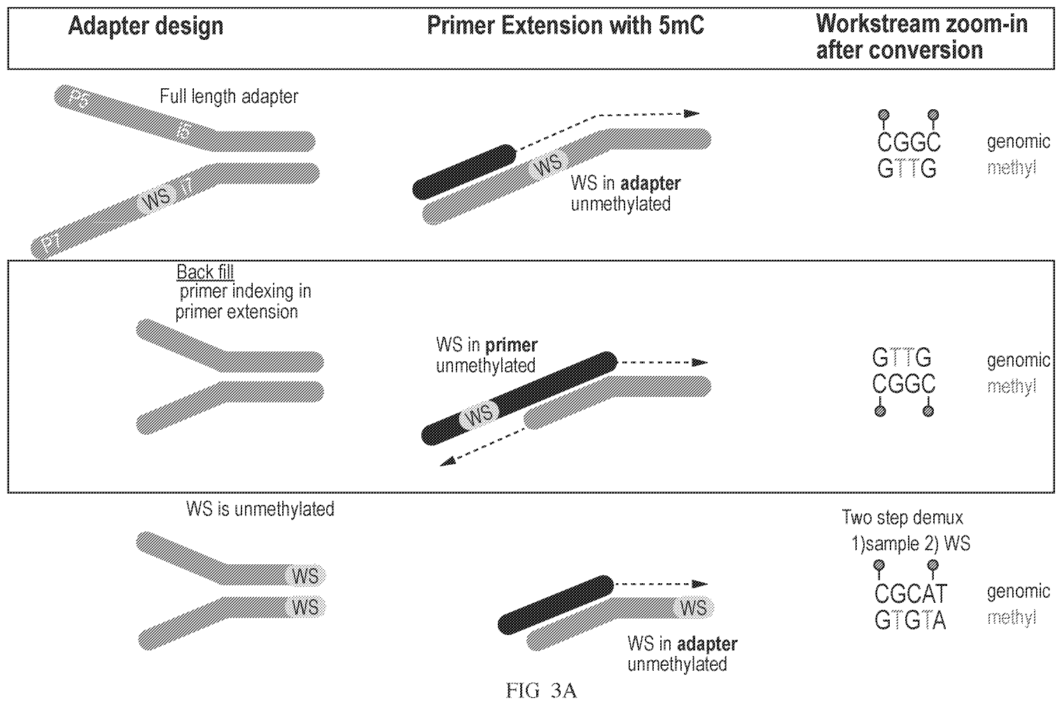

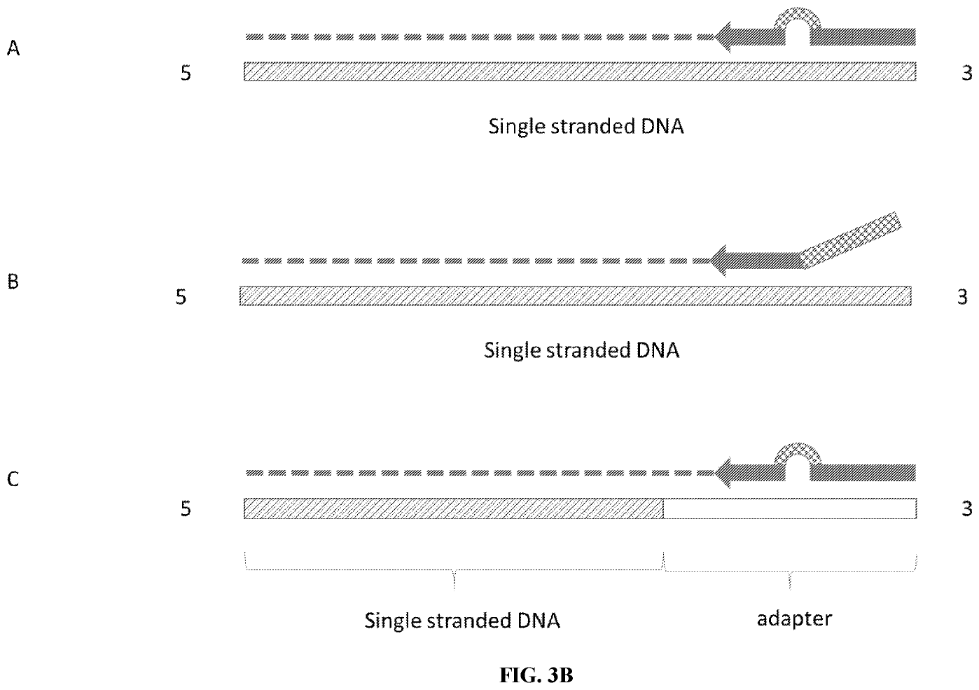

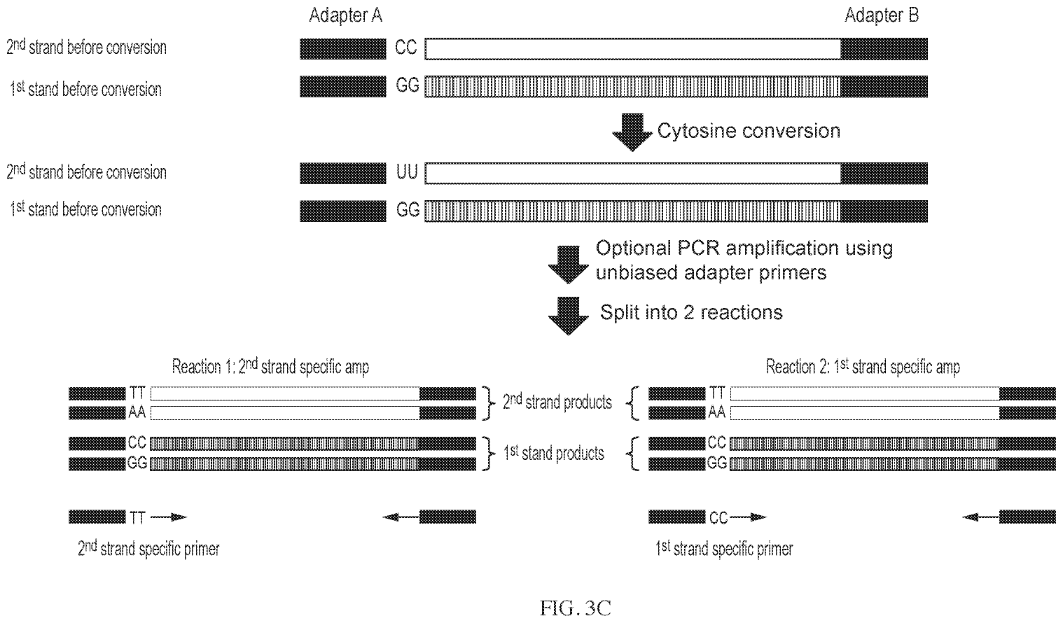

Certain aspects of the present disclosure relate to adaptor nucleic acid sequences. In some embodiments, the adaptor nucleic acid sequences can be used, e.g., to distinguish genomic vs. methylation strands or their amplification products. In some embodiments, the methods of the present disclosure comprise demultiplexing sequence information from the first and/or second strands. In some embodiments, the methods of the present disclosure comprise demultiplexing sequence information in order to distinguish sequence reads from the first or methylation strands vs. sequence reads from the second or genomic strands. In some embodiments, the demultiplexing is based at least in part on first and/or second adaptor nucleic acids of the present disclosure. In some embodiments, the adaptor nucleic acid sequences can further include sequence to encode other types of information, including but not limited to sequence(s) for identification of the sample, lab, physician, date, sequencing run, equipment, replicate, etc. In some embodiments, after cytosine conversion, adaptor nucleic acids attached to the genomic strand are no longer complementary to adaptor nucleic acids attached to the methylation strand; see, e.g., FIG. 2. In some embodiments, adaptor nucleic acids (e.g., attached to the methylation or genomic strand) comprise cytosine(s) that are methylated or unmethylated; see, e.g., FIG. 3A. In some embodiments, the adaptor nucleic acids comprise a non-complementary 5′ overhang, such that after primer extension, adaptor nucleic acids attached to the genomic strand are not complementary to adaptor nucleic acids attached to the methylation strand, even without cytosine conversion. For example, FIG. 3B shows how an adaptor nucleic acid sequence can comprise a region of non-complementarity sandwiched between two complementary portions of the single-stranded DNA (see FIG. 3B at A) or adaptor sequence (see FIG. 3B at C), or a non-complementary 5′ overhang (see FIG. 3B at B), such that after primer extension, a unique sequence in the adaptor nucleic acid distinguishing the two strands is generated independent of cytosine conversion.

In some embodiments, the methods of the present disclosure comprise selective amplification of the methylation and/or genomic strands. As shown in FIG. 3C, it is contemplated herein that strand-specific primers can be used to amplify the methylation and/or genomic strand(s) selectively, e.g., using adaptor nucleic acid sequences as primer anchors. In the exemplary methods illustrated in FIG. 3C, one or more rounds of PCR amplification are conducted after cytosine conversion (e.g., using unbiased primers), after which the methylation and genomic strands and their amplification products are amplified separately using strand-specific primers. Without wishing to be bound to theory, it is thought that preferential amplification conducted on the first and second strand products, e.g. after a universal amplification, can prevent loss of genomic strand content when amplifying methylation strands, and vice versa. If preferential amplification were instead conducted on the original strands, one strand could be diluted and information content lost when specifically amplifying the other strand.

In some embodiments, the methods of the present disclosure comprise attaching one or more nucleic acid adaptors to one or more of the first and/or second single-stranded DNA fragments. In some embodiments, the adaptors are attached via ligation, transposition, tailing, template switching, or the like.

In some embodiments, the methods comprise: providing a plurality of first single-stranded DNA fragments; subjecting the plurality of first single-stranded DNA fragments to a round of primer extension in the presence of: (a) a primer comprising: (i) a second adaptor nucleic acid portion that does not anneal to the first single-stranded DNA fragments and (ii) a portion that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands comprising the first single-stranded DNA fragments with a first adaptor nucleic acid complementary to the second adaptor nucleic acid and a plurality of second strands comprising second single-stranded DNA fragments complementary to the first single-stranded DNA fragments and the second adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and detecting at least a portion of the plurality of first strands and at least a portion of the plurality of second strands. In some embodiments, detection is by microarray, quantitative PCR (qPCR), digital droplet PCR (ddPCR), molecular inversion probes, and/or sequencing.

In some embodiments, the second adaptor nucleic acid portion of the primer is between two portions of the primer that anneal to the first single-stranded DNA fragments. In some embodiments, the second adaptor nucleic acid portion of the primer is 5′ relative to the portion of the primer that anneals to the first single-stranded DNA fragments. For example, in some embodiments, the second adaptor nucleic acid portion does not anneal to the first single-stranded DNA fragments. Thus, the second adaptor nucleic acid portion can be introduced via primer, and complementary sequence can be introduced via backfill from the 5′ end of the first single-stranded DNA fragments (see, e.g., FIG. 3, middle row). In some embodiments, the primer comprises one or more unmethylated cytosines that become part of the second adaptor nucleic acid after primer extension and are converted during cytosine conversion.

In some embodiments, the methods comprise: providing a plurality of first single-stranded DNA fragments; attaching a first adaptor nucleic acid onto at least a portion of the first single-stranded DNA fragments of the plurality; subjecting the plurality of first single-stranded DNA fragments to a round of primer extension in the presence of: (a) a primer that anneals to at least a portion of the first single-stranded DNA fragments downstream or at the first adaptor nucleic acid, (b) a nucleic acid polymerase, and (c) a mixture of nucleotides comprising a cytosine analog that is resistant to cytosine conversion, thereby generating a plurality of first strands comprising the first single-stranded DNA fragments with the first adaptor nucleic acid and a plurality of second strands comprising second single-stranded DNA fragments complementary to the first single-stranded DNA fragments and a second adaptor nucleic acid complementary to the first adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and detecting at least a portion of the plurality of first strands and at least a portion of the plurality of second strands. In some embodiments, detection is by microarray, quantitative PCR (qPCR), digital droplet PCR (ddPCR), molecular inversion probes, and/or sequencing.

In some embodiments, the first adaptor nucleic acid is attached onto 3′ end(s) of at least a portion of the first single-stranded DNA fragments of the plurality. In some embodiments, the first adaptor nucleic acid is attached onto 5′ end(s) of at least a portion of the first single-stranded DNA fragments of the plurality. In some embodiments, the first adaptor nucleic acid comprises one or more unmethylated cytosines that are converted during cytosine conversion. In some embodiments, the first adaptor nucleic acid comprises one or more methylated cytosines, and wherein the primer comprises one or more unmethylated cytosines that are converted during cytosine conversion.

In some embodiments, the methods comprise: providing a plurality of first single-stranded DNA fragments; subjecting the plurality of first single-stranded DNA fragments to a round of primer extension in the presence of: (a) a primer comprising: (i) a second adaptor nucleic acid portion comprising a non-complementary 5′ overhang that does not anneal to the first single-stranded DNA fragments, and (ii) a portion that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands comprising the first single-stranded DNA fragments with a first adaptor nucleic acid complementary to the portion of the second adaptor nucleic acid that anneals to the first single-stranded DNA fragments and a plurality of second strands comprising second single-stranded DNA fragments complementary to the first single-stranded DNA fragments and the second adaptor nucleic acid non-complementary to the first adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion; and detecting at least a portion of the plurality of first strands and at least a portion of the plurality of second strands.

In some embodiments, the methods of the present disclosure comprise end repair, adaptor ligation, primer extension, cytosine conversion treatment, PCR amplification/library generation, and sequencing the genomic and methylation strands in parallel, e.g., as described herein. See, e.g., FIG. 4A.

In some embodiments, the methods of the present disclosure comprise end repair, adaptor ligation, primer extension, cytosine conversion treatment, PCR amplification/library generation, hybrid-capture to enrich the genomic and methylation strands, and sequencing the genomic and methylation strands, e.g., as described herein. See, e.g., FIG. 4B.

In some embodiments, the methods of the present disclosure comprise end repair, adaptor ligation, primer extension, cytosine conversion treatment, and PCR amplification/library generation, e.g., as described herein. In some embodiments, separate libraries are constructed for the genomic and methylation strands. In some embodiments, each library can be enriched (e.g., via hybrid capture) and sequenced. See, e.g., FIG. 4C.

In some embodiments, the methods of the present disclosure comprise end repair, adaptor ligation, primer extension, cytosine conversion treatment, and PCR amplification/library generation, e.g., as described herein. In some embodiments, separate libraries are constructed for the genomic and methylation strands. In some embodiments, only the genomic strand library is enriched (e.g., via hybrid capture), and both libraries sequenced. See, e.g., FIG. 4D.

In some embodiments, the methods of the present disclosure comprise end repair, adaptor ligation, primer extension, cytosine conversion treatment, and PCR amplification/library generation, e.g., as described herein. In some embodiments, separate libraries are constructed for the genomic and methylation strands. In some embodiments, only the methylation strand library is enriched (e.g., via hybrid capture), and both libraries sequenced. See, e.g., FIG. 4E.

In some embodiments, the methods of the present disclosure comprise end repair, adaptor ligation, primer extension, cytosine conversion treatment, PCR amplification/library generation, hybrid-capture to enrich the genomic and methylation strands, and generation of separate libraries based on the genomic and methylation strands, e.g., as described herein. Both libraries can then be sequenced. See, e.g., FIG. 4F.

In some embodiments, single-stranded DNA fragments of the present disclosure are obtained from double-stranded DNA, e.g., from a sample of the present disclosure. In some embodiments, the methods comprise denaturing a plurality of double-stranded DNA fragments to provide the plurality of first single-stranded DNA fragments, e.g., prior to primer extension.

In some embodiments, the methods of the present disclosure are used to detect methylation of one of more CpG sites, islands, or shores. CpG dinucleotides or sites typically refer to regions of DNA where a cytosine nucleotide is located immediately adjacent to a guanine nucleotide in the linear sequence. “CpG” refers to cytosine and guanine separated by a phosphate (i.e., -C-phosphate-G-). Regions of the DNA that have a higher frequency or concentration of CpG sites are known as “CpG islands”. Many genes in mammalian genomes have CpG islands associated with the transcriptional start site (including the promoter) of the gene, which play a pivotal role in controlling gene expression. See, e.g., US PG Pub. No. US20140357497. Aberrant methylation patterns are observed in many types of cancer. For example, in normal tissue, CpG islands are often unmethylated but a subset of islands becomes methylated during oncogenesis, cellular development, and various disease states. Hypermethylation (i.e. an increased level of methylation) of CpG sites within the promoters of genes can lead to their silencing, a feature found, e.g., in a number of human cancers (for example the silencing of tumor suppressor genes).

In some embodiments, the methods of the present disclosure comprise subjecting nucleic acids (e.g., pluralities of first and second strands) to a cytosine conversion treatment, e.g., under conditions such that unmethylated cytosine(s) if present in the first strands undergo cytosine conversion (e.g., to uracil, which is converted to thymine during PCR amplification/primer extension). In some embodiments, the cytosine conversion is by bisulfite treatment, TET-assisted bisulfite treatment, oxidative bisulfite treatment, APOBEC, or TET/beta-glucosyltransferase assisted APOBEC treatment.

In some embodiments, cytosine conversion treatment converts unmethylated cytosine(s) if present in the first strands at a particular efficiency. For example, in some embodiments, at least 80%, at least 85%, at least 90%, at least 95%, or 100% of unmethylated cytosine(s) if present in the first strands undergo cytosine conversion as a result of the cytosine conversion treatment. In some embodiments, unmethylated cytosine(s) if present in the first strands undergo cytosine conversion as a result of the cytosine conversion treatment at a percentage having an upper limit of 100%, 99%, 98%, 97%, 96%, 95%, 94%, 93%, 92%, 91%, 90%, 89%, 88%, 87%, 86%, 85%, 84%, 83%, 82%, or 81% and an independently selected lower limit of 80%, 81%, 82%, 83%, 84%, 85%, 86%, 87%, 88%, 89%, 90%, 91%, 92%, 93%, 94%, 95%, 96%, 97%, or 98%, wherein the upper limit is higher than the lower limit. In some embodiments, between about 80% and about 97%, between about 85% and about 97%, between about 90% and about 97%, between about 95% and about 97%, between about 80% and about 95%, between about 85% and about 95%, or between about 90% and about 95% of unmethylated cytosine(s) if present in the first strands undergo cytosine conversion as a result of the cytosine conversion treatment. The present disclosure demonstrates infra that unmethylated cytosine(s) if present in the first strands undergo cytosine conversion at a very high efficiency.

In some embodiments, cytosine analogs of the present disclosure in the second strands are protected from cytosine conversion treatment at a particular efficiency. For example, in some embodiments, at most 20%, at most 15%, at most 10%, at most 5%, at most 2%, at most 1%, at most 0.5%, or at most 0.1% of cytosine analogs of the second strands undergo cytosine conversion as a result of the cytosine conversion treatment. In some embodiments, cytosine analogs of the second strands undergo cytosine conversion undergo cytosine conversion as a result of the cytosine conversion treatment at a percentage having an upper limit of 20%, 19%, 18%, 17%, 16%, 15%, 14%, 13%, 12%, 11%, 10%, 9%, 8%, 7%, 6%, 5%, 4%, 3%, 2%, or 1% and an independently selected lower limit of 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, or 18%, wherein the upper limit is higher than the lower limit. In some embodiments, between about 0.5% and about 5%, between about 0.5% and about 3, between about 0.5% and about 1%, between about 1% and about 5%, between about 1% and about 3%, or between about 1% and about 2% of cytosine analogs of the second strands undergo cytosine conversion as a result of the cytosine conversion treatment. The present disclosure demonstrates infra that cytosine analogs in the second strands undergo cytosine conversion at a very low efficiency.

A commonly-used method of determining the methylation level and/or pattern of DNA requires methylation status-dependent conversion of cytosine in order to distinguish between methylated and non-methylated CpG dinucleotide sequences. For example, methylation of CpG dinucleotide sequences can be measured by employing cytosine conversion based technologies, which rely on methylation status-dependent chemical modification of CpG sequences within isolated genomic DNA, or fragments thereof, followed by DNA sequence analysis. Chemical reagents that are able to distinguish between methylated and non-methylated CpG dinucleotide sequences include hydrazine, which cleaves the nucleic acid, and bisulfite treatment. Bisulfite treatment followed by alkaline hydrolysis specifically converts non-methylated cytosine to uracil, leaving 5-methylcytosine unmodified as described by Olek A., Nucleic Acids Res. 24:5064-6, 1996 or Frommer et al., Proc. Natl. Acad. Sci. USA 89:1827-1831 (1992). The bisulfite-treated DNA can subsequently be analyzed by conventional molecular techniques, such as PCR amplification, sequencing, and detection comprising oligonucleotide hybridization. See, e.g., U.S. Pat. No. 10,174,372.

Various methodologies for cytosine conversion are known in the art. In some embodiments, a plurality of nucleic acids or nucleic acid fragments of the present disclosure has undergone cytosine conversion by bisulfite treatment, TET-assisted bisulfite treatment, TET-assisted pyridine borane treatment, oxidative bisulfite treatment, or APOBEC treatment, e.g., prior to detection.

As such, in some embodiments, the methods of the present disclosure comprise treating a plurality of nucleic acids or nucleic acid fragments of the present disclosure with bisulfite. Bisulfite sequencing is a commonly used method in the art for generating methylation data at single-base resolution. Bisulfite conversion or treatment refers to a biochemical process for converting unmethylated cytosine residue to uracil or thymine residues (e.g., deamination to uracil, followed by amplification as thymine during PCR), whereby methylated cytosine residues (e.g., 5-methylcytosine, 5mC; or 5-hydroxymethylcytosine, 5hmC) are preserved. Reagents to convert cytosine to uracil are known to those of skill in the art and include bisulfite reagents such as sodium bisulfite, potassium bisulfite, ammonium bisulfite, magnesium bisulfite, sodium metabisulfite, potassium metabisulfite, ammonium metabisulfite, magnesium metabisulfite and the like.

In some embodiments, the methods of the present disclosure comprise treating a plurality of nucleic acids or nucleic acid fragments of the present disclosure with enzymatic digestion and bisulfite treatment. The principle of the method is that the fragmentation of DNA is not achieved by ultrasound but achieved by combined enzymatic digestion by multiple endonucleases (MseI, Tsp 5091, NlaIII and Hpy CH4V), wherein the restriction enzyme cutting sites of MseI, Tsp5091, NlaIII and Hpy CH4V are TTAA, AATT, CATG and TGCA, respectively. See, e.g., Smiraglia D J, et al. Oncogene 2002; 21:5414-5426. This is followed by bisulfite treatment, e.g., as described herein.

Enzymatic methods for cytosine conversion are also known, e.g., enzymatic methyl sequencing. Such approaches can be advantageous because they employ enzymes instead of bisulfite, which can damage and fragment DNA, leading to DNA loss and potentially biased sequencing. For example, TET2 (the Ten-eleven translocation (Tet) family 2 methylcytosine dioxygenase) and T4-BGT (T4 phage beta-glucosyltransferase) can be used to convert 5mC and 5hmC into products that cannot be deaminated by APOBEC3A (apolipoprotein B mRNA editing enzyme, catalytic polypeptide-like 3A), then APOBEC3A is used to deaminate unmodified cytosines by converting them into uracils. See, e.g., Vaisvila, R. et al. (2021) Genome Res. 31:1-10.

In some embodiments, the methods of the present disclosure comprise treating a plurality of nucleic acids or nucleic acid fragments of the present disclosure with TET-assisted bisulfite (e.g., TAB-seq). In the TAB-seq approach, beta-glucosyltransferase (BGT) is used to convert 5hmC into β-glucosyl-5-hydroxymethylcytosine (5gmC), and a Tet enzyme (e.g., mTet1) is used to oxidize 5mC into 5-carboxylcytosine (5caC). Subsequently, nucleic acids can be treated with bisulfite. See, e.g., Yu, M. et al. (2018) Methods Mol. Biol. 1708:645-663.

In some embodiments, the methods of the present disclosure comprise treating a plurality of nucleic acids or nucleic acid fragments of the present disclosure with TET-assisted pyridine borane (e.g., TAPS). In the TAPS approach, a TET methylcytosine dioxygenase is used to oxidize 5mC and 5hmC into 5caC, then 5caC is reduced into dihydrouracil (DHU) via pyridine borane. DHU is converted to thymine during subsequent PCR. See, e.g., Liu, Y. et al. (2019) Nat. Biotechnol. 37:424-429.

In some embodiments, the methods of the present disclosure comprise treating a plurality of nucleic acids or nucleic acid fragments of the present disclosure with oxidative bisulfite (e.g., oxBS). In the oxBS approach, 5hmC is oxidized into 5-formylcytosine (5fC), which can be converted to uracil under bisulfite. Sequencing results from bisulfite vs. oxidative bisulfite treatment can then be used to infer 5hmC levels from 5mC. See, e.g., Booth, M. J. et al. (2013) Nat. Protocols 8:1841-1851. This approach can be scaled on a genome-wide level in oxBS-seq; see, e.g., Kirschner, K. et al. (2018) Methods Mol. Biol. 1708:665-678.

In some embodiments, the methods of the present disclosure comprise treating a plurality of nucleic acids or nucleic acid fragments of the present disclosure with APOBEC. Enzymatic reagents to convert cytosine to uracil, i.e. cytosine deaminases, include those of the APOBEC family, such as APOBEC-seq or APOBEC3A. The APOBEC family members are cytidine deaminases that convert cytosine to uracil while maintaining 5-methyl cytosine, i.e. without altering 5-methyl cytosine. Such enzymes are described in US2013/0244237 and WO2018165366 and are commercially available (see, e.g., the NEBNext® Enzymatic Methyl-seq Kit, New England Biolabs). Non-limiting examples of APOBEC family proteins include APOBEC1, APOBEC2, APOBEC3A, APOBEC3B, APOBEC3C, APOBEC3D, APOBEC3F, APOBEC3G, APOBEC3H, APOBEC4, and Activation-induced (cytidine) deaminase.

In some embodiments, the methods of the present disclosure comprise one or more rounds of primer extension using a mixture of nucleotides comprising a cytosine analog that is resistant to cytosine conversion. In some embodiments, the primer extension generates a plurality of first strands corresponding to the first single-stranded DNA fragments and a plurality of second strands that are complementary to the first strands, wherein the second strands comprise the cytosine analog that is resistant to cytosine conversion. In some embodiments, the genomic strand(s) and optionally their amplification products comprise the cytosine analog. In some embodiments, the mixture of nucleotides comprises adenine, guanine, thymine/uracil, and the cytosine analog.

A variety of cytosine analogs are contemplated for use in the methods described herein. The type of cytosine analog may depend upon the type of cytosine conversion treatment, i.e., to ensure that the cytosine analog is resistant to treatment/conversion. In some embodiments, the cytosine analog that is resistant to cytosine conversion comprises 5-methylcytosine (5mC), 5-hydroxymethylcytosine (5hmC), 5-carboxylcytosine (5caC), 5-formylcytosine, 5-(beta-D-glucosylmethyl) cytosine (5gmC), 5-ethyl dCTP. 5-methyl dCTP, 5-fluoro dCTP, 5-bromo dCTP, 5-iodo dCTP, 5-chloro dCTP, 5-trifluoromethyl dCTP, or 5-aza dCTP. In some embodiments, the cytosine analog that is resistant to cytosine conversion comprises cytosine, e.g., for TET-assisted pyridine borane treatment.

In some embodiments, the polymerase used for primer extension is capable of incorporating the cytosine analog into nucleic acid.

In some embodiments, the methods further comprise subjecting the plurality of first single-stranded DNA fragments to end repair, e.g., prior to primer extension.

In some embodiments, the methods comprise use of TET-assisted pyridine borane sequencing (TAPS) treatment. As is known in the art, TET-assisted pyridine borane treatment converts 5mC and 5hmC to uracil (which can be converted to thymine during PCR amplification). As such, the methylated cytosines are converted. Therefore, it is contemplated that the methods disclosed herein can be adapted to TAPS applications by simply reversing which cytosines are converted (i.e., methylated vs. unmethylated).

In some embodiments, the methods comprise: providing a plurality of first single-stranded DNA fragments; subjecting the plurality of first single-stranded DNA fragments to a round of primer extension in the presence of: (a) a primer that anneals to at least a portion of the first single-stranded DNA fragments, (b) a nucleic acid polymerase, and (c) a mixture of nucleotides comprising a cytosine analog that is resistant to TET-assisted pyridine borane treatment, thereby generating a plurality of first strands corresponding to the first single-stranded DNA fragments and a plurality of second strands that are complementary to the first strands, wherein the second strands comprise the cytosine analog that is resistant to TET-assisted pyridine borane treatment; subjecting the pluralities of the first and second strands to TET-assisted pyridine borane treatment under conditions such that any methylated cytosines if present in the first strands undergo cytosine conversion; and detecting at least a portion of the plurality of first strands and at least a portion of the plurality of second strands. In some embodiments, the methods comprise: providing a plurality of first single-stranded DNA fragments; attaching a first adaptor nucleic acid onto at least a portion of the first single-stranded DNA fragments of the plurality; subjecting the plurality of first single-stranded DNA fragments to a round of primer extension in the presence of: (a) a primer that anneals to at least a portion of the first single-stranded DNA fragments downstream or at the first adaptor nucleic acid, (b) a nucleic acid polymerase, and (c) a mixture of nucleotides comprising a cytosine analog that is resistant to TET-assisted pyridine borane treatment, thereby generating a plurality of first strands comprising the first single-stranded DNA fragments with the first adaptor nucleic acid and a plurality of second strands comprising second single-stranded DNA fragments complementary to the first single-stranded DNA fragments and a second adaptor nucleic acid complementary to the first adaptor nucleic acid, wherein the second strands comprise the cytosine analog that is resistant to TET-assisted pyridine borane treatment; subjecting the pluralities of the first and second strands to TET-assisted pyridine borane treatment under conditions such that any methylated cytosines if present in the first strands undergo cytosine conversion, wherein after cytosine conversion, the sequences of the first and second adaptor nucleic acids are no longer complementary; and detecting at least a portion of the plurality of first strands and at least a portion of the plurality of second strands. In some embodiments, the methods comprise: providing a plurality of first single-stranded DNA fragments; subjecting the plurality of first single-stranded DNA fragments to a round of primer extension in the presence of: (a) a primer comprising: (i) a second adaptor nucleic acid portion that does not anneal to the first single-stranded DNA fragments and (ii) a portion that anneals to at least a portion of the first single-stranded DNA fragments, (b) a nucleic acid polymerase, and (c) a mixture of nucleotides comprising a cytosine analog that is resistant to TET-assisted pyridine borane treatment, thereby generating a plurality of first strands comprising the first single-stranded DNA fragments with a first adaptor nucleic acid complementary to the second adaptor nucleic acid and a plurality of second strands comprising second single-stranded DNA fragments complementary to the first single-stranded DNA fragments and the second adaptor nucleic acid non-complementary to the first adaptor nucleic acid, wherein the second strands comprise the cytosine analog that is resistant to TET-assisted pyridine borane treatment; subjecting the pluralities of the first and second strands to TET-assisted pyridine borane treatment under conditions such that any methylated cytosines if present in the first strands undergo cytosine conversion, wherein after cytosine conversion, the sequences of the first and second adaptor nucleic acids are no longer complementary; and detecting at least a portion of the plurality of first strands and at least a portion of the plurality of second strands.

In some aspects, the present disclosure provides methods of detecting cancer in an individual. In some embodiments, the methods are performed on a plurality of nucleic acids obtained from the individual (e.g., from a sample obtained from the individual), wherein the methylation level and/or somatic mutation(s) or lack thereof detected in the sample identify the individual as having or not having cancer. In some embodiments, the methods comprise: providing a plurality of first single-stranded DNA fragments; subjecting the plurality of first single-stranded DNA fragments to a round of primer extension in the presence of: (a) a primer that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands corresponding to the first single-stranded DNA fragments and a plurality of second strands that are complementary to the first strands, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion; and detecting at least a portion of the plurality of first strands and at least a portion of the plurality of second strands. In some embodiments, the methods comprise: providing a plurality of first single-stranded DNA fragments; subjecting the plurality of first single-stranded DNA fragments to a round of primer extension in the presence of: (a) a primer comprising: (i) a second adaptor nucleic acid portion that does not anneal to the first single-stranded DNA fragments and (ii) a portion that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands comprising the first single-stranded DNA fragments with a first adaptor nucleic acid complementary to the second adaptor nucleic acid and a plurality of second strands comprising second single-stranded DNA fragments complementary to the first single-stranded DNA fragments and the second adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and detecting at least a portion of the plurality of first strands and at least a portion of the plurality of second strands. In some embodiments, the methods comprise: providing a plurality of first single-stranded DNA fragments; attaching a first adaptor nucleic acid onto at least a portion of the first single-stranded DNA fragments of the plurality; subjecting the plurality of first single-stranded DNA fragments to a round of primer extension in the presence of: (a) a primer that anneals to at least a portion of the first single-stranded DNA fragments downstream or at the first adaptor nucleic acid. (b) a nucleic acid polymerase, and (c) a mixture of nucleotides comprising a cytosine analog that is resistant to cytosine conversion, thereby generating a plurality of first strands comprising the first single-stranded DNA fragments with the first adaptor nucleic acid and a plurality of second strands comprising second single-stranded DNA fragments complementary to the first single-stranded DNA fragments and a second adaptor nucleic acid complementary to the first adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and detecting at least a portion of the plurality of first strands and at least a portion of the plurality of second strands. In some embodiments, the methods comprise: providing a plurality of first single-stranded DNA fragments; subjecting the plurality of first single-stranded DNA fragments to a round of primer extension in the presence of: (a) a primer comprising: (i) a second adaptor nucleic acid portion comprising a non-complementary 5′ overhang that does not anneal to the first single-stranded DNA fragments, and (ii) a portion that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands comprising the first single-stranded DNA fragments with a first adaptor nucleic acid complementary to the portion of the second adaptor nucleic acid that anneals to the first single-stranded DNA fragments and a plurality of second strands comprising second single-stranded DNA fragments complementary to the first single-stranded DNA fragments and the second adaptor nucleic acid non-complementary to the first adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion; and detecting at least a portion of the plurality of first strands and at least a portion of the plurality of second strands.

In some aspects, the present disclosure provides methods of detecting minimal residual disease in an individual who has been treated or is undergoing treatment for cancer. In some embodiments, the methods are performed on a plurality of nucleic acids obtained from the individual (e.g., from a sample obtained from the individual), wherein the methylation level and/or somatic mutation(s) or lack thereof detected in the sample identify the individual as having minimal residual disease or a lack thereof. In some embodiments, the methods comprise: providing a plurality of first single-stranded DNA fragments; subjecting the plurality of first single-stranded DNA fragments to a round of primer extension in the presence of: (a) a primer that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands corresponding to the first single-stranded DNA fragments and a plurality of second strands that are complementary to the first strands, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion; and detecting at least a portion of the plurality of first strands and at least a portion of the plurality of second strands. In some embodiments, the methods comprise: providing a plurality of first single-stranded DNA fragments; subjecting the plurality of first single-stranded DNA fragments to a round of primer extension in the presence of: (a) a primer comprising: (i) a second adaptor nucleic acid portion that does not anneal to the first single-stranded DNA fragments and (ii) a portion that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands comprising the first single-stranded DNA fragments with a first adaptor nucleic acid complementary to the second adaptor nucleic acid and a plurality of second strands comprising second single-stranded DNA fragments complementary to the first single-stranded DNA fragments and the second adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and detecting at least a portion of the plurality of first strands and at least a portion of the plurality of second strands. In some embodiments, the methods comprise: providing a plurality of first single-stranded DNA fragments; attaching a first adaptor nucleic acid onto at least a portion of the first single-stranded DNA fragments of the plurality; subjecting the plurality of first single-stranded DNA fragments to a round of primer extension in the presence of: (a) a primer that anneals to at least a portion of the first single-stranded DNA fragments downstream or at the first adaptor nucleic acid, (b) a nucleic acid polymerase, and (c) a mixture of nucleotides comprising a cytosine analog that is resistant to cytosine conversion, thereby generating a plurality of first strands comprising the first single-stranded DNA fragments with the first adaptor nucleic acid and a plurality of second strands comprising second single-stranded DNA fragments complementary to the first single-stranded DNA fragments and a second adaptor nucleic acid complementary to the first adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and detecting at least a portion of the plurality of first strands and at least a portion of the plurality of second strands. In some embodiments, the methods comprise: providing a plurality of first single-stranded DNA fragments; subjecting the plurality of first single-stranded DNA fragments to a round of primer extension in the presence of: (a) a primer comprising: (i) a second adaptor nucleic acid portion comprising a non-complementary 5′ overhang that does not anneal to the first single-stranded DNA fragments, and (ii) a portion that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands comprising the first single-stranded DNA fragments with a first adaptor nucleic acid complementary to the portion of the second adaptor nucleic acid that anneals to the first single-stranded DNA fragments and a plurality of second strands comprising second single-stranded DNA fragments complementary to the first single-stranded DNA fragments and the second adaptor nucleic acid non-complementary to the first adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion; and detecting at least a portion of the plurality of first strands and at least a portion of the plurality of second strands.

In some aspects, the present disclosure provides methods of screening an individual suspected of having cancer. In some embodiments, the methods are performed on a plurality of nucleic acids obtained from the individual (e.g., from a sample obtained from the individual), wherein the methylation level and/or somatic mutation(s) or lack thereof detected in the sample identify the individual as likely or not likely to have cancer. In some embodiments, the methods comprise: providing a plurality of first single-stranded DNA fragments; subjecting the plurality of first single-stranded DNA fragments to a round of primer extension in the presence of: (a) a primer that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands corresponding to the first single-stranded DNA fragments and a plurality of second strands that are complementary to the first strands, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion; and detecting at least a portion of the plurality of first strands and at least a portion of the plurality of second strands. In some embodiments, the methods comprise: providing a plurality of first single-stranded DNA fragments; subjecting the plurality of first single-stranded DNA fragments to a round of primer extension in the presence of: (a) a primer comprising: (i) a second adaptor nucleic acid portion that does not anneal to the first single-stranded DNA fragments and (ii) a portion that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands comprising the first single-stranded DNA fragments with a first adaptor nucleic acid complementary to the second adaptor nucleic acid and a plurality of second strands comprising second single-stranded DNA fragments complementary to the first single-stranded DNA fragments and the second adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and detecting at least a portion of the plurality of first strands and at least a portion of the plurality of second strands. In some embodiments, the methods comprise: providing a plurality of first single-stranded DNA fragments; attaching a first adaptor nucleic acid onto at least a portion of the first single-stranded DNA fragments of the plurality; subjecting the plurality of first single-stranded DNA fragments to a round of primer extension in the presence of: (a) a primer that anneals to at least a portion of the first single-stranded DNA fragments downstream or at the first adaptor nucleic acid, (b) a nucleic acid polymerase, and (c) a mixture of nucleotides comprising a cytosine analog that is resistant to cytosine conversion, thereby generating a plurality of first strands comprising the first single-stranded DNA fragments with the first adaptor nucleic acid and a plurality of second strands comprising second single-stranded DNA fragments complementary to the first single-stranded DNA fragments and a second adaptor nucleic acid complementary to the first adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and detecting at least a portion of the plurality of first strands and at least a portion of the plurality of second strands. In some embodiments, the methods comprise: providing a plurality of first single-stranded DNA fragments; subjecting the plurality of first single-stranded DNA fragments to a round of primer extension in the presence of: (a) a primer comprising: (i) a second adaptor nucleic acid portion comprising a non-complementary 5′ overhang that does not anneal to the first single-stranded DNA fragments, and (ii) a portion that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands comprising the first single-stranded DNA fragments with a first adaptor nucleic acid complementary to the portion of the second adaptor nucleic acid that anneals to the first single-stranded DNA fragments and a plurality of second strands comprising second single-stranded DNA fragments complementary to the first single-stranded DNA fragments and the second adaptor nucleic acid non-complementary to the first adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion; and detecting at least a portion of the plurality of first strands and at least a portion of the plurality of second strands.

In some aspects, the present disclosure provides methods of determining prognosis of an individual having cancer. In some embodiments, the methods are performed on a plurality of nucleic acids obtained from the individual (e.g., from a sample obtained from the individual), wherein the methylation level and/or somatic mutation(s) or lack thereof detected in the sample determine at least in part the prognosis of the individual. In some embodiments, the methods comprise: providing a plurality of first single-stranded DNA fragments; subjecting the plurality of first single-stranded DNA fragments to a round of primer extension in the presence of: (a) a primer that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands corresponding to the first single-stranded DNA fragments and a plurality of second strands that are complementary to the first strands, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion; and detecting at least a portion of the plurality of first strands and at least a portion of the plurality of second strands. In some embodiments, the methods comprise: providing a plurality of first single-stranded DNA fragments; subjecting the plurality of first single-stranded DNA fragments to a round of primer extension in the presence of: (a) a primer comprising: (i) a second adaptor nucleic acid portion that does not anneal to the first single-stranded DNA fragments and (ii) a portion that anneals to at least a portion of the first single-stranded DNA fragments. (b) a nucleic acid polymerase, and (c) a mixture of nucleotides comprising a cytosine analog that is resistant to cytosine conversion, thereby generating a plurality of first strands comprising the first single-stranded DNA fragments with a first adaptor nucleic acid complementary to the second adaptor nucleic acid and a plurality of second strands comprising second single-stranded DNA fragments complementary to the first single-stranded DNA fragments and the second adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and detecting at least a portion of the plurality of first strands and at least a portion of the plurality of second strands. In some embodiments, the methods comprise: providing a plurality of first single-stranded DNA fragments; attaching a first adaptor nucleic acid onto at least a portion of the first single-stranded DNA fragments of the plurality; subjecting the plurality of first single-stranded DNA fragments to a round of primer extension in the presence of: (a) a primer that anneals to at least a portion of the first single-stranded DNA fragments downstream or at the first adaptor nucleic acid, (b) a nucleic acid polymerase, and (c) a mixture of nucleotides comprising a cytosine analog that is resistant to cytosine conversion, thereby generating a plurality of first strands comprising the first single-stranded DNA fragments with the first adaptor nucleic acid and a plurality of second strands comprising second single-stranded DNA fragments complementary to the first single-stranded DNA fragments and a second adaptor nucleic acid complementary to the first adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and detecting at least a portion of the plurality of first strands and at least a portion of the plurality of second strands. In some embodiments, the methods comprise: providing a plurality of first single-stranded DNA fragments; subjecting the plurality of first single-stranded DNA fragments to a round of primer extension in the presence of: (a) a primer comprising: (i) a second adaptor nucleic acid portion comprising a non-complementary 5′ overhang that does not anneal to the first single-stranded DNA fragments, and (ii) a portion that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands comprising the first single-stranded DNA fragments with a first adaptor nucleic acid complementary to the portion of the second adaptor nucleic acid that anneals to the first single-stranded DNA fragments and a plurality of second strands comprising second single-stranded DNA fragments complementary to the first single-stranded DNA fragments and the second adaptor nucleic acid non-complementary to the first adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion; and detecting at least a portion of the plurality of first strands and at least a portion of the plurality of second strands.

In some aspects, the present disclosure provides methods of predicting survival of an individual having cancer. In some embodiments, the methods are performed on a plurality of nucleic acids obtained from the individual (e.g., from a sample obtained from the individual), wherein the methylation level and/or somatic mutation(s) or lack thereof detected in the sample predict at least in part the survival of the individual. In some embodiments, the methods comprise: providing a plurality of first single-stranded DNA fragments; subjecting the plurality of first single-stranded DNA fragments to a round of primer extension in the presence of: (a) a primer that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands corresponding to the first single-stranded DNA fragments and a plurality of second strands that are complementary to the first strands, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion; and detecting at least a portion of the plurality of first strands and at least a portion of the plurality of second strands. In some embodiments, the methods comprise: providing a plurality of first single-stranded DNA fragments; subjecting the plurality of first single-stranded DNA fragments to a round of primer extension in the presence of: (a) a primer comprising: (i) a second adaptor nucleic acid portion that does not anneal to the first single-stranded DNA fragments and (ii) a portion that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands comprising the first single-stranded DNA fragments with a first adaptor nucleic acid complementary to the second adaptor nucleic acid and a plurality of second strands comprising second single-stranded DNA fragments complementary to the first single-stranded DNA fragments and the second adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and detecting at least a portion of the plurality of first strands and at least a portion of the plurality of second strands. In some embodiments, the methods comprise: providing a plurality of first single-stranded DNA fragments; attaching a first adaptor nucleic acid onto at least a portion of the first single-stranded DNA fragments of the plurality; subjecting the plurality of first single-stranded DNA fragments to a round of primer extension in the presence of: (a) a primer that anneals to at least a portion of the first single-stranded DNA fragments downstream or at the first adaptor nucleic acid, (b) a nucleic acid polymerase, and (c) a mixture of nucleotides comprising a cytosine analog that is resistant to cytosine conversion, thereby generating a plurality of first strands comprising the first single-stranded DNA fragments with the first adaptor nucleic acid and a plurality of second strands comprising second single-stranded DNA fragments complementary to the first single-stranded DNA fragments and a second adaptor nucleic acid complementary to the first adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and detecting at least a portion of the plurality of first strands and at least a portion of the plurality of second strands. In some embodiments, the methods comprise: providing a plurality of first single-stranded DNA fragments; subjecting the plurality of first single-stranded DNA fragments to a round of primer extension in the presence of: (a) a primer comprising: (i) a second adaptor nucleic acid portion comprising a non-complementary 5′ overhang that does not anneal to the first single-stranded DNA fragments, and (ii) a portion that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands comprising the first single-stranded DNA fragments with a first adaptor nucleic acid complementary to the portion of the second adaptor nucleic acid that anneals to the first single-stranded DNA fragments and a plurality of second strands comprising second single-stranded DNA fragments complementary to the first single-stranded DNA fragments and the second adaptor nucleic acid non-complementary to the first adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion; and detecting at least a portion of the plurality of first strands and at least a portion of the plurality of second strands.

In some aspects, the present disclosure provides methods of predicting or detecting tumor burden of an individual having cancer. In some embodiments, the methods are performed on a plurality of nucleic acids obtained from the individual (e.g., from a sample obtained from the individual), wherein the methylation level and/or somatic mutation(s) or lack thereof detected in the sample predict or detect at least in part the tumor burden of the individual. In some embodiments, the methods comprise: providing a plurality of first single-stranded DNA fragments; subjecting the plurality of first single-stranded DNA fragments to a round of primer extension in the presence of: (a) a primer that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands corresponding to the first single-stranded DNA fragments and a plurality of second strands that are complementary to the first strands, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion; and detecting at least a portion of the plurality of first strands and at least a portion of the plurality of second strands. In some embodiments, the methods comprise: providing a plurality of first single-stranded DNA fragments; subjecting the plurality of first single-stranded DNA fragments to a round of primer extension in the presence of: (a) a primer comprising: (i) a second adaptor nucleic acid portion that does not anneal to the first single-stranded DNA fragments and (ii) a portion that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands comprising the first single-stranded DNA fragments with a first adaptor nucleic acid complementary to the second adaptor nucleic acid and a plurality of second strands comprising second single-stranded DNA fragments complementary to the first single-stranded DNA fragments and the second adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and detecting at least a portion of the plurality of first strands and at least a portion of the plurality of second strands. In some embodiments, the methods comprise: providing a plurality of first single-stranded DNA fragments; attaching a first adaptor nucleic acid onto at least a portion of the first single-stranded DNA fragments of the plurality; subjecting the plurality of first single-stranded DNA fragments to a round of primer extension in the presence of: (a) a primer that anneals to at least a portion of the first single-stranded DNA fragments downstream or at the first adaptor nucleic acid, (b) a nucleic acid polymerase, and (c) a mixture of nucleotides comprising a cytosine analog that is resistant to cytosine conversion, thereby generating a plurality of first strands comprising the first single-stranded DNA fragments with the first adaptor nucleic acid and a plurality of second strands comprising second single-stranded DNA fragments complementary to the first single-stranded DNA fragments and a second adaptor nucleic acid complementary to the first adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and detecting at least a portion of the plurality of first strands and at least a portion of the plurality of second strands. In some embodiments, the methods comprise: providing a plurality of first single-stranded DNA fragments; subjecting the plurality of first single-stranded DNA fragments to a round of primer extension in the presence of: (a) a primer comprising: (i) a second adaptor nucleic acid portion comprising a non-complementary 5′ overhang that does not anneal to the first single-stranded DNA fragments, and (ii) a portion that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands comprising the first single-stranded DNA fragments with a first adaptor nucleic acid complementary to the portion of the second adaptor nucleic acid that anneals to the first single-stranded DNA fragments and a plurality of second strands comprising second single-stranded DNA fragments complementary to the first single-stranded DNA fragments and the second adaptor nucleic acid non-complementary to the first adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion; and detecting at least a portion of the plurality of first strands and at least a portion of the plurality of second strands.

In some aspects, the present disclosure provides methods of predicting responsiveness to treatment of an individual having cancer. In some embodiments, the methods are performed on a plurality of nucleic acids obtained from the individual (e.g., from a sample obtained from the individual), wherein the methylation level and/or somatic mutation(s) or lack thereof detected in the sample are used at least in part to predict responsiveness of the individual to a treatment. In some embodiments, the methods comprise: providing a plurality of first single-stranded DNA fragments; subjecting the plurality of first single-stranded DNA fragments to a round of primer extension in the presence of: (a) a primer that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands corresponding to the first single-stranded DNA fragments and a plurality of second strands that are complementary to the first strands, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion; and detecting at least a portion of the plurality of first strands and at least a portion of the plurality of second strands. In some embodiments, the methods comprise: providing a plurality of first single-stranded DNA fragments; subjecting the plurality of first single-stranded DNA fragments to a round of primer extension in the presence of: (a) a primer comprising: (i) a second adaptor nucleic acid portion that does not anneal to the first single-stranded DNA fragments and (ii) a portion that anneals to at least a portion of the first single-stranded DNA fragments. (b) a nucleic acid polymerase, and (c) a mixture of nucleotides comprising a cytosine analog that is resistant to cytosine conversion, thereby generating a plurality of first strands comprising the first single-stranded DNA fragments with a first adaptor nucleic acid complementary to the second adaptor nucleic acid and a plurality of second strands comprising second single-stranded DNA fragments complementary to the first single-stranded DNA fragments and the second adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and detecting at least a portion of the plurality of first strands and at least a portion of the plurality of second strands. In some embodiments, the methods comprise: providing a plurality of first single-stranded DNA fragments; attaching a first adaptor nucleic acid onto at least a portion of the first single-stranded DNA fragments of the plurality; subjecting the plurality of first single-stranded DNA fragments to a round of primer extension in the presence of: (a) a primer that anneals to at least a portion of the first single-stranded DNA fragments downstream or at the first adaptor nucleic acid, (b) a nucleic acid polymerase, and (c) a mixture of nucleotides comprising a cytosine analog that is resistant to cytosine conversion, thereby generating a plurality of first strands comprising the first single-stranded DNA fragments with the first adaptor nucleic acid and a plurality of second strands comprising second single-stranded DNA fragments complementary to the first single-stranded DNA fragments and a second adaptor nucleic acid complementary to the first adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and detecting at least a portion of the plurality of first strands and at least a portion of the plurality of second strands. In some embodiments, the methods comprise: providing a plurality of first single-stranded DNA fragments; subjecting the plurality of first single-stranded DNA fragments to a round of primer extension in the presence of: (a) a primer comprising: (i) a second adaptor nucleic acid portion comprising a non-complementary 5′ overhang that does not anneal to the first single-stranded DNA fragments, and (ii) a portion that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands comprising the first single-stranded DNA fragments with a first adaptor nucleic acid complementary to the portion of the second adaptor nucleic acid that anneals to the first single-stranded DNA fragments and a plurality of second strands comprising second single-stranded DNA fragments complementary to the first single-stranded DNA fragments and the second adaptor nucleic acid non-complementary to the first adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion; and detecting at least a portion of the plurality of first strands and at least a portion of the plurality of second strands.

In some aspects, the present disclosure provides methods of monitoring a cancer in an individual. In some embodiments, the methods are performed on a plurality of nucleic acids obtained from the individual (e.g., from a sample obtained from the individual). In some embodiments, the methods are performed on a first plurality of nucleic acids obtained from the individual (e.g., from a first sample obtained from the individual) and a second plurality of nucleic acids obtained from the individual (e.g., from a second sample obtained from the individual), wherein determining a difference in methylation level and/or somatic mutation(s) between the first and second samples is used to monitor the cancer in the individual. In some embodiments, the methods comprise: providing a plurality of first single-stranded DNA fragments; subjecting the plurality of first single-stranded DNA fragments to a round of primer extension in the presence of: (a) a primer that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands corresponding to the first single-stranded DNA fragments and a plurality of second strands that are complementary to the first strands, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion; and detecting at least a portion of the plurality of first strands and at least a portion of the plurality of second strands. In some embodiments, the methods comprise: providing a plurality of first single-stranded DNA fragments; subjecting the plurality of first single-stranded DNA fragments to a round of primer extension in the presence of: (a) a primer comprising: (i) a second adaptor nucleic acid portion that does not anneal to the first single-stranded DNA fragments and (ii) a portion that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands comprising the first single-stranded DNA fragments with a first adaptor nucleic acid complementary to the second adaptor nucleic acid and a plurality of second strands comprising second single-stranded DNA fragments complementary to the first single-stranded DNA fragments and the second adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and detecting at least a portion of the plurality of first strands and at least a portion of the plurality of second strands. In some embodiments, the methods comprise: providing a plurality of first single-stranded DNA fragments; attaching a first adaptor nucleic acid onto at least a portion of the first single-stranded DNA fragments of the plurality; subjecting the plurality of first single-stranded DNA fragments to a round of primer extension in the presence of: (a) a primer that anneals to at least a portion of the first single-stranded DNA fragments downstream or at the first adaptor nucleic acid. (b) a nucleic acid polymerase, and (c) a mixture of nucleotides comprising a cytosine analog that is resistant to cytosine conversion, thereby generating a plurality of first strands comprising the first single-stranded DNA fragments with the first adaptor nucleic acid and a plurality of second strands comprising second single-stranded DNA fragments complementary to the first single-stranded DNA fragments and a second adaptor nucleic acid complementary to the first adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and detecting at least a portion of the plurality of first strands and at least a portion of the plurality of second strands. In some embodiments, the methods comprise: providing a plurality of first single-stranded DNA fragments; subjecting the plurality of first single-stranded DNA fragments to a round of primer extension in the presence of: (a) a primer comprising: (i) a second adaptor nucleic acid portion comprising a non-complementary 5′ overhang that does not anneal to the first single-stranded DNA fragments, and (ii) a portion that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands comprising the first single-stranded DNA fragments with a first adaptor nucleic acid complementary to the portion of the second adaptor nucleic acid that anneals to the first single-stranded DNA fragments and a plurality of second strands comprising second single-stranded DNA fragments complementary to the first single-stranded DNA fragments and the second adaptor nucleic acid non-complementary to the first adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion; and detecting at least a portion of the plurality of first strands and at least a portion of the plurality of second strands.

In some aspects, the present disclosure provides methods of monitoring response of an individual being treated for cancer. In some embodiments, the methods are performed on a plurality of nucleic acids obtained from the individual (e.g., from a sample obtained from the individual), e.g., after treatment. In some embodiments, the methods comprise administering a treatment to the individual and detecting methylation level and/or somatic mutation(s) or lack thereof, wherein the methylation level and/or somatic mutation(s) or lack thereof detected in the sample are used at least in part to monitor response to the treatment. In some embodiments, the methods are performed on a first plurality of nucleic acids obtained from the individual (e.g., from a first sample obtained from the individual) and a second plurality of nucleic acids obtained from the individual (e.g., from a second sample obtained from the individual), wherein determining a difference in methylation level and/or somatic mutation(s) between the first and second samples is used to monitor the cancer in the individual. In some embodiments, the methods comprise: providing a plurality of first single-stranded DNA fragments; subjecting the plurality of first single-stranded DNA fragments to a round of primer extension in the presence of: (a) a primer that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands corresponding to the first single-stranded DNA fragments and a plurality of second strands that are complementary to the first strands, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion; and detecting at least a portion of the plurality of first strands and at least a portion of the plurality of second strands. In some embodiments, the methods comprise: providing a plurality of first single-stranded DNA fragments; subjecting the plurality of first single-stranded DNA fragments to a round of primer extension in the presence of: (a) a primer comprising: (i) a second adaptor nucleic acid portion that does not anneal to the first single-stranded DNA fragments and (ii) a portion that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands comprising the first single-stranded DNA fragments with a first adaptor nucleic acid complementary to the second adaptor nucleic acid and a plurality of second strands comprising second single-stranded DNA fragments complementary to the first single-stranded DNA fragments and the second adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and detecting at least a portion of the plurality of first strands and at least a portion of the plurality of second strands. In some embodiments, the methods comprise: providing a plurality of first single-stranded DNA fragments; attaching a first adaptor nucleic acid onto at least a portion of the first single-stranded DNA fragments of the plurality; subjecting the plurality of first single-stranded DNA fragments to a round of primer extension in the presence of: (a) a primer that anneals to at least a portion of the first single-stranded DNA fragments downstream or at the first adaptor nucleic acid, (b) a nucleic acid polymerase, and (c) a mixture of nucleotides comprising a cytosine analog that is resistant to cytosine conversion, thereby generating a plurality of first strands comprising the first single-stranded DNA fragments with the first adaptor nucleic acid and a plurality of second strands comprising second single-stranded DNA fragments complementary to the first single-stranded DNA fragments and a second adaptor nucleic acid complementary to the first adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and detecting at least a portion of the plurality of first strands and at least a portion of the plurality of second strands. In some embodiments, the methods comprise: providing a plurality of first single-stranded DNA fragments; subjecting the plurality of first single-stranded DNA fragments to a round of primer extension in the presence of: (a) a primer comprising: (i) a second adaptor nucleic acid portion comprising a non-complementary 5′ overhang that does not anneal to the first single-stranded DNA fragments, and (ii) a portion that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands comprising the first single-stranded DNA fragments with a first adaptor nucleic acid complementary to the portion of the second adaptor nucleic acid that anneals to the first single-stranded DNA fragments and a plurality of second strands comprising second single-stranded DNA fragments complementary to the first single-stranded DNA fragments and the second adaptor nucleic acid non-complementary to the first adaptor nucleic acid, wherein the second strands comprise the cytosine analog that is resistant to cytosine conversion; subjecting the pluralities of the first and second strands to a cytosine conversion treatment under conditions such that unmethylated cytosine(s) if present in the first strands undergo cytosine conversion; and detecting at least a portion of the plurality of first strands and at least a portion of the plurality of second strands.

### Detection

In some embodiments, detecting (e.g., at least a portion of the plurality of first strands and/or at least a portion of the plurality of second strands of the present disclosure) is by microarray, quantitative PCR (qPCR), digital droplet PCR (ddPCR), molecular inversion probes, or sequencing.

In some embodiments, at least a portion of the plurality of first strands and at least a portion of the plurality of second strands of the present disclosure are detected by sequencing. In some embodiments, a plurality of sequence reads is obtained from at least a portion of a plurality of first strands of the present disclosure. In some embodiments, a plurality of sequence reads is obtained from at least a portion of a plurality of second strands of the present disclosure. In some embodiments, the sequencing is whole-genome methyl sequencing (WGMS) or next-generation sequencing (NGS). For example, in some embodiments, at least a portion of a plurality of first strands of the present disclosure (e.g., the methylation strand) is detected via WGMS (e.g., enzymatic methylation sequencing) and/or at least a portion of a plurality of second strands of the present disclosure and/or their amplification products (e.g., the genomic strand) is detected via WGS (e.g., NGS).

Various methods for WGMS are known in the art. Generally, these methods combine cytosine conversion (e.g., using the methods described supra) with whole-genome sequencing techniques. For example, in some embodiments, the WGMS comprises bisulfite sequencing, whole genome bisulfite sequencing (WGBS), APOBEC-seq, methyl-CpG-binding domain (MBD) protein capture, methyl-DNA immunoprecipitation (MeDIP-seq), methylation sensitive restriction enzyme sequencing (MSRE/MRE-Seq or Methyl-Seq), enzymatic methylation sequencing, oxidative bisulfite sequencing (oxBS-Seq), reduced representative bisulfite sequencing (RRBS), or Tet-assisted bisulfite sequencing (TAB-Seq).

Some WGMS methods rely upon library construction and adapter ligation, followed by standard bisulfite conversion and sequencing (e.g., WGBS). Alternatively, bisulfite treatment can be carried out prior to adaptor ligation (see, e.g., Miura, F. et al. (2012) Nucleic Acids Res. 40: e136). More recent techniques use other cytosine conversion methods such as enzymatic approaches in order to reduce damage to DNA caused by bisulfite, e.g., as in the commercially available NEBNext® Enzymatic Methyl-seq Kit (New England Biolabs). Steps of library amplification, quantification, and sequencing generally follow bisulfite conversion. In some embodiments, prior to WGMS, nucleic acids are extracted from a sample. In some embodiments, prior to WGMS, nucleic acids are subjected to fragmentation, repair, and adaptor ligation. As noted previously, cytosine conversion can be carried out before or after adaptor ligation. In some embodiments, DNA repair is performed after cytosine conversion. PCR amplification (generally at least two cycles) is performed after cytosine conversion to convert uracils (generated by formerly unmethylated cytosines) into thymine, and is accomplished using a polymerase that is able to read uracil (excluding polymerases with proofreading and repair activities). In some embodiments, prior to sequencing, fragments are enriched for desired length. In some embodiments, prior to sequencing, nucleic acids are enriched for methylated sequences, such as by immunoprecipitation using an antibody specific for 5mC as in the MeDIP approach (see, e.g., Pomraning, K. R. et al. (2009) Methods 47:142-150.

NGS methods are known in the art, and are described, e.g., in Metzker, M. (2010) Nature Biotechnology Reviews 11:31-46. Platforms for next-generation sequencing include, e.g., Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLID) system, Polonator's G.007 system. Helicos BioSciences' HeliScope Gene Sequencing system, and Pacific Biosciences' PacBio RS system. NGS technologies can include one or more of steps, e.g., template preparation, sequencing and imaging, and data analysis. Methods for template preparation can include steps such as randomly breaking nucleic acids (e.g., genomic DNA) into smaller sizes and generating sequencing templates (e.g., fragment templates or mate-pair templates). The spatially separated templates can be attached or immobilized to a solid surface or support, allowing massive amounts of sequencing reactions to be performed simultaneously. Types of templates that can be used for NGS reactions include, e.g., clonally amplified templates originating from single DNA molecules, and single DNA molecule templates. Exemplary sequencing and imaging steps for NGS include, e.g., cyclic reversible termination (CRT), sequencing by ligation (SBL), single-molecule addition (pyrosequencing), and real-time sequencing. After NGS reads have been generated, they can be aligned to a known reference sequence or assembled de novo. For example, identifying genetic variations such as single-nucleotide polymorphism and structural variants in a sample (e.g., a tumor sample) can be accomplished by aligning NGS reads to a reference sequence (e.g., a wild type sequence). Methods of sequence alignment for NGS are described e.g., in Trapnell C. and Salzberg S. L. Nature Biotech., 2009, 27:455-457. Examples of de novo assemblies are described, e.g., in Warren R. et al., Bioinformatics, 2007, 23:500-501; Butler J. et al., Genome Res., 2008, 18:810-820; and Zerbino D. R. and Birney E., Genome Res., 2008, 18:821-829. Sequence alignment or assembly can be performed using read data from one or more NGS platforms, e.g., mixing Roche/454 and Illumina/Solexa read data. In some embodiments, NGS is performed according to the methods described in, e.g., Frampton, G. M. et al. (2013) Nat. Biotech. 31:1023-1031; and/or Montesion, M., et al., Cancer Discovery (2021) 11 (2): 282-92.

In some embodiments, the methods further comprise, prior to sequencing the plurality of polynucleotides or providing a plurality of sequence reads: subjecting a plurality of nucleic acids to fragmentation. A variety of DNA fragmentation techniques are used in the art prior to NGS or WGMS approaches. In some embodiments, nucleic acids are fragmented by nebulization, in which compressed gas is used to mechanically shear nucleic acids through a small opening. In some embodiments, nucleic acids are fragmented by sonication, in which ultrasonic waves are used to shear nucleic acids. In some embodiments, nucleic acids are fragmented enzymatically, e.g., using one or more enzymes to digest nucleic acids into fragments. See, e.g., the NEBNext® dsDNA Fragmentase, a mixture of two enzymes: one that randomly generates dsDNA nicks, and one that recognizes nicked sites and cuts the opposite strand, generating dsDNA breaks.

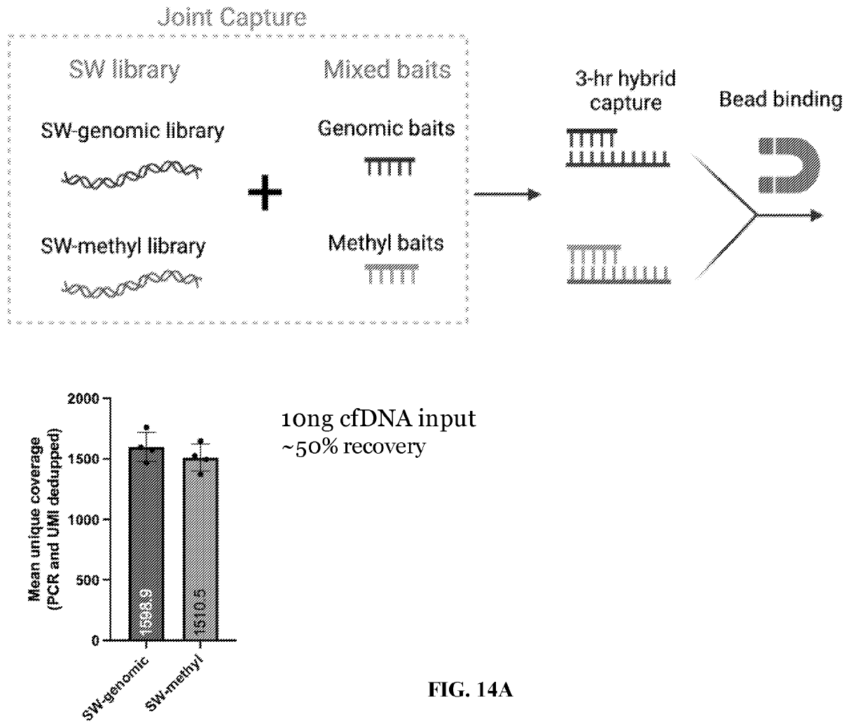

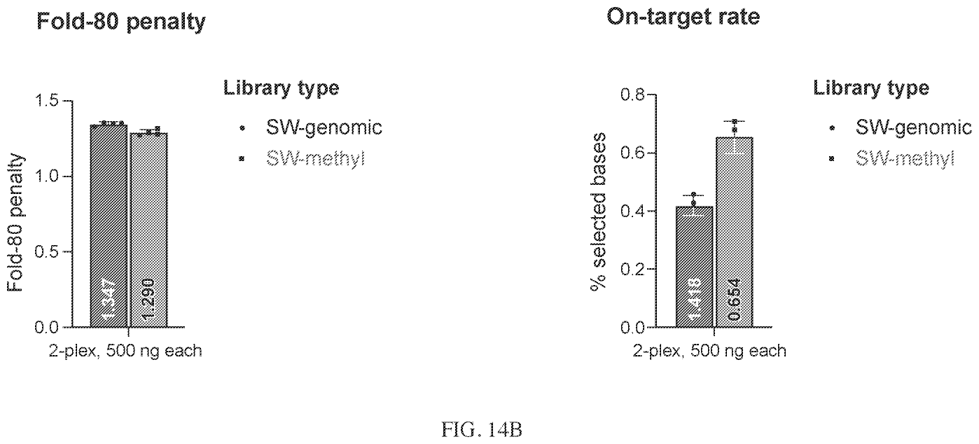

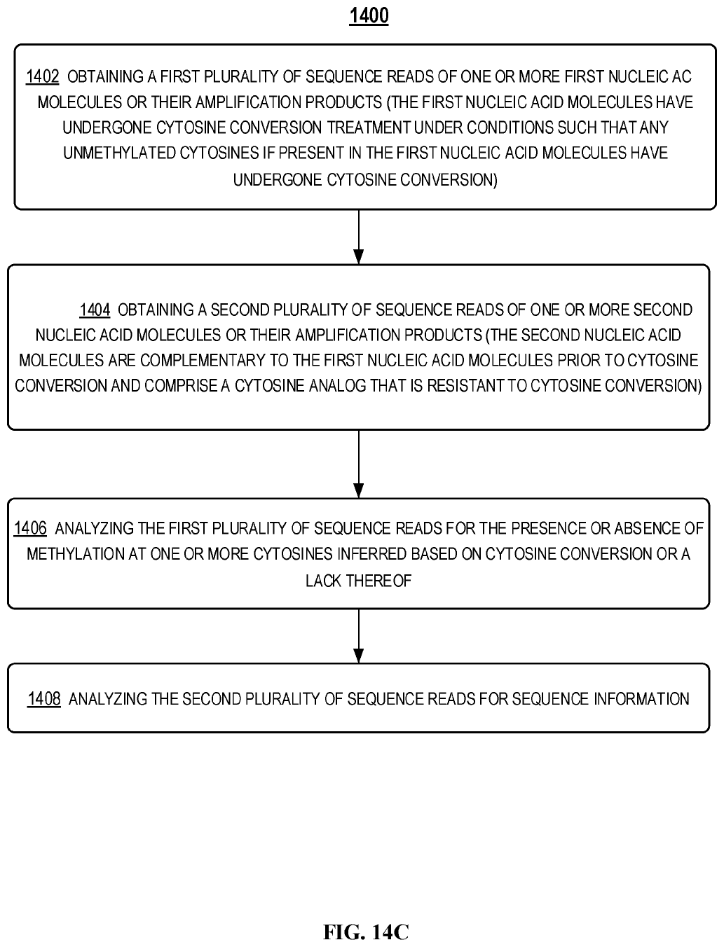

In some embodiments, the methods further comprise, prior to sequencing the plurality of polynucleotides or providing a plurality of sequence reads: selectively enriching for a plurality of nucleic acids or nucleic acid fragments, e.g., the methylation and/or genomic strand(s), as described above. For example, one or more baits or probes can be used to hybridize with a genomic locus of interest or fragment thereof, e.g., comprising a cluster of two or more CpG dinucleotides or comprising a genetic variant/mutation of interest. See, e.g., Graham, B. I. et al. Twist Fast Hybridization targeted methylation sequencing: a tunable target enrichment solution for methylation detection [abstract]. In: Proceedings of the American Association for Cancer Research Annual Meeting 2021; 2021 Apr. 10-15 and May 17-21. Philadelphia (PA): AACR; Cancer Res 2021; 81 (13_Suppl): Abstract nr 2098. In some embodiments, two or more baits or probes are used: one set of bait(s) or probe(s) for selectively enriching for the methylation strand(s), and one set of bait(s) or probe(s) for selectively enriching for the genomic strand(s). In some embodiments, two or more baits or probes are used: one set of bait(s) or probe(s) for selectively enriching for a library generated using the methylation strand(s), and one set of bait(s) or probe(s) for selectively enriching for a library generated using the genomic strand(s). Thus, joint hybrid capture for both methylation and genomic data can be achieved, resulting in deep coverage and information for methylation and genomic information in a single workflow, e.g., as illustrated in FIG. 14B.

In some embodiments, the methods further comprise, prior to sequencing the plurality of polynucleotides or providing a plurality of sequence reads: amplifying a plurality of nucleic acids or nucleic acid fragments by polymerase chain reaction (PCR). A variety of PCR techniques suitable for WGMS and NGS are known in the art. As noted above, in some embodiments, a plurality of nucleic acids or nucleic acid fragments is amplified by PCR after cytosine conversion. In some embodiments, the PCR is accomplished using a cytosine analog of the present disclosure.

In some embodiments, the methods further comprise, prior to sequencing the plurality of polynucleotides or providing a plurality of sequence reads: contacting a mixture of polynucleotides with the bait molecule under conditions suitable for hybridization, wherein the mixture comprises a plurality of polynucleotides capable of hybridization with the bait molecule; and isolating a plurality of polynucleotides that hybridized with the bait molecule, wherein the isolated plurality of polynucleotides that hybridized with the bait molecule are sequenced by NGS.

In some embodiments, a plurality of sequence reads is obtained by performing sequencing on nucleic acids captured by hybridization with a bait molecule. In some embodiments, the plurality of sequence reads was obtained by performing whole exome sequencing on nucleic acids captured by hybridization with a bait molecule. In some embodiments, the plurality of sequence reads was obtained by performing next-generation sequencing (NGS), whole exome sequencing, or methylation sequencing (e.g., WGMS) on nucleic acids captured by hybridization with the bait molecule.

In some embodiments, a hybrid capture approach is used. Further details about this and other hybrid capture processes can be found in U.S. Pat. No. 9,340,830; Frampton, G. M. et al. (2013) Nat. Biotech. 31:1023-1031; and Montesion, M., et al., Cancer Discovery (2021) 11 (2): 282-92. In some embodiments, the methods further comprise, prior to contacting the mixture of polynucleotides with the bait molecule: obtaining a sample from an individual, wherein the sample comprises tumor cells and/or tumor nucleic acids; and extracting the mixture of polynucleotides from the sample, wherein the mixture of polynucleotides is from the tumor cells and/or tumor nucleic acids. In some embodiments, the sample further comprises non-tumor cells.

In some embodiments, a plurality of sequence reads of the present disclosure includes paired-end sequence reads. Generally, paired-end sequencing methodologies are described, e.g., in WO2007/010252, WO2007/091077, and WO03/74734. This approach utilizes pairwise sequencing of a double-stranded polynucleotide template, which results in the sequential determination of nucleotide sequences in two distinct and separate regions of the polynucleotide template. The paired-end methodology makes it possible to obtain two linked or paired reads of sequence information from each double-stranded template on a clustered array, rather than just a single sequencing read as can be obtained with other methods. Paired end sequencing technology can make special use of clustered arrays, generally formed by solid-phase amplification, for example as set forth in WO03/74734. Target polynucleotide duplexes, fitted with adapters, are immobilized to a solid support at the 5′ ends of each strand of each duplex, for example, via bridge amplification as described above, forming dense clusters of double stranded DNA. Because both strands are immobilized at their 5′ ends, sequencing primers are then hybridized to the free 3′ end and sequencing by synthesis is performed. Adapter sequences can be inserted in between target sequences to allow for up to four reads from each duplex, as described in WO2007/091077. In a further adaptation of this methodology, specific strands can be cleaved in a controlled fashion as set forth in WO2007/010252. As a result, the timing of the sequencing read for each strand can be controlled, permitting sequential determination of the nucleotide sequences in two distinct and separate regions on complementary strands of the double-stranded template. See, e.g., U.S. Pat. No. 10,174,372.

In some embodiments, the plurality of sequence reads includes unpaired sequence reads.

In some embodiments, the methods of the present disclosure comprise demultiplexing sequence information from the first and/or second strands. In some embodiments, the methods of the present disclosure comprise demultiplexing sequence information in order to distinguish sequence reads from the first or methylation strands vs. sequence reads from the second or genomic strands. In some embodiments, the demultiplexing is based at least in part on first and/or second adaptor nucleic acids of the present disclosure. Other capabilities include the potential to use an NGS index sequence to identify not only the sample, but the strand modality (methyl or genetic)

In some embodiments, the methods further comprise comparing a sequence of the plurality of the second strands with a sequence of the plurality of the first strands. For example, a sequence from the methylation strand can be compared to a corresponding sequence from the genomic strand, e.g., in order to detect conversion or lack thereof at one or more cytosines.

In some embodiments, the methods further comprise comparing a sequence from the plurality of the first and/or the second strands with a reference genome sequence. For example, a sequence from the methylation strand can be compared to a corresponding sequence from the reference genome, e.g., in order to detect conversion or lack thereof at one or more cytosines, or a sequence from the genomic strand can be compared to a corresponding sequence from the reference genome, e.g., in order to detect a sequence variant/mutation.

In some embodiments, the methods of the present disclosure further comprise, prior to determining a consensus methylation pattern and CCF: performing alignment of sequence reads from the plurality to a reference genome, e.g., a human reference genome. In some embodiments, the alignment is a three-letter alignment to a human reference genome. In some embodiments, the methods of the present disclosure further comprise, prior to determining a consensus methylation pattern and CCF: excluding sequencing reads from the plurality that failed to undergo cytosine conversion. In some embodiments, the methods of the present disclosure further comprise, prior to determining a consensus methylation pattern and CCF: excluding sequence reads with a base other than cytosine or thymine at a first position of at least one of the CpG dinucleotides. For example, these can be due to sequencing errors or mutations (somatic or germline). In some embodiments, the methods of the present disclosure further comprise, prior to determining a consensus methylation pattern and CCF: excluding sequence reads with a base quality below a threshold base quality. In some embodiments, base calls at a cytosine within a CpG dinucleotide are determined using two overlapping paired-end sequence reads.

In some embodiments, detecting (e.g., at least a portion of the plurality of first strands and/or at least a portion of the plurality of second strands of the present disclosure) is by microarray. Microarray techniques suitable for detection of genetic variants (e.g., based on the genomic strand) are known in the art; in some embodiments, the microarray comprises probe(s) specific for one or more genetic variants/mutations of interest. Microarray techniques suitable for detection of methylation (e.g., based on the methylation strand) are known in the art; see, e.g., Deatherage, D. E. et al. (2009) Methods Mol. Biol. 556:117-139.

In some embodiments, detecting (e.g., at least a portion of the plurality of first strands and/or at least a portion of the plurality of second strands of the present disclosure) is by quantitative PCR (qPCR). qPCR techniques suitable for detection of genetic variants (e.g., based on the genomic strand) are known in the art; in some embodiments, the qPCR uses primers and/or probe(s) specific for one or more genetic variants/mutations of interest. qPCR techniques suitable for detection of methylation (e.g., based on the methylation strand) are known in the art; in some embodiments, the qPCR uses primers and/or probe(s) specific for methylation status at particular methylated/unmethylated cytosine(s) (see, e.g., Dugast-Darzacq. C. and Grange, T. (2009) Methods Mol. Biol. 507:281-303.

In some embodiments, detecting (e.g., at least a portion of the plurality of first strands and/or at least a portion of the plurality of second strands of the present disclosure) is by digital droplet PCR (ddPCR). ddPCR techniques suitable for detection of genetic variants (e.g., based on the genomic strand) are known in the art; in some embodiments, the ddPCR uses primers and/or probe(s) specific for one or more genetic variants/mutations of interest. ddPCR techniques suitable for detection of methylation (e.g., based on the methylation strand) are known in the art; see, e.g., Yu, M. et al. (2018) Methods Mol. Biol. 1768:363-383.

In some embodiments, detecting (e.g., at least a portion of the plurality of first strands and/or at least a portion of the plurality of second strands of the present disclosure) is molecular inversion probe(s) (MIPs). MIP techniques suitable for detection of genetic variants (e.g., based on the genomic strand) are known in the art; see, e.g., Absalan, F. and Ronaghi, M. (2007) Methods Mol Biol. 396:315-330. MIP techniques suitable for detection of methylation (e.g., based on the methylation strand) are also known in the art; see, e.g., Carrascosa, L. G. et al. (2014) Chem Commun (Camb) 50:3585-3588.

### Samples and Cancers

In some embodiments, single- and/or double-stranded DNA fragments of the present disclosure are obtained from a sample.

In some embodiments, the methods of the present disclosure further comprise isolating a plurality of nucleic acids from a sample. In some embodiments, nucleic acids are obtained from a sample, e.g., comprising tumor cells and/or tumor nucleic acids. For example, the sample can comprise tumor cell(s), circulating tumor cell(s), tumor nucleic acids (e.g., tumor circulating tumor DNA, cfDNA, or cfRNA), part or all of a tumor biopsy, fluid, cells, tissue, mRNA, cDNA, DNA, RNA, cell-free DNA, and/or cell-free RNA. In some embodiments, the sample is from a tumor biopsy or tumor specimen. In some embodiments, the sample further comprises non-tumor cells and/or non-tumor nucleic acids. In some embodiments, the fluid comprises blood, serum, plasma, saliva, semen, cerebral spinal fluid, amniotic fluid, peritoneal fluid, interstitial fluid, etc. In some embodiments, the sample further comprises non-tumor cells and/or non-tumor nucleic acids.

In some embodiments, a sample comprises tissue, cells, and/or nucleic acids from a cancer and/or tissue, cells, and/or nucleic acids from normal tissue. In some embodiments, the sample comprises a tissue biopsy sample, a liquid biopsy sample, or a normal control. In some embodiments, the sample is from a tumor biopsy, tumor specimen, or circulating tumor cell. In some embodiments, the sample is a liquid biopsy sample and comprises blood, plasma, serum, cerebrospinal fluid, sputum, stool, urine, or saliva.

In some embodiments, the sample comprises a fraction of tumor nucleic acids that is less than 1% of total nucleic acids, less than 0.5% of total nucleic acids, less than 0.1% of total nucleic acids, or less than 0.05% of total nucleic acids. In some embodiments, the sample comprises a fraction of tumor nucleic acids that is at least 0.01%, at least 0.05%, or at least 0.1% of total nucleic acids. In some embodiments, the sample comprises a fraction of tumor nucleic acids having an upper limit of 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, or 0.02% of total nucleic acids and an independently selected lower limit of 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%. 0.006%, 0.007%, 0.008%, 0.009%, 0.01%, 0.02%, 0.03%, 0.04%, 0.05%, 0.06%, 0.07%, 0.08%. 0.09%, 0.1%, 0.2%, 0.3%, 0.4%, 0.5%, 0.6%, 0.7%, 0.8%, 0.9%, or 1% of total nucleic acids, wherein the upper limit is greater than the lower limit.

In some embodiments, the sample is or comprises biological tissue or fluid. The sample can contain compounds that are not naturally intermixed with the tissue in nature such as preservatives, anticoagulants, buffers, fixatives, nutrients, antibiotics or the like. In one embodiment, the sample is preserved as a frozen sample or as a formaldehyde- or paraformaldehyde-fixed paraffin-embedded (FFPE) tissue preparation. For example, the sample can be embedded in a matrix, e.g., an FFPE block or a frozen sample. In another embodiment, the sample is a blood or blood constituent sample. In yet another embodiment, the sample is a bone marrow aspirate sample. In another embodiment, the sample comprises cell-free DNA (cfDNA) or circulating cell-free DNA (ccfDNA), e.g., tumor cfDNA or tumor ccfDNA. Without wishing to be bound by theory, it is believed that in some embodiments, cfDNA is DNA from apoptosed or necrotic cells. Typically, cfDNA is bound by protein (e.g., histone) and protected by nucleases. CfDNA can be used as a biomarker, for example, for non-invasive prenatal testing (NIPT), organ transplant, cardiomyopathy, microbiome, and cancer. In another embodiment, the sample comprises circulating tumor DNA (ctDNA). Without wishing to be bound by theory, it is believed that in some embodiments, ctDNA is cfDNA with a genetic or epigenetic alteration (e.g., a somatic alteration or a methylation signature) that can discriminate it originating from a tumor cell versus a non-tumor cell. In another embodiment, the sample comprises circulating tumor cells (CTCs). Without wishing to be bound by theory, it is believed that in some embodiments, CTCs are cells shed from a primary or metastatic tumor into the circulation. In some embodiments, CTCs apoptose and are a source of ctDNA in the blood/lymph.

In some embodiments of any of the methods provided herein, the cancer is a carcinoma, a sarcoma, a lymphoma, a leukemia, a myeloma, a germ cell cancer, or a blastoma. In some embodiments, the cancer is a solid tumor. In some embodiments, the cancer is a hematologic malignancy. In some embodiments, the cancer is a B cell cancer, a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma. Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypercosinophilia, chronic eosinophilic leukemia, neuroendocrine cancers, or a carcinoid tumor.

In some embodiments, the cancer is appendix adenocarcinoma, bladder adenocarcinoma, bladder urothelial (transitional cell) carcinoma, breast cancer not otherwise specified (NOS), breast carcinoma NOS, breast invasive ductal carcinoma (IDC), breast invasive lobular carcinoma (ILC), cervix squamous cell carcinoma (SCC), colon adenocarcinoma (CRC), esophagus adenocarcinoma, esophagus carcinoma NOS, esophagus squamous cell carcinoma (SCC), eye intraocular melanoma, gallbladder adenocarcinoma, gastroesophageal junction adenocarcinoma, intra-hepatic cholangiocarcinoma, kidney cancer NOS, liver hepatocellular carcinoma (HCC), lung cancer NOS, lung adenocarcinoma, lung large cell carcinoma, lung non-small cell lung carcinoma (NSCLC) NOS, lung small cell undifferentiated carcinoma, lung squamous cell carcinoma (SCC), ovary cancer NOS, pancreas cancer NOS, pancreas ductal adenocarcinoma, pancreatobiliary carcinoma, prostate cancer NOS, prostate acinar adenocarcinoma, prostate ductal adenocarcinoma, rectum adenocarcinoma (CRC), skin melanoma, small intestine adenocarcinoma, soft tissue sarcoma NOS, stomach adenocarcinoma NOS, unknown primary cancer NOS, unknown primary adenocarcinoma, unknown primary carcinoma (CUP) NOS, unknown primary neuroendocrine tumor, unknown primary squamous cell carcinoma (SCC), or uterus endometrial adenocarcinoma NOS.

In some embodiments, a sample of the present disclosure is obtained from an individual. In some embodiments, the individual has cancer. In some embodiments, the individual is suspected of having cancer. In some embodiments, the individual is being screened for cancer, or a recurrence or remission thereof. In some embodiments, the individual is undergoing or has undergone a treatment, e.g., for cancer.

### Software, Systems, and Devices

In another aspect, provided herein are systems comprising one or more processors; and a memory configured to store one or more computer program instructions, wherein the one or more computer program instructions when executed by the one or more processors are configured to: obtain a first plurality of sequence reads of one or more first nucleic acid molecules or their amplification products, wherein the first nucleic acid molecules have undergone cytosine conversion treatment under conditions such that unmethylated cytosine(s) if present in the first nucleic acid molecules have undergone cytosine conversion; obtain a second plurality of sequence reads of one or more second nucleic acid molecules or their amplification products, wherein the second nucleic acid molecules are complementary to the first nucleic acid molecules prior to cytosine conversion and comprise a cytosine analog that is resistant to cytosine conversion; analyze the first plurality of sequence reads for the presence or absence of methylation at one or more cytosines inferred based on cytosine conversion or a lack thereof; and analyze the second plurality of sequence reads for sequence information. In some embodiments, the one or more first nucleic acid molecules or their amplification products further comprise a first adaptor nucleic acid sequence, and wherein the one or more computer program instructions when executed by the one or more processors are further configured to: demultiplex the first and second pluralities of sequence reads based at least in part on detection of the first adaptor nucleic acid sequence in the first plurality of sequence reads. In some embodiments, the one or more second nucleic acid molecules or their amplification products further comprise a second adaptor nucleic acid sequence, and wherein the one or more computer program instructions when executed by the one or more processors are further configured to: demultiplex the first and second pluralities of sequence reads based at least in part on detection of the second adaptor nucleic acid sequence in the second plurality of sequence reads. In some embodiments, the first plurality of sequence reads is at least 2×, at least 4×, at least 8×, at least 10×, at least 50×, at least 100×, or at least 1000× more abundant than the second plurality of sequence reads. In some embodiments, the second plurality of sequence reads is at least 2×, at least 4×, at least 8×, at least 10×, at least 50×, at least 100×, or at least 1000× more abundant than the first plurality of sequence reads. In some embodiments, the first and/or second plurality of sequence reads is obtained by sequencing; optionally wherein the sequencing comprises use of a massively parallel sequencing (MPS) technique, whole genome sequencing (WGS), whole exome sequencing, targeted sequencing, direct sequencing, or a Sanger sequencing technique; and optionally wherein the massively parallel sequencing technique comprises next generation sequencing (NGS). In some embodiments, the one or more program instructions when executed by the one or more processors are further configured to generate, based at least in part on the analyzing, a molecular profile for the sample. In some embodiments, the molecular profile further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the individual is administered a treatment based at least in part on the molecular profile. In some embodiments, the molecular profile further comprises results from a nucleic acid sequencing-based test. In some embodiments, the one or more computer program instructions when executed by the one or more processors are further configured to: compare a sequence read of the second plurality of sequence reads with a sequence read of the first plurality of sequence reads. In some embodiments, the one or more computer program instructions when executed by the one or more processors are further configured to: compare one or more sequence read(s) of the first and/or second plurality of sequence reads with a reference genome sequence.

In another aspect, provided herein are non-transitory computer readable storage media comprising one or more programs executable by one or more computer processors for performing a method, comprising: obtaining a first plurality of sequence reads of one or more first nucleic acid molecules or their amplification products, wherein the first nucleic acid molecules have undergone cytosine conversion treatment under conditions such that unmethylated cytosine(s) if present in the first nucleic acid molecules have undergone cytosine conversion; obtaining a second plurality of sequence reads of one or more second nucleic acid molecules or their amplification products, wherein the second nucleic acid molecules are complementary to the first nucleic acid molecules prior to cytosine conversion and comprise a cytosine analog that is resistant to cytosine conversion; analyzing the first plurality of sequence reads for the presence or absence of methylation at one or more cytosines inferred based on cytosine conversion or a lack thereof; and analyzing the second plurality of sequence reads for sequence information. In some embodiments, the one or more first nucleic acid molecules or their amplification products further comprise a first adaptor nucleic acid sequence, and the method further comprises: demultiplexing the first and second pluralities of sequence reads based at least in part on detection of the first adaptor nucleic acid sequence in the first plurality of sequence reads. In some embodiments, the one or more second nucleic acid molecules or their amplification products further comprise a second adaptor nucleic acid sequence, and the method further comprises: demultiplexing the first and second pluralities of sequence reads based at least in part on detection of the second adaptor nucleic acid sequence in the second plurality of sequence reads. In some embodiments, the first plurality of sequence reads is at least 2×, at least 4×, at least 8×, at least 10×, at least 50×, at least 100×, or at least 1000× more abundant than the second plurality of sequence reads. In some embodiments, the second plurality of sequence reads is at least 2×, at least 4×, at least 8×, at least 10×, at least 50×, at least 100×, or at least 1000× more abundant than the first plurality of sequence reads. In some embodiments, the first and/or second plurality of sequence reads is obtained by sequencing; optionally wherein the sequencing comprises use of a massively parallel sequencing (MPS) technique, whole genome sequencing (WGS), whole exome sequencing, targeted sequencing, direct sequencing, or a Sanger sequencing technique; and optionally wherein the massively parallel sequencing technique comprises next generation sequencing (NGS). In some embodiments, the method further comprises generating, based at least in part on the analyzing, a molecular profile for the sample. In some embodiments, the molecular profile further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the individual is administered a treatment based at least in part on the molecular profile. In some embodiments, the molecular profile further comprises results from a nucleic acid sequencing-based test. In some embodiments, the method further comprises comparing a sequence read of the second plurality of sequence reads with a sequence read of the first plurality of sequence reads. In some embodiments, the method further comprises comparing one or more sequence read(s) of the first and/or second plurality of sequence reads with a reference genome sequence.

In some embodiments, a molecular profile or report of the present disclosure comprises results from sequencing the methylation strand and/or genomic strand, e.g., using the methods of the present disclosure.

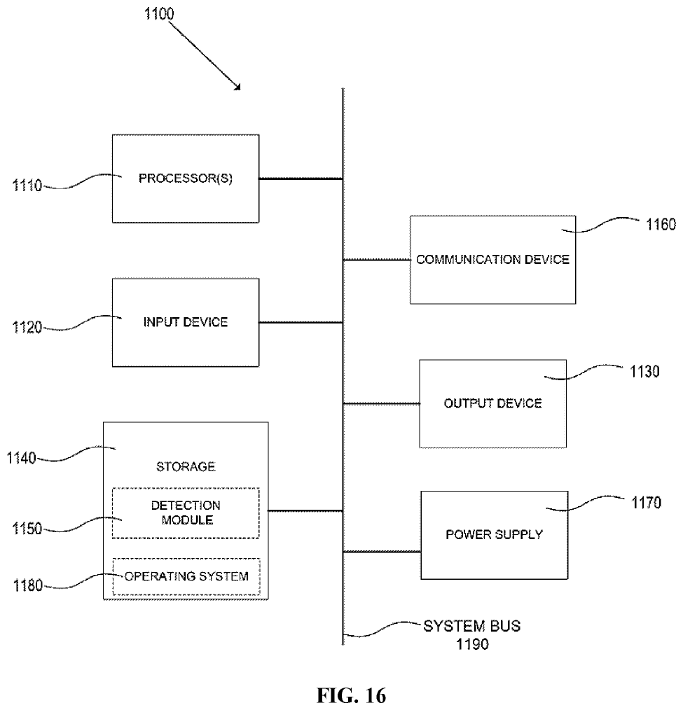

FIG. 16 illustrates an example of a computing device in accordance with one embodiment. Device 1100 can be a host computer connected to a network. Device 1100 can be a client computer or a server. As shown in FIG. 16, device 1100 can be any suitable type of microprocessor-based device, such as a personal computer, workstation, server or handheld computing device (portable electronic device) such as a phone or tablet. The device can include, for example, one or more of processor(s) 1110, input device 1120, output device 1130, storage 1140, communication device 1160, power supply 1170, operating system 1180, and system bus 1190. Input device 1120 and output device 1130 can generally correspond to those described herein, and can either be connectable or integrated with the computer.

Input device 1120 can be any suitable device that provides input, such as a touch screen, keyboard or keypad, mouse, or voice-recognition device. Output device 1130 can be any suitable device that provides output, such as a touch screen, haptics device, or speaker.

Storage 1140 can be any suitable device that provides storage (e.g., an electrical, magnetic or optical memory including a RAM (volatile and non-volatile), cache, hard drive, or removable storage disk). Communication device 1160 can include any suitable device capable of transmitting and receiving signals over a network, such as a network interface chip or device. The components of the computer can be connected in any suitable manner, such as via a wired media (e.g., a physical bus, ethernet, or any other wire transfer technology) or wirelessly (e.g., Bluetooth®, Wi-FiR, or any other wireless technology). For example, in FIG. 16, the components are connected by System Bus 1190.

Detection module 1150, which can be stored as executable instructions in storage 1140 and executed by processor(s) 1110, can include, for example, the processes that embody the functionality of the present disclosure (e.g., as embodied in the devices as described herein).

Detection module 1150 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 1140, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Detection module 1150 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

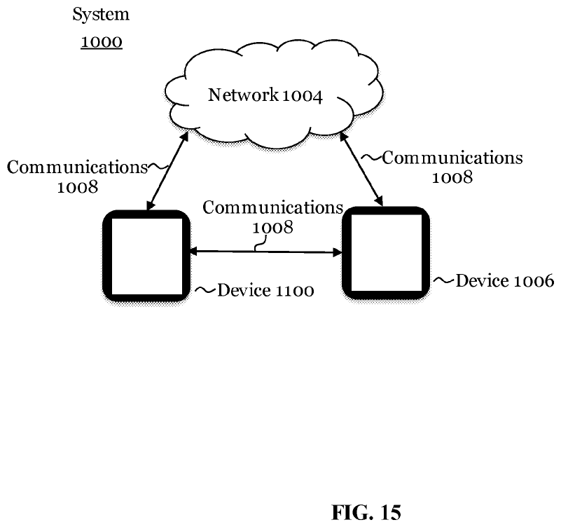

Device 1100 may be connected to a network (e.g., Network 1004, as shown in FIG. 15 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 1100 can implement any operating system (e.g., Operating System 1180) suitable for operating on the network. Detection module 1150 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, Operating System 1180 is executed by one or more processors, e.g., Processor(s) 1110.

Device 1100 can further include Power Supply 1170, which can be any suitable power supply.

In some embodiments, Detection module 1150 is a module for detecting LOH of one or more HLA-I genes and/or tumor mutational burden and includes the processes that embody the functionality of the present disclosure (e.g., as embodied in the devices as described herein).

FIG. 15 illustrates an example of a computing system in accordance with one embodiment. In System 1000, Device 1100 (e.g., as described above and illustrated in FIG. 16) is connected to Network 1004, which is also connected to Device 1006. In some embodiments, Device 1006 is a sequencer. Exemplary sequencers can include, without limitation, Roche/454's Genome Sequencer (GS) FLX System. Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio RS system. Devices 1100 and 1006 may communicate, e.g., using suitable communication interfaces via Network 1004, such as a Local Area Network (LAN), Virtual Private Network (VPN), or the Internet. In some embodiments, Network 1004 can be, for example, the Internet, an intranet, a virtual private network, a cloud network, a wired network, or a wireless network. Devices 1100 and 1006 may communicate, in part or in whole, via wireless or hardwired communications, such as Ethernet, IEEE 802.11b wireless, or the like. Additionally, Devices 1100 and 1006 may communicate, e.g., using suitable communication interfaces, via a second network, such as a mobile/cellular network. Communication between Devices 1100 and 1006 may further include or communicate with various servers such as a mail server, mobile server, media server, telephone server, and the like. In some embodiments, Devices 1100 and 1006 can communicate directly (instead of, or in addition to, communicating via Network 1004), e.g., via wireless or hardwired communications, such as Ethernet, IEEE 802.11b wireless, or the like. In some embodiments, Devices 1100 and 1006 communicate via Communications 1008, which can be a direct connection or can occur via a network (e.g., Network 1004).

One or all of Devices 1100 and 1006 generally include logic (e.g., http web server logic) or is programmed to format data, accessed from local or remote databases or other sources of data and content, for providing and/or receiving information via Network 1004 according to various examples described herein.

FIG. 14C illustrates an exemplary process 1400 for detecting genetic and epigenetic information in a single workflow, in accordance with some embodiments of the present disclosure. Process 1400 is performed, for example, using one or more electronic devices implementing a software program. In some examples, process 1400 is performed using a client-server system, and the blocks of process 1400 are divided up in any manner between the server and a client device. In other examples, the blocks of process 1400 are divided up between the server and multiple client devices. Thus, while portions of process 1400 are described herein as being performed by particular devices of a client-server system, it will be appreciated that process 1400 is not so limited. In some embodiments, the executed steps can be executed across many systems, e.g., in a cloud environment. In other examples, process 1400 is performed using only a client device or only multiple client devices. In process 1400, some blocks are, optionally, combined, the order of some blocks is, optionally, changed, and some blocks are, optionally, omitted. In some examples, additional steps may be performed in combination with the process 1400. Accordingly, the operations as illustrated (and described in greater detail below) are exemplary by nature and, as such, should not be viewed as limiting.

At block 1402, a first plurality of sequence reads of one or more first nucleic acid molecules or their amplification products is obtained, wherein the first nucleic acid molecules have undergone cytosine conversion treatment under conditions (e.g., as described herein) such that unmethylated cytosine(s) if present in the first nucleic acid molecules have undergone cytosine conversion.

At block 1404, a second plurality of sequence reads of one or more second nucleic acid molecules or their amplification products is obtained, wherein the second nucleic acid molecules are complementary to the first nucleic acid molecules prior to cytosine conversion and comprise a cytosine analog that is resistant to cytosine conversion (e.g., as described herein).

At block 1406, an exemplary system (e.g., one or more electronic devices) analyzes the first plurality of sequence reads for the presence or absence of methylation at one or more cytosines inferred based on cytosine conversion or a lack thereof.

At block 1408, an exemplary system (e.g., one or more electronic devices) analyzes the second plurality of sequence reads for sequence information.

Optionally, the method further comprises demultiplexing the first and second pluralities of sequence reads based at least in part on detection of the first adaptor nucleic acid sequence in the first plurality of sequence reads.

In some embodiments, the first and/or second pluralities of sequence reads are obtained using a sequencer, e.g., as described herein or otherwise known in the art, such as that for performing WGMS and/or WGS.

In some embodiments, prior to blocks 1402 and/or 1402, the first and/or second pluralities of sequence reads are generated by any of the methods of the present disclosure, e.g., based on methylation and/or genomic strands as described herein.

### Reporting

In some embodiments, the methods provided herein comprise generating a report, and/or providing a report to party. In some embodiments, the report comprises one or more treatment options identified for the individual, e.g., based at least in part on methylation and/or somatic mutations or lack thereof detected in a sample from the individual as described herein. In some embodiments, the one or more treatment options are based at least in part on methylation and/or somatic mutation(s) detected or not detected.

A report according to the present disclosure may be in an electronic, web-based, or paper form. The report may be provided to an individual or a patient (e.g., an individual or a patient with a cancer), or to an individual or entity other than the individual or patient (e.g., other than the individual or patient with the cancer), such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from detecting methylation and/or somatic mutation(s) in a sample obtained from an individual (e.g., an individual having a cancer).

### IV. Exemplary Embodiments

The following exemplary embodiments are representative of some aspects of the invention:

Embodiment 1. A method of detecting genetic and epigenetic information in a single workflow, comprising:
providing a plurality of first single-stranded DNA fragments;
subjecting the plurality of first single-stranded DNA fragments to a round of primer extension in the presence of: (a) a primer that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands corresponding to the first single-stranded DNA fragments and a plurality of second strands that are complementary to the first strands, wherein the second strands comprise the cytosine analog that is resistant to cytosine conversion;
subjecting the pluralities of the first and second strands to a cytosine conversion treatment under conditions such that unmethylated cytosine(s) if present in the first strands undergo cytosine conversion; and
detecting at least a portion of the plurality of first strands and at least a portion of the plurality of second strands.

Embodiment 2. The method of embodiment 1, wherein the detecting is by sequencing.

Embodiment 3. The method of embodiment 2, wherein the sequencing is next-generation sequencing (NGS).

Embodiment 4. The method of embodiment 1, wherein the detecting is by microarray, quantitative PCR (qPCR), digital droplet PCR (ddPCR), or molecular inversion probes.

Embodiment 5. The method of any one of embodiments 1-4, wherein the method further comprises, prior to detecting: subjecting the plurality of first strands and/or the plurality of second strands to amplification, wherein the detecting comprises detecting at least a portion of the pluralities of first and second strands and their amplification products if present.

Embodiment 6. The method of embodiment 5, comprising subjecting the plurality of second strands to amplification in the presence of one or more primer(s) that anneal with at least a portion of the plurality of second strands, but not with the plurality of first strands.

Embodiment 7. The method of embodiment 5 or embodiment 6, comprising subjecting the plurality of first strands to amplification in the presence of one or more primer(s) that anneal with at least a portion of the plurality of first strands, but not with the plurality of second strands.

Embodiment 8. The method of embodiment 7, wherein the one or more primer(s) anneal with at least a portion of the plurality of first strands after the first strands undergo cytosine conversion, but not before the first strands undergo cytosine conversion.

Embodiment 9. The method of embodiment 7, wherein the one or more primer(s) anneal with at least a portion of the plurality of first strands only if cytosines of the first strands do not undergo cytosine conversion.

Embodiment 10. The method of any one of embodiments 5-9, wherein the amplification occurs after cytosine conversion.

Embodiment 11. The method of any one of embodiments 1-10, wherein the method further comprises, prior to detecting: enriching for the plurality of second strands or their amplification products.

Embodiment 12. The method of any one of embodiments 1-11, wherein the method further comprises, prior to detecting: enriching for the plurality of first strands or their amplification products.

Embodiment 13. The method of embodiment 11 or embodiment 12, wherein the method comprises, prior to detecting: separating one or more first strands or their amplification products from one or more second strands or their amplification products.

Embodiment 14. The method of embodiment 13, wherein the separation comprises: (a) combining one or more bait molecules with the pluralities of first and second strands, wherein the one or more bait molecules preferentially hybridize with one or more of the first strands or their amplification products, or with one or more of the second strands or their amplification products, thereby producing nucleic acid hybrids; and (b) isolating the nucleic acid hybrids.

Embodiment 15. The method of embodiment 14, wherein the one or more bait molecules hybridize with one or more of the first strands or their amplification products but not with the plurality of second strands or their amplification products.

Embodiment 16. The method of embodiment 15, wherein the separation occurs after the cytosine conversion treatment, and wherein the one or more bait molecules hybridize with one or more of the first strands or their amplification products if one or more cytosines of the first strands undergo cytosine conversion.

Embodiment 17. The method of embodiment 14, wherein the one or more bait molecules hybridize with one or more of the second strands or their amplification products but not with the plurality of first strands or their amplification products.

Embodiment 18. The method of embodiment 13, wherein the separation comprises:
(a) combining one or more first bait molecules with the pluralities of first and second strands, wherein the one or more first bait molecules preferentially hybridize with one or more of the first strands or their amplification products, thereby producing first nucleic acid hybrids;
(b) isolating the first nucleic acid hybrids;
(c) combining one or more second bait molecules with the pluralities of first and second strands, wherein the one or more second bait molecules preferentially hybridize with one or more of the second strands or their amplification products, thereby producing second nucleic acid hybrids; and
(d) isolating the second nucleic acid hybrids.

Embodiment 19. The method of any one of embodiments 1-18, wherein the pluralities of first and second strands or their amplification products are detected together.

Embodiment 20. The method of any one of embodiments 1-18, wherein the pluralities of first and second strands or their amplification products are detected separately.

Embodiment 21. The method of any one of embodiments 1-20, wherein the method comprises subjecting the plurality of first single-stranded DNA fragments to two or more rounds of primer extension prior to cytosine conversion in the presence of: (a) a primer that anneals to at least a portion of the first single-stranded DNA fragments, (b) a nucleic acid polymerase, and (c) a mixture of nucleotides comprising a cytosine analog that is resistant to cytosine conversion.

Embodiment 22. The method of any one of embodiments 1-21, further comprising attaching one or more nucleic acid adaptors to one or more of the first single-stranded DNA fragments.

Embodiment 23. The method of any one of embodiments 1-22, further comprising attaching one or more nucleic acid adaptors to one or more of the second strands.

Embodiment 24. The method of embodiment 22 or embodiment 23, wherein the one or more nucleic acid adaptors are attached to the first or the second strands by ligation, transposition, tailing, or template switching.

Embodiment 25. A method of detecting genetic and epigenetic information in a single workflow, comprising:
providing a plurality of first single-stranded DNA fragments;
attaching a first adaptor nucleic acid onto at least a portion of the first single-stranded DNA fragments of the plurality;
subjecting the plurality of first single-stranded DNA fragments to a round of primer extension in the presence of: (a) a primer that anneals to at least a portion of the first single-stranded DNA fragments downstream or at the first adaptor nucleic acid, (b) a nucleic acid polymerase, and (c) a mixture of nucleotides comprising a cytosine analog that is resistant to cytosine conversion, thereby generating a plurality of first strands comprising the first single-stranded DNA fragments with the first adaptor nucleic acid and a plurality of second strands comprising second single-stranded DNA fragments complementary to the first single-stranded DNA fragments and a second adaptor nucleic acid complementary to the first adaptor nucleic acid, wherein the second strands comprise the cytosine analog that is resistant to cytosine conversion;
subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and
detecting at least a portion of the plurality of first strands and at least a portion of the plurality of second strands.

Embodiment 26. The method of embodiment 25, wherein the first adaptor nucleic acid is attached onto 3′ end(s) of at least a portion of the first single-stranded DNA fragments of the plurality.

Embodiment 27. The method of embodiment 25, wherein the first adaptor nucleic acid is attached onto 5′ end(s) of at least a portion of the first single-stranded DNA fragments of the plurality.

Embodiment 28. The method of any one of embodiments 25-27, wherein the first adaptor nucleic acid comprises one or more unmethylated cytosines that are converted during cytosine conversion.

Embodiment 29. The method of any one of embodiments 25-27, wherein the first adaptor nucleic acid comprises one or more methylated cytosines, and wherein the primer comprises one or more unmethylated cytosines that are converted during cytosine conversion.

Embodiment 30. A method of detecting genetic and epigenetic information in a single workflow, comprising:
providing a plurality of first single-stranded DNA fragments;
subjecting the plurality of first single-stranded DNA fragments to a round of primer extension in the presence of: (a) a primer comprising: (i) a second adaptor nucleic acid portion that does not anneal to the first single-stranded DNA fragments and (ii) a portion that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands comprising the first single-stranded DNA fragments with a first adaptor nucleic acid complementary to the second adaptor nucleic acid and a plurality of second strands comprising second single-stranded DNA fragments complementary to the first single-stranded DNA fragments and the second adaptor nucleic acid non-complementary to the first adaptor nucleic acid, wherein the second strands comprise the cytosine analog that is resistant to cytosine conversion;
subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and
detecting at least a portion of the plurality of first strands and at least a portion of the plurality of second strands.

Embodiment 31. The method of embodiment 30, wherein the second adaptor nucleic acid portion of the primer is 5′ relative to the portion of the primer that anneals to the first single-stranded DNA fragments.

Embodiment 32. The method of embodiment 31, wherein the primer comprises one or more unmethylated cytosines that become part of the second adaptor nucleic acid after primer extension and are converted during cytosine conversion.

Embodiment 33. A method of detecting genetic and epigenetic information in a single workflow, comprising:
providing a plurality of first single-stranded DNA fragments;
subjecting the plurality of first single-stranded DNA fragments to a round of primer extension in the presence of: (a) a primer comprising: (i) a second adaptor nucleic acid portion comprising a non-complementary 5′ overhang that does not anneal to the first single-stranded DNA fragments, and (ii) a portion that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands comprising the first single-stranded DNA fragments with a first adaptor nucleic acid complementary to the portion of the second adaptor nucleic acid that anneals to the first single-stranded DNA fragments and a plurality of second strands comprising second single-stranded DNA fragments complementary to the first single-stranded DNA fragments and the second adaptor nucleic acid, wherein the second strands comprise the cytosine analog that is resistant to cytosine conversion;
subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and
detecting at least a portion of the plurality of first strands and at least a portion of the plurality of second strands.

Embodiment 34. The method of any one of embodiments 25-33, wherein the detecting is by sequencing.

Embodiment 35. The method of embodiment 34, wherein the sequencing is next-generation sequencing (NGS).

Embodiment 36. The method of any one of embodiments 25-33, wherein the detecting is by microarray, quantitative PCR (qPCR), digital droplet PCR (ddPCR), or molecular inversion probes.

Embodiment 37. The method of any one of embodiments 25-36, further comprising: demultiplexing sequence information from the first and second strands based on the first and/or second adaptor nucleic acids.

Embodiment 38. The method of any one of embodiments 25-37, wherein the method further comprises, prior to detecting: subjecting the plurality of first strands and/or the plurality of second strands to amplification, wherein the detecting comprises detecting at least a portion of the pluralities of first and second strands and their amplification products if present.

Embodiment 39. The method of embodiment 38, comprising subjecting the plurality of second strands to amplification in the presence of one or more primer(s) that anneal with at least a portion of the plurality of second strands, but not with the plurality of first strands.

Embodiment 40. The method of embodiment 39, wherein the one or more primer(s) that anneal with at least a portion of the plurality of second strands anneal with at least a portion of the second adaptor nucleic acid.

Embodiment 41. The method of any one of embodiments 38-40, comprising subjecting the plurality of first strands to amplification in the presence of one or more primer(s) that anneal with at least a portion of the plurality of first strands, but not with the plurality of second strands.

Embodiment 42. The method of embodiment 41, wherein the one or more primer(s) that anneal with at least a portion of the plurality of first strands anneal with at least a portion of the first adaptor nucleic acid.

Embodiment 43. The method of embodiment 41, wherein the one or more primer(s) anneal with at least a portion of the plurality of first strands after the first strands undergo cytosine conversion, but not before the first strands undergo cytosine conversion.

Embodiment 44. The method of embodiment 41, wherein the one or more primer(s) anneal with at least a portion of the plurality of first strands only if cytosines of the first strands do not undergo cytosine conversion.

Embodiment 45. The method of any one of embodiments 38-44, wherein the amplification occurs after cytosine conversion.

Embodiment 46. The method of any one of embodiments 25-45, wherein the method further comprises, prior to detecting: enriching for the plurality of second strands or their amplification products.

Embodiment 47. The method of any one of embodiments 25-46, wherein the method further comprises, prior to detecting: enriching for the plurality of first strands or their amplification products.

Embodiment 48. The method of embodiment 46 or embodiment 47, wherein the method comprises, prior to detecting: separating one or more first strands or their amplification products from one or more second strands or their amplification products.

Embodiment 49. The method of embodiment 48, wherein the separation comprises: (a) combining one or more bait molecules with the pluralities of first and second strands, wherein the one or more bait molecules preferentially hybridize with one or more of the first strands or their amplification products, or with one or more of the second strands or their amplification products, thereby producing nucleic acid hybrids; and (b) isolating the nucleic acid hybrids.

Embodiment 50. The method of embodiment 49, wherein the one or more bait molecules hybridize with one or more of the first strands or their amplification products but not with the plurality of second strands or their amplification products.

Embodiment 51. The method of embodiment 49 or embodiment 50, wherein the one or more bait molecules hybridize with at least a portion of the first adaptor nucleic acid.

Embodiment 52. The method of embodiment 49 or embodiment 50, wherein the separation occurs after the cytosine conversion treatment, and wherein the one or more bait molecules hybridize with one or more of the first strands or their amplification products if one or more cytosines of the first strands undergo cytosine conversion.

Embodiment 53. The method of embodiment 49, wherein the one or more bait molecules hybridize with one or more of the second strands or their amplification products but not with the plurality of first strands or their amplification products.

Embodiment 54. The method of embodiment 53, wherein the one or more bait molecules hybridize with at least a portion of the second adaptor nucleic acid.

Embodiment 55. The method of embodiment 48, wherein the separation comprises:
(a) combining one or more first bait molecules with the pluralities of first and second strands, wherein the one or more first bait molecules preferentially hybridize with one or more of the first strands or their amplification products, thereby producing first nucleic acid hybrids;
(b) isolating the first nucleic acid hybrids;
(c) combining one or more second bait molecules with the pluralities of first and second strands, wherein the one or more second bait molecules preferentially hybridize with one or more of the second strands or their amplification products, thereby producing second nucleic acid hybrids; and
(d) isolating the second nucleic acid hybrids.

Embodiment 56. The method of embodiment 55, wherein the one or more first bait molecules hybridize with at least a portion of the first adaptor nucleic acid, and/or wherein the one or more second bait molecules hybridize with at least a portion of the second adaptor nucleic acid.

Embodiment 57. The method of any one of embodiments 25-56, wherein the pluralities of first and second strands or their amplification products are detected together.

Embodiment 58. The method of any one of embodiments 25-56, wherein the pluralities of first and second strands or their amplification products are detected separately.

Embodiment 59. The method of any one of embodiments 25-58, wherein the method comprises subjecting the plurality of first single-stranded DNA fragments to two or more rounds of primer extension prior to cytosine conversion in the presence of: (a) a primer that anneals to at least a portion of the first single-stranded DNA fragments, (b) a nucleic acid polymerase, and (c) a mixture of nucleotides comprising a cytosine analog that is resistant to cytosine conversion.

Embodiment 60. The method of any one of embodiments 1, 2, 5-34, and 37-59, wherein the plurality of first strands is sequenced at a different depth of sequencing than the plurality of second strands.

Embodiment 61. The method of embodiment 60, wherein the plurality of second strands is sequenced at a depth of sequencing that is at least 2×, at least 4×, at least 8×, at least 10×, at least 50×, at least 100×, or at least 1000× higher than depth of sequencing of the plurality of first strands.

Embodiment 62. The method of embodiment 60, wherein the plurality of first strands is sequenced at a depth of sequencing that is at least 2×, at least 4×, at least 8×, at least 10×, at least 50×, at least 100×, or at least 1000× higher than depth of sequencing of the plurality of second strands.

Embodiment 63. The method of any one of embodiments 1-62, wherein, after primer extension, the plurality of first strands is hybridized with the plurality of second strands in a plurality of double-stranded nucleic acids.

Embodiment 64. The method of any one of embodiments 1-63, further comprising, prior to the round of primer extension: denaturing a plurality of double-stranded DNA fragments to provide the plurality of first single-stranded DNA fragments.

Embodiment 65. The method of any one of embodiments 1-64, further comprising obtaining the plurality of single- or double-stranded DNA fragments from a sample.

Embodiment 66. The method of embodiment 65, further comprising obtaining the sample from an individual.

Embodiment 67. The method of embodiment 66, wherein the individual has, is suspected to have, or is being treated for cancer.

Embodiment 68. The method of embodiment 66, wherein the individual is being screened for cancer or a recurrence of cancer.

Embodiment 69. The method of any one of embodiments 65-68, wherein the sample comprises tissue, cells, and/or nucleic acids from a cancer.

Embodiment 70. The method of any one of embodiments 65-69, wherein the sample comprises tissue, cells, and/or nucleic acids from normal tissue.

Embodiment 71. The method of any one of embodiments 65-70, wherein the sample comprises a tissue biopsy sample, a liquid biopsy sample, or a normal control.

Embodiment 72. The method of embodiment 71, wherein the sample is from a tumor biopsy, tumor specimen, or circulating tumor cell.

Embodiment 73. The method of any one of embodiments 65-70, wherein the sample is a liquid biopsy sample and comprises blood, plasma, serum, cerebrospinal fluid, sputum, stool, urine, or saliva

Embodiment 74. The method of any one of embodiments 1-73, wherein the cytosine analog that is resistant to cytosine conversion comprises 5-methylcytosine (5mC), 5-hydroxymethylcytosine (5hmC), 5-carboxylcytosine (5caC), 5-formylcytosine, 5-(beta-D-glucosylmethyl) cytosine (5gmC), 5-ethyl dCTP, 5-methyl dCTP, 5-fluoro dCTP, 5-bromo dCTP, 5-iodo dCTP, 5-chloro dCTP, 5-trifluoromethyl dCTP, or 5-aza dCTP.

Embodiment 75. The method of any one of embodiments 1-74, wherein the cytosine conversion is by bisulfite treatment, TET-assisted bisulfite treatment, oxidative bisulfite treatment, APOBEC, or TET/beta-glucosyltransferase assisted APOBEC treatment.

Embodiment 76. The method of any one of embodiments 1-75, wherein at least 80%, at least 85%, at least 90%, at least 95%, or 100% of unmethylated cytosine(s) if present in the first strands undergo cytosine conversion as a result of the cytosine conversion treatment.

Embodiment 77. The method of any one of embodiments 1-76, wherein between about 80% and about 97% of unmethylated cytosine(s) if present in the first strands undergo cytosine conversion as a result of the cytosine conversion treatment.

Embodiment 78. The method of any one of embodiments 1-77, wherein at most 20%, at most 15%, at most 10%, at most 5%, at most 2%, at most 1%, or at most 0.5% of cytosine analogs of the second strands undergo cytosine conversion as a result of the cytosine conversion treatment.

Embodiment 79. The method of any one of embodiments 1-78, wherein between about 0.5% and about 5% of cytosine analogs of the second strands undergo cytosine conversion as a result of the cytosine conversion treatment.

Embodiment 80. The method of any one of embodiments 1-79, wherein the nucleic acid polymerase is capable of incorporating the cytosine analog into nucleic acid.

Embodiment 81. The method of any one of embodiments 1-80, further comprising, prior to primer extension: subjecting the plurality of first single-stranded DNA fragments to end repair.

Embodiment 82. The method of any one of embodiments 1, 2, 5-34, and 37-81, further comprising, after sequencing the pluralities of first and second strands or their amplification products: comparing a sequence of the plurality of the second strands with a sequence of the plurality of the first strands.

Embodiment 83. The method of any one of embodiments 1, 2, 5-34, and 37-81, further comprising, after sequencing the pluralities of first and second strands or their amplification products: comparing a sequence of the plurality of the first and/or the second strands with a reference genome sequence.

Embodiment 84. A method of detecting cancer in an individual, comprising detecting methylation level and/or somatic mutation(s) according to the method of any one of embodiments 1-83 in a sample comprising a plurality of nucleic acids obtained from the individual, wherein the methylation level and/or somatic mutation(s) detected in the sample identify the individual as having cancer.

Embodiment 85. A method of detecting minimal residual disease in an individual who has been treated or is undergoing treatment for cancer, comprising detecting methylation level and/or somatic mutation(s) according to the method of any one of embodiments 1-83 in a sample comprising a plurality of nucleic acids obtained from the individual, wherein the methylation level and/or somatic mutation(s) detected in the sample identify the individual as having minimal residual disease or a lack thereof.

Embodiment 86. A method of screening an individual suspected of having cancer, comprising detecting methylation level and/or somatic mutation(s) according to the method of any one of embodiments 1-83 in a sample comprising a plurality of nucleic acids obtained from the individual, wherein the methylation level and/or somatic mutation(s) detected in the sample identify the individual as likely to have cancer.

Embodiment 87. A method of determining prognosis of an individual having cancer, comprising detecting methylation level and/or somatic mutation(s) according to the method of any one of embodiments 1-83 in a sample comprising a plurality of nucleic acids obtained from the individual, wherein the methylation level and/or somatic mutation(s) detected in the sample determine at least in part the prognosis of the individual.

Embodiment 88. A method of predicting survival of an individual having cancer, comprising detecting methylation level and/or somatic mutation(s) according to the method of any one of embodiments 1-83 in a sample comprising a plurality of nucleic acids obtained from the individual, wherein the methylation level and/or somatic mutation(s) detected in the sample predict at least in part the survival of the individual.

Embodiment 89. A method of predicting or detecting tumor burden of an individual having cancer, comprising detecting methylation level and/or somatic mutation(s) according to the method of any one of embodiments 1-83 in a sample comprising a plurality of nucleic acids obtained from the individual, wherein the methylation level and/or somatic mutation(s) detected in the sample predict or detect at least in part the tumor burden of the individual.

Embodiment 90. A method of predicting responsiveness to treatment of an individual having cancer, comprising detecting methylation level and/or somatic mutation(s) according to the method of any one of embodiments 1-83 in a sample comprising a plurality of nucleic acids obtained from the individual, wherein the methylation level and/or somatic mutation(s) detected in the sample are used at least in part to predict responsiveness of the individual to a treatment.

Embodiment 91. A method of monitoring response of an individual being treated for cancer, comprising:
(a) administering a treatment to an individual having cancer; and
(b) detecting methylation level and/or somatic mutation(s) according to the method of any one of embodiments 1-83 in a sample comprising a plurality of nucleic acids obtained from the individual; wherein the methylation level and/or somatic mutation(s) detected in the sample are used at least in part to monitor response to the treatment.

Embodiment 92. A method of monitoring a cancer in an individual, comprising:
(a) detecting methylation level and/or somatic mutation(s) according to the method of any one of embodiments 1-83 in a first sample comprising a plurality of nucleic acids obtained from the individual;
(b) detecting methylation level and/or somatic mutation(s) according to the method of any one of embodiments 1-83 in a second sample comprising a plurality of nucleic acids obtained from the individual; and
(c) determining a difference in methylation level and/or somatic mutation(s) between the first and second samples, thereby monitoring the cancer in the individual.

Embodiment 93. A system, comprising:
one or more processors; and
a memory configured to store one or more computer program instructions, wherein the one or more computer program instructions when executed by the one or more processors are configured to:
   obtain a first plurality of sequence reads of one or more first nucleic acid molecules or their amplification products, wherein the first nucleic acid molecules have undergone cytosine conversion treatment under conditions such that unmethylated cytosine(s) if present in the first nucleic acid molecules have undergone cytosine conversion;
   obtain a second plurality of sequence reads of one or more second nucleic acid molecules or their amplification products, wherein the second nucleic acid molecules are complementary to the first nucleic acid molecules prior to cytosine conversion and comprise a cytosine analog that is resistant to cytosine conversion;
   analyze the first plurality of sequence reads for the presence or absence of methylation at one or more cytosines inferred based on cytosine conversion or a lack thereof; and
   analyze the second plurality of sequence reads for sequence information.

Embodiment 94. The system of embodiment 93, wherein the one or more first nucleic acid molecules or their amplification products further comprise a first adaptor nucleic acid sequence, and wherein the one or more computer program instructions when executed by the one or more processors are further configured to: demultiplex the first and second pluralities of sequence reads based at least in part on detection of the first adaptor nucleic acid sequence in the first plurality of sequence reads.

Embodiment 95. The system of embodiment 94, wherein the first adaptor nucleic acid is attached onto 3′ end(s) of at least a portion of the first nucleic acid molecules of the plurality.

Embodiment 96. The system of embodiment 94, wherein the first adaptor nucleic acid is attached onto 5′ end(s) of at least a portion of the first nucleic acid molecules of the plurality.

Embodiment 97. The system of any one of embodiments 94-96, wherein the first adaptor nucleic acid comprises one or more unmethylated cytosines that are converted during cytosine conversion.

Embodiment 98. The system of any one of embodiments 94-96, wherein the first adaptor nucleic acid comprises one or more methylated cytosines.

Embodiment 99. The system of embodiment 94, wherein the first adaptor nucleic acid is between 5′ and 3′ ends of at least a portion of the first nucleic acid molecules of the plurality.

Embodiment 100. The system of any one of embodiments 93-99, wherein the one or more second nucleic acid molecules or their amplification products further comprise a second adaptor nucleic acid sequence, and wherein the one or more computer program instructions when executed by the one or more processors are further configured to: demultiplex the first and second pluralities of sequence reads based at least in part on detection of the second adaptor nucleic acid sequence in the second plurality of sequence reads.

Embodiment 101. The system of embodiment 100, wherein the second adaptor nucleic acid is attached onto 3′ end(s) of at least a portion of the second nucleic acid molecules of the plurality.

Embodiment 102. The system of embodiment 100, wherein the second adaptor nucleic acid is attached onto 5′ end(s) of at least a portion of the second nucleic acid molecules of the plurality.

Embodiment 103. The system of embodiment 100, wherein the second adaptor nucleic acid is between 5′ and 3′ ends of at least a portion of the second nucleic acid molecules of the plurality.

Embodiment 104. The system of any one of embodiments 94-103, wherein the first plurality of sequence reads is at least 2×, at least 4×, at least 8×, at least 10×, at least 50×, at least 100×, or at least 1000× more abundant than the second plurality of sequence reads.

Embodiment 105. The system of any one of embodiments 94-103, wherein the second plurality of sequence reads is at least 2×, at least 4×, at least 8×, at least 10×, at least 50×, at least 100×, or at least 1000× more abundant than the first plurality of sequence reads.

Embodiment 106. The system of any one of embodiments 94-105, wherein the first nucleic acid molecules are obtained from a sample prior to cytosine conversion.

Embodiment 107. The system of embodiment 106, wherein the sample is from an individual having or suspected of having a cancer.

Embodiment 108. The system of embodiment 107, wherein the sample comprises tissue, cells, and/or nucleic acids from a cancer.

Embodiment 109. The system of embodiment 107 or embodiment 108, wherein the sample comprises tissue, cells, and/or nucleic acids from normal tissue.

Embodiment 110. The system of any one of embodiments 106-109, wherein the first and/or second plurality of sequence reads is obtained by sequencing; optionally wherein the sequencing comprises use of a massively parallel sequencing (MPS) technique, whole genome sequencing (WGS), whole exome sequencing, targeted sequencing, direct sequencing, or a Sanger sequencing technique; and optionally wherein the massively parallel sequencing technique comprises next generation sequencing (NGS).

Embodiment 111. The system of any one of embodiments 106-110, wherein the one or more program instructions when executed by the one or more processors are further configured to generate, based at least in part on the analyzing, a molecular profile for the sample.

Embodiment 112. The system of embodiment 111, wherein a treatment is administered to an individual based at least in part on the molecular profile.

Embodiment 113. The system of embodiment 111 or embodiment 112, wherein the molecular profile further comprises results from a comprehensive genomic profiling (CGP) test, a gene expression profiling test, a cancer hotspot panel test, a DNA methylation test, a DNA fragmentation test, an RNA fragmentation test, or any combination thereof.

Embodiment 114. The system of any one of embodiments 111-113, wherein the molecular profile further comprises results from a nucleic acid sequencing-based test.

Embodiment 115. The system of any one of embodiments 94-114, wherein the one or more computer program instructions when executed by the one or more processors are further configured to: compare a sequence read of the second plurality of sequence reads with a sequence read of the first plurality of sequence reads.

Embodiment 116. The system of any one of embodiments 94-114, wherein the one or more computer program instructions when executed by the one or more processors are further configured to: compare one or more sequence read(s) of the first and/or second plurality of sequence reads with a reference genome sequence.

Embodiment 117. A non-transitory computer readable storage medium comprising one or more programs executable by one or more computer processors for performing a method, comprising:
obtaining a first plurality of sequence reads of one or more first nucleic acid molecules or their amplification products, wherein the first nucleic acid molecules have undergone cytosine conversion treatment under conditions such that unmethylated cytosine(s) if present in the first nucleic acid molecules have undergone cytosine conversion;
obtaining a second plurality of sequence reads of one or more second nucleic acid molecules or their amplification products, wherein the second nucleic acid molecules are complementary to the first nucleic acid molecules prior to cytosine conversion and comprise a cytosine analog that is resistant to cytosine conversion;
analyzing the first plurality of sequence reads for the presence or absence of methylation at one or more cytosines inferred based on cytosine conversion or a lack thereof; and
analyzing the second plurality of sequence reads for sequence information.

Embodiment 118. The non-transitory computer readable storage medium of embodiment 117, wherein the one or more first nucleic acid molecules or their amplification products further comprise a first adaptor nucleic acid sequence, and the method further comprises: demultiplexing the first and second pluralities of sequence reads based at least in part on detection of the first adaptor nucleic acid sequence in the first plurality of sequence reads.

Embodiment 119. The non-transitory computer readable storage medium of embodiment 118, wherein the first adaptor nucleic acid is attached onto 3′ end(s) of at least a portion of the first nucleic acid molecules of the plurality.

Embodiment 120. The non-transitory computer readable storage medium of embodiment 118, wherein the first adaptor nucleic acid is attached onto 5′ end(s) of at least a portion of the first nucleic acid molecules of the plurality.

Embodiment 121. The non-transitory computer readable storage medium of any one of embodiments 118-120, wherein the first adaptor nucleic acid comprises one or more unmethylated cytosines that are converted during cytosine conversion.

Embodiment 122. The non-transitory computer readable storage medium of any one of embodiments 118-120, wherein the first adaptor nucleic acid comprises one or more methylated cytosines.

Embodiment 123. The non-transitory computer readable storage medium of embodiment 118, wherein the first adaptor nucleic acid is between 5′ and 3′ ends of at least a portion of the first nucleic acid molecules of the plurality.

Embodiment 124. The non-transitory computer readable storage medium of any one of embodiments 117-123, wherein the one or more second nucleic acid molecules or their amplification products further comprise a second adaptor nucleic acid sequence, and the method further comprises: demultiplexing the first and second pluralities of sequence reads based at least in part on detection of the second adaptor nucleic acid sequence in the second plurality of sequence reads.

Embodiment 125. The non-transitory computer readable storage medium of embodiment 124, wherein the second adaptor nucleic acid is attached onto 3′ end(s) of at least a portion of the second nucleic acid molecules of the plurality.

Embodiment 126. The non-transitory computer readable storage medium of embodiment 124, wherein the second adaptor nucleic acid is attached onto 5′ end(s) of at least a portion of the second nucleic acid molecules of the plurality.

Embodiment 127. The non-transitory computer readable storage medium of embodiment 124, wherein the second adaptor nucleic acid is between 5′ and 3′ ends of at least a portion of the second nucleic acid molecules of the plurality.

Embodiment 128. The non-transitory computer readable storage medium of any one of embodiments 117-127, wherein the first plurality of sequence reads is at least 2×, at least 4×, at least 8×, at least 10×, at least 50×, at least 100×, or at least 1000× more abundant than the second plurality of sequence reads.

Embodiment 129. The non-transitory computer readable storage medium of any one of embodiments 117-127, wherein the second plurality of sequence reads is at least 2×, at least 4×, at least 8×, at least 10×, at least 50×, at least 100×, or at least 1000× more abundant than the first plurality of sequence reads.

Embodiment 130. The non-transitory computer readable storage medium of any one of embodiments 117-129, wherein the first nucleic acid molecules are obtained from a sample prior to cytosine conversion.

Embodiment 131. The non-transitory computer readable storage medium of embodiment 130, wherein the sample is from an individual having or suspected of having a cancer.

Embodiment 132. The non-transitory computer readable storage medium of embodiment 131, wherein the sample comprises tissue, cells, and/or nucleic acids from a cancer.

Embodiment 133. The non-transitory computer readable storage medium of embodiment 131 or embodiment 132, wherein the sample comprises tissue, cells, and/or nucleic acids from normal tissue.

Embodiment 134. The non-transitory computer readable storage medium of any one of embodiments 130-133, wherein the first and/or second plurality of sequence reads is obtained by sequencing; optionally wherein the sequencing comprises use of a massively parallel sequencing (MPS) technique, whole genome sequencing (WGS), whole exome sequencing, targeted sequencing, direct sequencing, or a Sanger sequencing technique; and optionally wherein the massively parallel sequencing technique comprises next generation sequencing (NGS).

Embodiment 135. The non-transitory computer readable storage medium of any one of embodiments 130-134, wherein the method further comprises generating, based at least in part on the analyzing, a molecular profile for the sample.

Embodiment 136. The non-transitory computer readable storage medium of embodiment 135, wherein a treatment is administered to an individual based at least in part on the molecular profile.

Embodiment 137. The non-transitory computer readable storage medium of embodiment 135 or embodiment 136, wherein the molecular profile further comprises results from a comprehensive genomic profiling (CGP) test, a gene expression profiling test, a cancer hotspot panel test, a DNA methylation test, a DNA fragmentation test, an RNA fragmentation test, or any combination thereof.

Embodiment 138. The non-transitory computer readable storage medium of any one of embodiments 135-137, wherein the molecular profile further comprises results from a nucleic acid sequencing-based test.

Embodiment 139. The non-transitory computer readable storage medium of any one of embodiments 117-138, wherein the method further comprises comparing a sequence read of the second plurality of sequence reads with a sequence read of the first plurality of sequence reads.

Embodiment 140. The non-transitory computer readable storage medium of any one of embodiments 117-138, wherein the method further comprises comparing one or more sequence read(s) of the first and/or second plurality of sequence reads with a reference genome sequence.

Embodiment 141. A method of detecting genetic and epigenetic information in a single workflow, comprising:
providing a plurality of first single-stranded DNA fragments;
subjecting the plurality of first single-stranded DNA fragments to a round of primer extension in the presence of: (a) a primer that anneals to at least a portion of the first single-stranded DNA fragments, (b) a nucleic acid polymerase, and (c) a mixture of nucleotides comprising a cytosine analog that is resistant to TET-assisted pyridine borane treatment, thereby generating a plurality of first strands corresponding to the first single-stranded DNA fragments and a plurality of second strands that are complementary to the first strands, wherein the second strands comprise the cytosine analog that is resistant to TET-assisted pyridine borane treatment;
subjecting the pluralities of the first and second strands to TET-assisted pyridine borane treatment under conditions such that any methylated cytosines if present in the first strands undergo cytosine conversion; and
detecting at least a portion of the plurality of first strands and at least a portion of the plurality of second strands.

Embodiment 142. A method of detecting genetic and epigenetic information in a single workflow, comprising:
providing a plurality of first single-stranded DNA fragments;
attaching a first adaptor nucleic acid onto at least a portion of the first single-stranded DNA fragments of the plurality;
subjecting the plurality of first single-stranded DNA fragments to a round of primer extension in the presence of: (a) a primer that anneals to at least a portion of the first single-stranded DNA fragments downstream or at the first adaptor nucleic acid, (b) a nucleic acid polymerase, and (c) a mixture of nucleotides comprising a cytosine analog that is resistant to TET-assisted pyridine borane treatment, thereby generating a plurality of first strands comprising the first single-stranded DNA fragments with the first adaptor nucleic acid and a plurality of second strands comprising second single-stranded DNA fragments complementary to the first single-stranded DNA fragments and a second adaptor nucleic acid complementary to the first adaptor nucleic acid, wherein the second strands comprise the cytosine analog that is resistant to TET-assisted pyridine borane treatment;
subjecting the pluralities of the first and second strands to TET-assisted pyridine borane treatment under conditions such that any methylated cytosines if present in the first strands undergo cytosine conversion, wherein after cytosine conversion, the sequences of the first and second adaptor nucleic acids are no longer complementary; and
detecting at least a portion of the plurality of first strands and at least a portion of the plurality of second strands.

Embodiment 143. A method of detecting genetic and epigenetic information in a single workflow, comprising:
providing a plurality of first single-stranded DNA fragments;
subjecting the plurality of first single-stranded DNA fragments to a round of primer extension in the presence of: (a) a primer comprising: (i) a second adaptor nucleic acid portion comprising a non-complementary 5′ overhang that does not anneal to the first single-stranded DNA fragments, and (ii) a portion that anneals to at least a portion of the first single-stranded DNA fragments, (b) a nucleic acid polymerase, and (c) a mixture of nucleotides comprising a cytosine analog that is resistant to TET-assisted pyridine borane treatment, thereby generating a plurality of first strands comprising the first single-stranded DNA fragments with a first adaptor nucleic acid complementary to the portion of the second adaptor nucleic acid that anneals to the first single-stranded DNA fragments and a plurality of second strands comprising second single-stranded DNA fragments complementary to the first single-stranded DNA fragments and the second adaptor nucleic acid, wherein the second strands comprise the cytosine analog that is resistant to TET-assisted pyridine borane treatment;
subjecting the pluralities of the first and second strands to TET-assisted pyridine borane treatment under conditions such that any methylated cytosines if present in the first strands undergo cytosine conversion; and
detecting at least a portion of the plurality of first strands and at least a portion of the plurality of second strands.

Embodiment 144. A method of detecting genetic and epigenetic information in a single workflow, comprising:
providing a plurality of first single-stranded DNA fragments;
subjecting the plurality of first single-stranded DNA fragments to a round of primer extension in the presence of: (a) a primer comprising: (i) a second adaptor nucleic acid portion that does not anneal to the first single-stranded DNA fragments and (ii) a portion that anneals to at least a portion of the first single-stranded DNA fragments, (b) a nucleic acid polymerase, and (c) a mixture of nucleotides comprising a cytosine analog that is resistant to TET-assisted pyridine borane treatment, thereby generating a plurality of first strands comprising the first single-stranded DNA fragments with a first adaptor nucleic acid complementary to the second adaptor nucleic acid and a plurality of second strands comprising second single-stranded DNA fragments complementary to the first single-stranded DNA fragments and the second adaptor nucleic acid non-complementary to the first adaptor nucleic acid, wherein the second strands comprise the cytosine analog that is resistant to TET-assisted pyridine borane treatment;
subjecting the pluralities of the first and second strands to TET-assisted pyridine borane treatment under conditions such that any methylated cytosines if present in the first strands undergo cytosine conversion, wherein after cytosine conversion, the sequences of the first and second adaptor nucleic acids are no longer complementary; and
detecting at least a portion of the plurality of first strands and at least a portion of the plurality of second strands.

The disclosures of all publications, patents, and patent applications referred to herein are each hereby incorporated by reference in their entireties. To the extent that any reference incorporated by reference conflicts with the instant disclosure, the instant disclosure shall control.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Proof-of-Concept Demonstration of Genetic and Epigenetic Information in a Single Workflow

This Example provides proof-of-concept analysis of genetic (e.g., sequence) and epigenetic (e.g., methylation) information in a single workflow. In particular, this Example demonstrates that the “genomic strand” (for sequence information) can be synthesized from the original DNA at high efficiency and can be protected from cytosine conversion.

### Materials and Methods

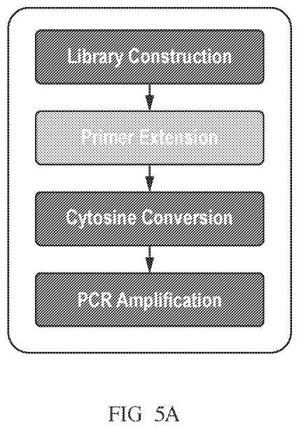

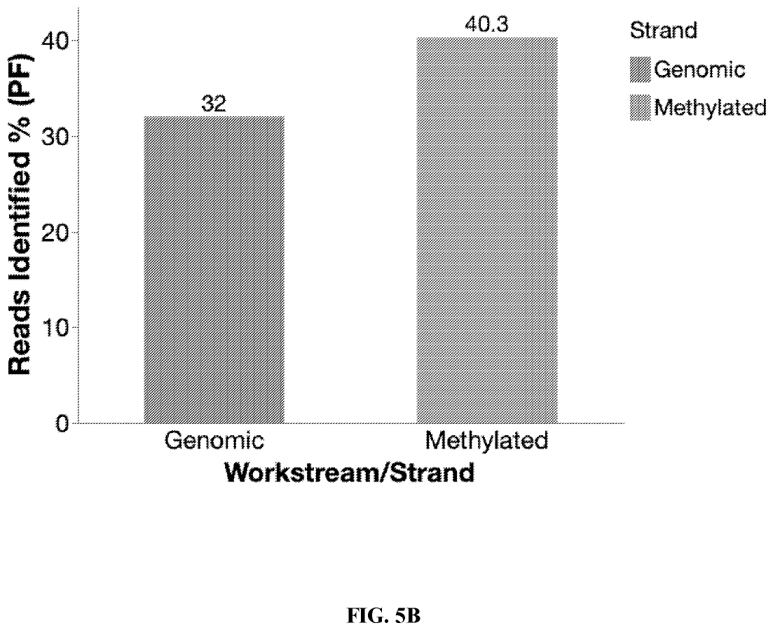

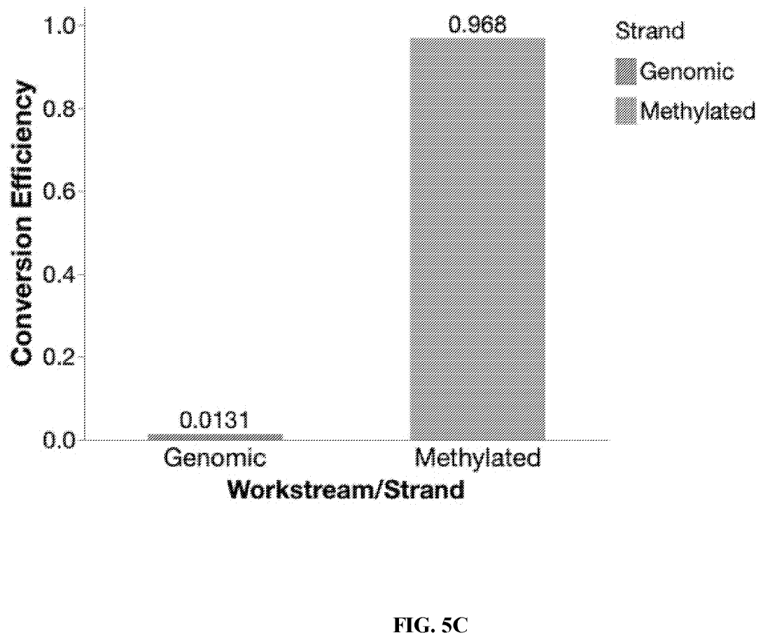

Genomic libraries were prepared from 20 ng of methylated and non-methylated HCT116 human cell lines using NEBNext Ultra II library preparation kit following standard procedures. Full-length, methylated adapters containing the 4 base “work-stream” tag (GGCC) between the P7 sequence and i7 index were ligated to the DNA. A round of primer extension was performed using NEB Q5 polymerase and Zymo dNTP mix including 5-methyl-dCTP, followed by enzymatic cytosine conversion using the NEBNext® Enzymatic Methyl-seq Kit. Finally, the converted libraries are PCR amplified with NEB Q5U polymerase and P5/P7 primers. After PCR amplification, samples were normalized to 1 nM and subjected to NGS sequencing on the Novaseq, achieving a mean coverage of 52×. An overview of the workflow used is provided in FIG. 5A.

### Results

After the workflow shown in FIG. 5A, the proportions of the “genomic strand” (e.g., incorporating the conversion-resistant cytosine analog) and its amplification products, and the “methylation strand” (e.g., without the cytosine analog and containing any DNA methylation present) and its amplification products were determined by percentage of reads identified. Reads were demultiplexed using a 4-bp workstream index to distinguish the genomic and methylation strands and their amplification products as a result of cytosine conversion.

As shown in FIG. 5B, both genomic and methylation strands were identified via sequencing based on the sequence reads identified from each. The genomic strand was synthesized via the primer extension step at an efficiency of 80%, thereby demonstrating that this strand was synthesized at high efficiency during primer extension.

FIG. 5C shows the results of cytosine conversion. The genomic strand was largely preserved after cytosine conversion with a protection efficiency of ˜98.7%. In contrast, the methylation strand was converted at an efficiency of ˜96.8%. These results demonstrate that the genomic strand and its amplification products were protected from cytosine conversion, thereby preserving sequence information.

### Example 2: Detection of Genetic and Epigenetic Variants in Cancer cfDNA in a Single Assay

This Example demonstrates that cancer associated methylation and genetic variants are detectable in cfDNA using single workflow methodology. Furthermore, the single workflow from the same input DNA input mass can produce the equivalent signals relative to stand-alone methods (e.g., WGS and WG methylation sequencing).

### Materials and Methods

4 cancer cfDNA samples with characterized genetic variants and 2 non-cancerous cfDNA samples were analyzed. Using an input of 20 ng cfDNA, each sample was tested in the following assays: standard WGS, standard WG methylation (using enzymatic methylation sequencing), and the single workflow protocol. The standard WGS libraries were generated using the NEBNext Ultra II library preparation kit following standard procedures and PCR amplified using NEB Q5 polymerase and NEB Unique Dual Index Primers. The standard WG methylation libraries were prepared using NEBNext® Enzymatic Methyl-seq Kit following standard procedures and PCR amplified with NEB Q5U polymerase and NEB Unique Dual Index Primers. Single Workflow libraries were prepared using the protocol described herein (using enzymatic methylation sequencing for cytosine conversion and 5-mC for the cytosine analog), as described in Example 1. After PCR amplification, libraries were normalized to 1 nM and subjected to NGS sequencing on the Novaseq, achieving an average coverage of 109×.

### Results

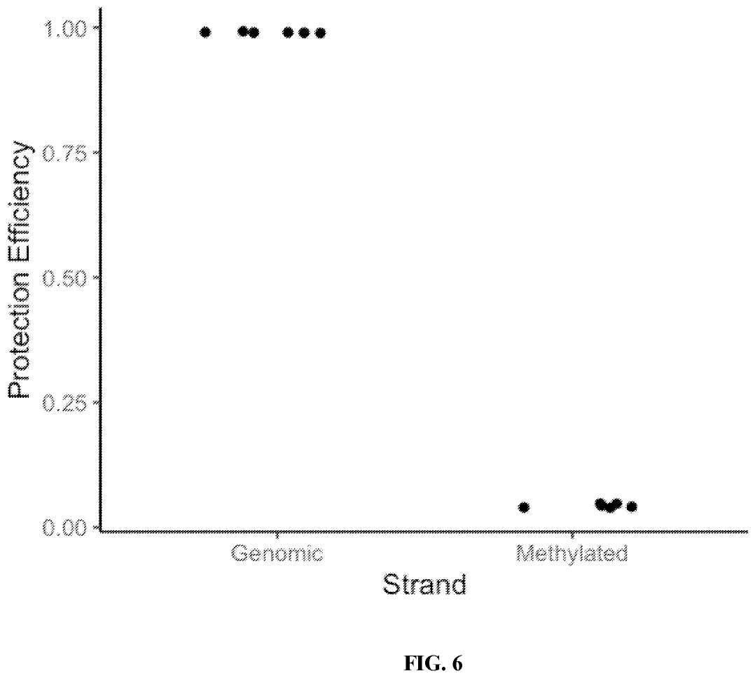

First, protection efficiency between genomic and methylation strands in single workflow from cfDNA was assessed. The genomic strand refers to a cytosine conversion protected copy of the original molecule generated with 5-mC. As shown in FIG. 6, cytosines were 99.05% protected from enzymatic conversion (mean derived from 6 libraries), allowing the preservation of genetic information. The methylated strand was converted and contains methylation information, as demonstrated with a protection efficiency of 4.3% (mean derived from 6 libraries).

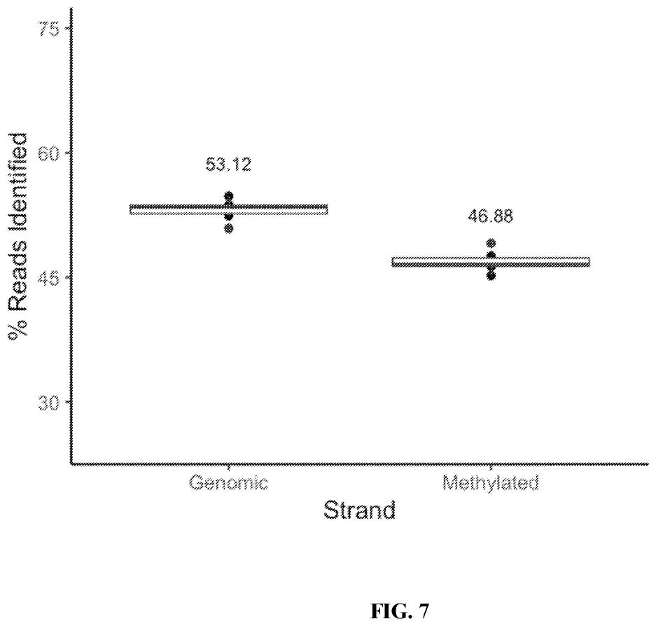

Next, synthesis of the genomic strand was evaluated. Across 6 samples, the average % Reads Identified for genomic and methylated strand were 53.12 and 46.88 respectively (FIG. 7). These results demonstrate that primer extension is highly efficient in the synthesis of the genomic strand from the original DNA molecule. Reads in the library were identified with the 4 base workstream index that distinguishes genomic and methylated strands and their amplification products.

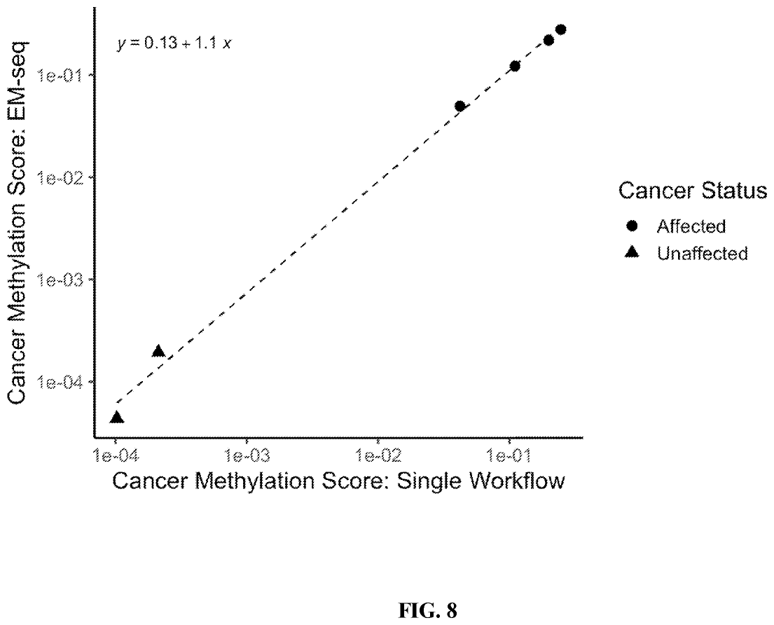

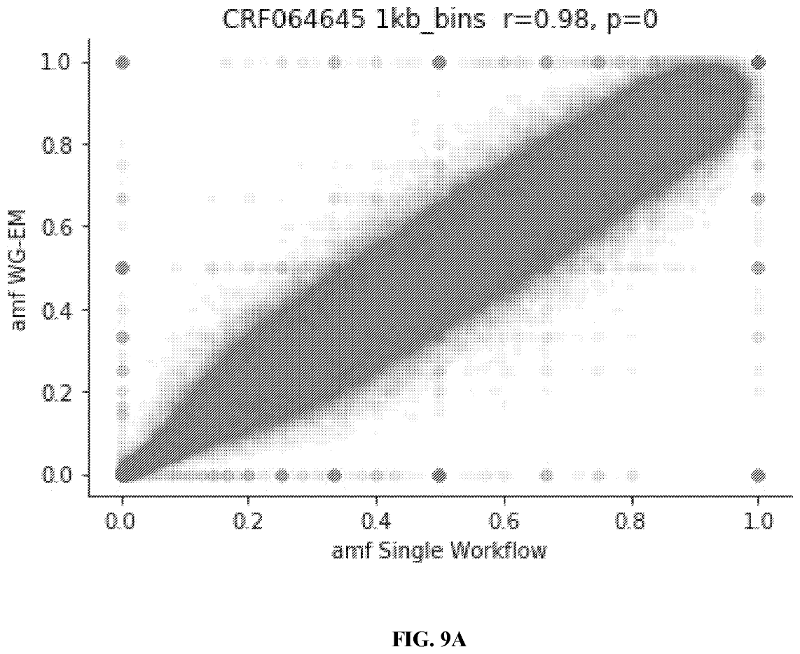

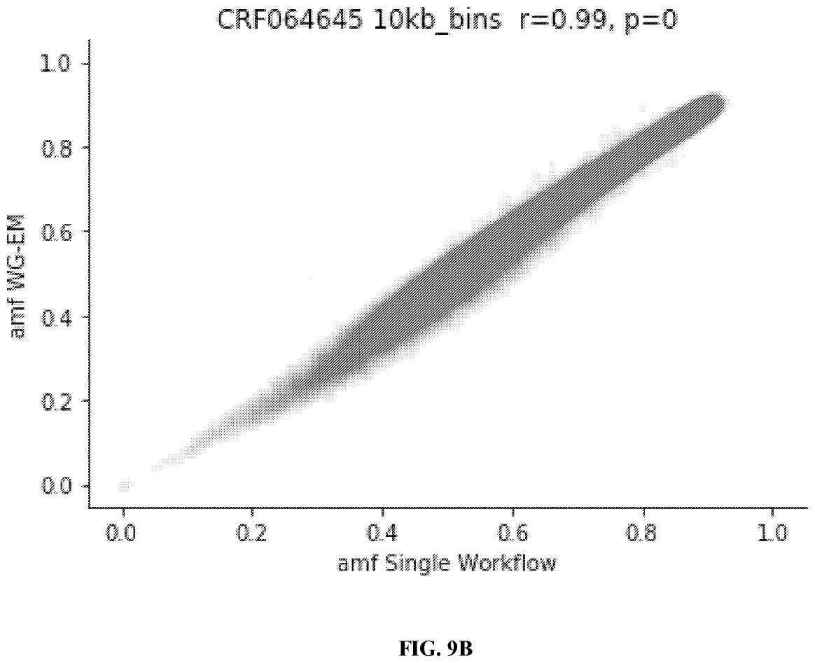

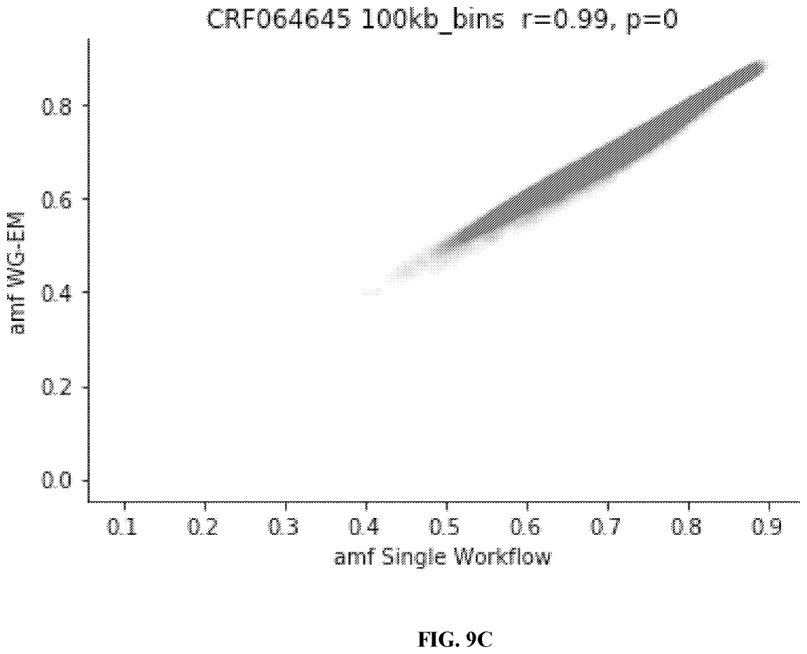

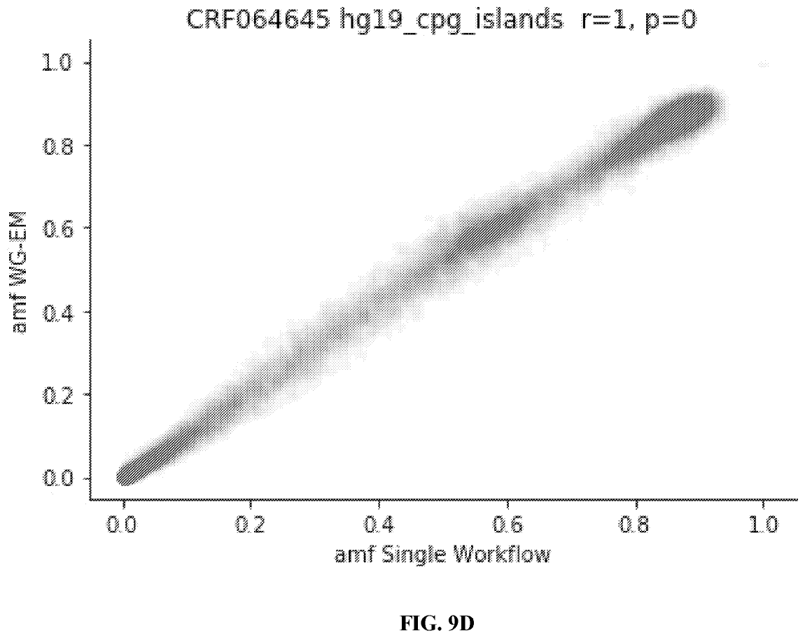

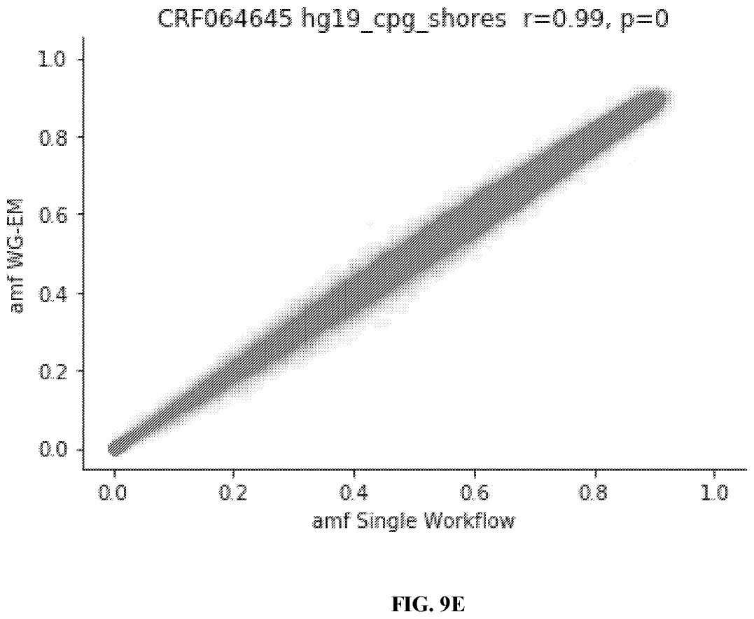

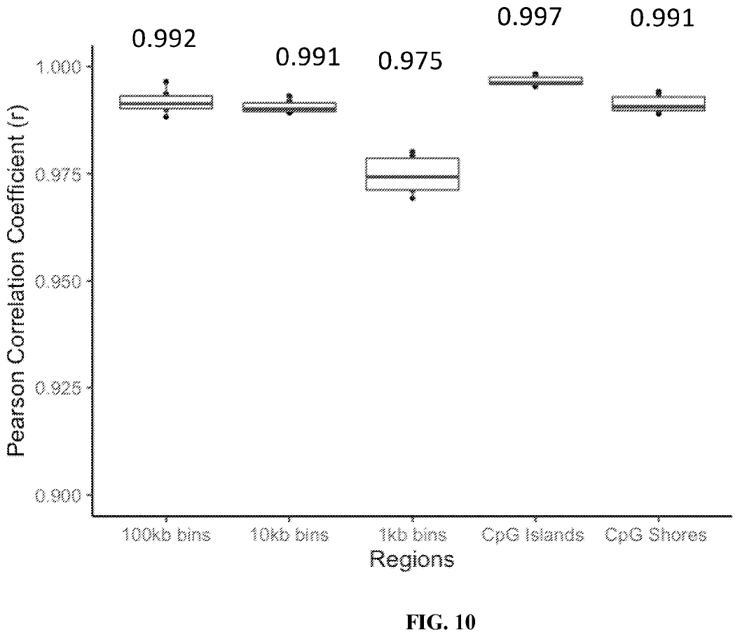

Methylation of the cancer cfDNA samples was analyzed via WG enzymatic methylation sequencing compared with the single workflow methodology. Cancer Methylation Score, which assesses consensus methylated sites from individual DNA molecules, was strongly correlated in single workflow and WG enzymatic methylation sequencing methods, as shown in FIG. 8. The best fit line had a slope of 1.1 and R2=1. Higher correlations in methylation levels were observed between the single workflow and standard WG methylation methodologies in smaller bins and functional regions, including 1 kb bins (FIG. 9A), 10 kb bins (FIG. 9B), 100 kb bins (FIG. 9C), CpG islands (FIG. 9D), and CpG shores (FIG. 9E). Sex chromosomes were not included. No filtering was applied to bins. Average methylation fraction was also preserved in the single workflow technique vs. standard WG methylation analysis. Pearson Correlation Coefficient of average methylation fraction (AMF) in single workflow compared to the standard WG enzymatic methylation sequencing is shown in FIG. 10. These results demonstrate high concordance between methodologies, with an r above 0.975 in smaller bin sizes and in functional regions. These results demonstrate that methylation analysis using the single workflow technique was highly concordant with methylation analysis obtained using standard techniques such as WG enzymatic methylation sequencing.

Next, analysis of genetic variants using single workflow was characterized. Table A shows variants analyzed.

TABLE A
Genetic variants analyzed via single workflow and standard WGS.

| Chromosome | Position | Gene | Participant ID |
|---|---|---|---|
| chr5 | 112175639 | APC | CRF059256 |
| chr5 | 112174696 | APC | CRF059256 |
| chr12 | 25398284 | KRAS | CRF059256 |
| chr17 | 29679408 | NF1 | CRF064005 |
| chr16 | 68846037 | CDH1 | CRF064005 |
| chr14 | 105246551 | AKT1 | CRF064005 |
| chr12 | 25398284 | KRAS | CRF064645 |
| chr13 | 32953592 | BRCA2 | CRF064645 |
| chr5 | 112175162 | APC | CRF064645 |
| chr17 | 7577498 | TP53 | CRF064645 |
| chr5 | 112175303 | APC | US0756490 |
| chr17 | 7578265 | TP53 | US0756490 |
| chr5 | 112116592 | APC | US0756490 |
| chr7 | 140453154 | BRAF | US0756490 |
| chr3 | 178916854 | PIK3CA | US0756490 |
| chr13 | 32914413 | BRCA2 | US0756490 |

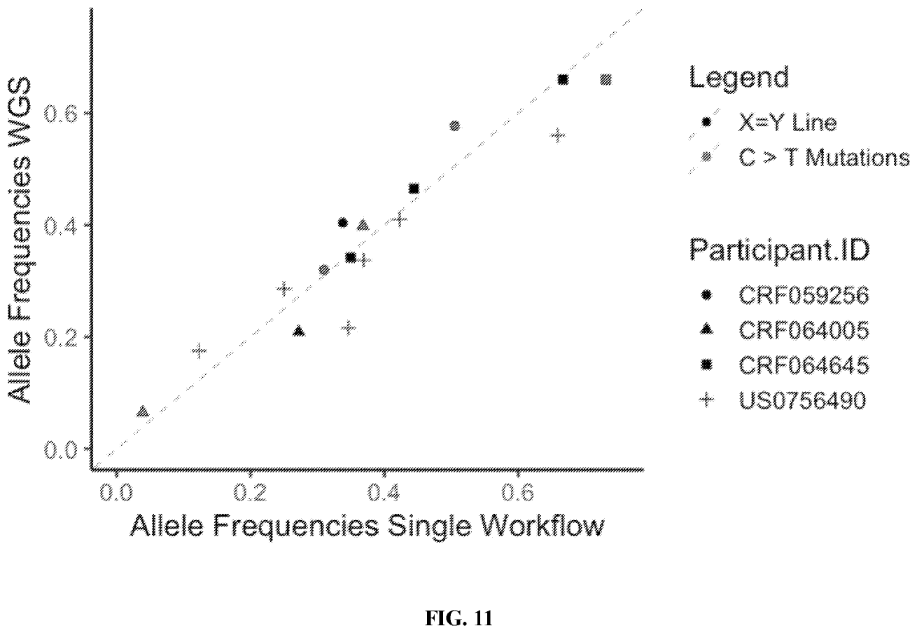

High concordance in called allele frequencies was observed between single workflow and WGS in the 4 cancer cfDNA samples (FIG. 11). 16 characterized variants with >3% allele frequencies (AF) were shared between single workflow and standard WG techniques. These results demonstrate that genomic signal from the single workflow methodology was preserved, with strong concordance in detection of genetic variants.

In conclusion, single workflow methods allow the multi-omic detection of methylation and genetic information in one simplified workflow with limited input DNA needed. The results from this Example demonstrated that the assay works on a technical level in cfDNA. The genomic strand was synthesized from the original/methylated DNA at a high efficiency and protected during cytosine conversion. The signals were also preserved in single workflow compared to the standard, stand-alone methods, as demonstrated by strong correlations in both cancer associated methylation scores and genetic variant calling. The single workflow assay also allowed for C->T variant calling despite the genomic strand exposure to cytosine conversion conditions.

### Example 3: Enrichment of Genomic and/or Methylation Strand Information in Single Workflow Assays

This Example demonstrates that the genomic and methylation strands can be amplified during the single workflow assay by using a primer designed to specifically bind to the genomic or methylation strand during PCR amplification.

### Materials and Methods

20 ng input DNA was used from methylated and non-methylated HCT116 human cell lines. Single workflow analysis was carried out as described in Examples 1 and 2. Two primer conditions were tested by PCR: (1) standard single workflow primers as described above, and (2) a primer designed to specifically bind to the genomic strand and P5 primer. Abundance of genomic vs. methylated strands was determined using % of sequence reads as described above. Subsequent assays used a primer designed to specifically bind to the methylation strand and P5 primer as well. The sequences used for genomic strand specific primer is CAA GCA GAA GAC GGC ATA CGA GAT TT (SEQ ID NO:1). The sequence used for methylation strand specific primer is CAA GCA GAA GAC GGC ATA CGA GAT CC (SEQ ID NO:2).

### Results

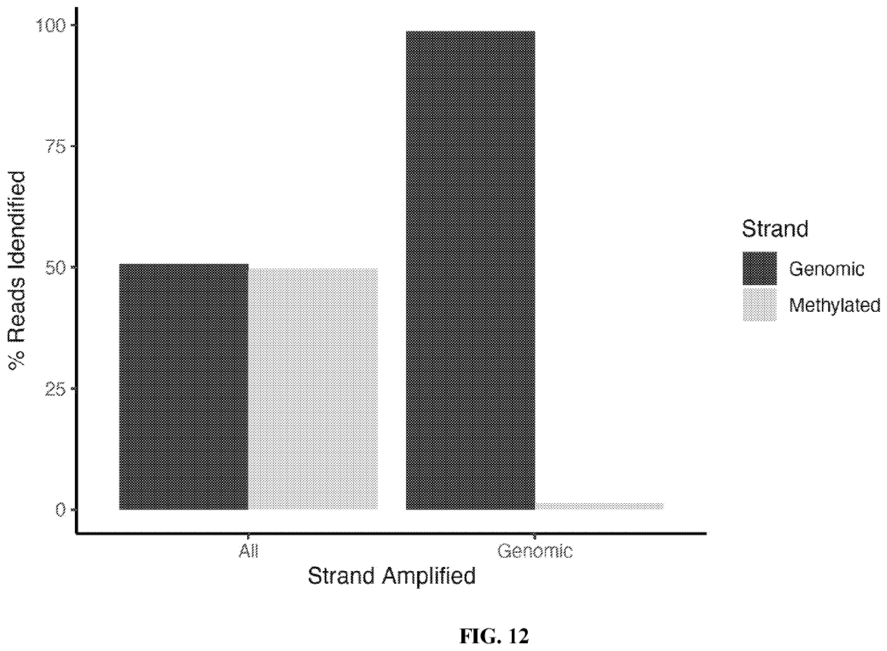

Preferential amplification of the genomic strand was achieved using the genomic strand-specific primers. Across 2 samples, the average % reads of the genomic strand was 98.7% and 1.3% of the methylated strand, as shown in FIG. 12. However, using the standard single workflow primers, the average % reads of the genomic strand was 50.5% and 49.5% of the methylated strand.

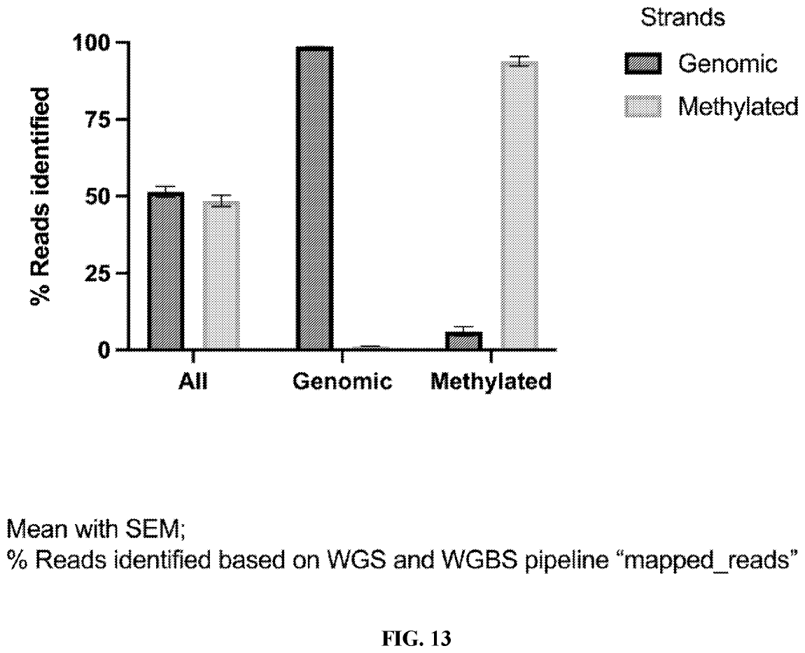

Using primers specific for either the genomic or the methylation strand, preferential amplification and enrichment of either strand can be achieved. FIG. 13 shows that this strategy was successfully used to selectively amplify either strand. As such, specific primers can be used to amplify either or both genomic and methylation strands, e.g., for enrichment prior to sequencing.

This Example demonstrates that genomic strands and methylation strands can be jointly captured from a single workflow library with the inclusion of hybrid capture baits complimentary to genomic regions of interest and capture baits complimentary to cytosine converted methylation regions of interest.

Custom hybrid capture baits targeting actionable mutations in the genomic strand were obtained (2.2 Mb). A distinct set of custom baits complimentary to cytosine converted methylation biomarkers of interest were separately obtained (5.52 Mb). The genomic panel was mixed in an equimolar ratio with the custom methylation panel to create a Single workflow hybrid capture panel. 10 ng of cfDNA, isolated from a healthy donor, was input into Single workflow library preparation as described in Examples 1 and 2. 500 ng of resulting libraries were captured with the custom Single workflow panel using a capture kit.

After sequencing, the captured libraries were aligned to their respective genomic or methylation genomes, fragments were deduplicated, by fragment start/end position, and the average unique coverage over the baited regions were quantified. High unique coverage over targeted genomic (1598.9×) and methylation regions (1510.5×) was observed, as depicted in FIG. 14A. Given the low cfDNA input mass, this result demonstrates high capture efficiency of both strands (˜50%, given 3030 input haploid genomic equivalents) in joint capture from Single workflow libraries. The captures also showed high on-target rate of both genomic and methylation regions of interest, and even coverage across the distinct probe sets as measured by Fold-80 base penalty in FIG. 14B.

1. A method of detecting genetic and epigenetic information in a single workflow, comprising:
providing a plurality of first single-stranded DNA fragments;
subjecting the plurality of first single-stranded DNA fragments to a round of primer extension in the presence of: (a) a primer that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands corresponding to the first single-stranded DNA fragments and a plurality of second strands that are complementary to the first strands, wherein the second strands comprise the cytosine analog that is resistant to cytosine conversion;
subjecting the pluralities of the first and second strands to a cytosine conversion treatment under conditions such that unmethylated cytosine(s) if present in the first strands undergo cytosine conversion; and
detecting methylation information from at least a portion of the plurality of first strands and detecting sequence information from at least a portion of the plurality of second strands.

2. The method of claim 1, wherein the detecting is by sequencing, microarray, quantitative PCR (qPCR), digital droplet PCR (ddPCR), next-generation sequencing (NGS), or molecular inversion probes.

3-4. (canceled)

5. The method of claim 1, wherein the method further comprises, prior to detecting: subjecting the plurality of first strands and/or the plurality of second strands to amplification, wherein the detecting comprises detecting at least a portion of the pluralities of first and second strands and their amplification products if present.

6. The method of claim 5, comprising:
(a) subjecting the plurality of second strands to amplification in the presence of one or more primer(s) that anneal with at least a portion of the plurality of second strands, but not with the plurality of first strands; and/or
(b) subjecting the plurality of first strands to amplification in the presence of one or more primer(s) that anneal with at least a portion of the plurality of first strands, but not with the plurality of second strands.

7-10. (canceled)

11. The method of claim 1, wherein the method further comprises, prior to detecting: enriching for the plurality of second strands or their amplification products; and/or enriching for the plurality of first strands or their amplification products.

12. (canceled)

13. The method of claim 11, wherein the method comprises, prior to detecting: separating one or more first strands or their amplification products from one or more second strands or their amplification products.

14. The method of claim 13, wherein the separation comprises:
(1) (a) combining one or more bait molecules with the pluralities of first and second strands, wherein the one or more bait molecules preferentially hybridize with one or more of the first strands or their amplification products, or with one or more of the second strands or their amplification products, thereby producing nucleic acid hybrids; and (b) isolating the nucleic acid hybrids; or
(2) (a) combining one or more first bait molecules with the pluralities of first and second strands, wherein the one or more first bait molecules preferentially hybridize with one or more of the first strands or their amplification products, thereby producing first nucleic acid hybrids; (b) isolating the first nucleic acid hybrids; (c) combining one or more second bait molecules with the pluralities of first and second strands, wherein the one or more second bait molecules preferentially hybridize with one or more of the second strands or their amplification products, thereby producing second nucleic acid hybrids; and (d) isolating the second nucleic acid hybrids.

15-20. (canceled)

21. The method of claim 1, wherein the method comprises subjecting the plurality of first single-stranded DNA fragments to two or more rounds of primer extension prior to cytosine conversion in the presence of: (a) a primer that anneals to at least a portion of the first single-stranded DNA fragments, (b) a nucleic acid polymerase, and (c) a mixture of nucleotides comprising a cytosine analog that is resistant to cytosine conversion.

22. The method of claim 1, further comprising attaching one or more nucleic acid adaptors to one or more of the first single-stranded DNA fragments and/or to one or more of the second strands.

23-24. (canceled)

25. A method of detecting genetic and epigenetic information in a single workflow, comprising:
providing a plurality of first single-stranded DNA fragments;
attaching a first adaptor nucleic acid onto at least a portion of the first single-stranded DNA fragments of the plurality;
subjecting the plurality of first single-stranded DNA fragments to a round of primer extension in the presence of: (a) a primer that anneals to at least a portion of the first single-stranded DNA fragments downstream or at the first adaptor nucleic acid, (b) a nucleic acid polymerase, and (c) a mixture of nucleotides comprising a cytosine analog that is resistant to cytosine conversion, thereby generating a plurality of first strands comprising the first single-stranded DNA fragments with the first adaptor nucleic acid and a plurality of second strands comprising second single-stranded DNA fragments complementary to the first single-stranded DNA fragments and a second adaptor nucleic acid complementary to the first adaptor nucleic acid, wherein the second strands comprise the cytosine analog that is resistant to cytosine conversion;
subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and
detecting methylation information from at least a portion of the plurality of first strands and detecting sequence information from at least a portion of the plurality of second strands.

26-27. (canceled)

28. The method of claim 25, wherein the first adaptor nucleic acid comprises:
(a) one or more unmethylated cytosines that are converted during cytosine conversion; or
(b) one or more methylated cytosines, wherein the primer comprises one or more unmethylated cytosines that are converted during cytosine conversion.

29. (canceled)

30. A method of detecting genetic and epigenetic information in a single workflow, comprising:
providing a plurality of first single-stranded DNA fragments;
subjecting the plurality of first single-stranded DNA fragments to a round of primer extension in the presence of: (a) a primer comprising: (i) a second adaptor nucleic acid portion that does not anneal to the first single-stranded DNA fragments and (ii) a portion that anneals to at least a portion of the first single-stranded DNA fragments, (b) a nucleic acid polymerase, and (c) a mixture of nucleotides comprising a cytosine analog that is resistant to cytosine conversion, thereby generating a plurality of first strands comprising the first single-stranded DNA fragments with a first adaptor nucleic acid complementary to the second adaptor nucleic acid and a plurality of second strands comprising second single-stranded DNA fragments complementary to the first single-stranded DNA fragments and the second adaptor nucleic acid non-complementary to the first adaptor nucleic acid, wherein the second strands comprise the cytosine analog that is resistant to cytosine conversion;
subjecting the pluralities of the first and second strands to a cytosine conversion treatment under conditions such that unmethylated cytosine(s) if present in the first strands undergo cytosine conversion, wherein after cytosine conversion, the sequences of the first and second adaptor nucleic acids are no longer complementary; and
detecting methylation information from at least a portion of the plurality of first strands and detecting sequence information from at least a portion of the plurality of second strands.

31-32. (canceled)

33. The method of claim 30, wherein
the second adaptor nucleic acid portion of the primer comprises a non-complementary 5′ overhang that does not anneal to the first single-stranded DNA fragments, and the plurality of first strands comprise the first single-stranded DNA fragments with the first adaptor nucleic acid complementary to the portion of the second adaptor nucleic acid that anneals to the first single-stranded DNA fragments.

34-36. (canceled)

37. The method of claim 25, further comprising: demultiplexing sequence information from the first and second strands based on the first and/or second adaptor nucleic acids.

38. The method of claim 25, wherein the method further comprises, prior to detecting: subjecting the plurality of first strands and/or the plurality of second strands to amplification, wherein the detecting comprises detecting at least a portion of the pluralities of first and second strands and their amplification products if present.

39-59. (canceled)

60. The method of claim 1, wherein the plurality of first strands is sequenced at a different depth of sequencing than the plurality of second strands.

61-64. (canceled)

65. The method of claim 1, further comprising obtaining the plurality of single- or double-stranded DNA fragments from a sample, wherein the sample comprises tissue, cells, and/or nucleic acids from a cancer and/or from normal tissue.

66-73. (canceled)

74. The method of claim 1, wherein the cytosine analog that is resistant to cytosine conversion comprises 5-methylcytosine (5mC), 5-hydroxymethylcytosine (5hmC), 5-carboxylcytosine (5caC), 5-formylcytosine, 5-(beta-D-glucosylmethyl) cytosine (5gmC), 5-ethyl dCTP, 5-methyl dCTP, 5-fluoro dCTP, 5-bromo dCTP, 5-iodo dCTP, 5-chloro dCTP, 5-trifluoromethyl dCTP, or 5-aza dCTP.

75. The method of claim 1, wherein the cytosine conversion is by bisulfite treatment, TET-assisted bisulfite treatment, oxidative bisulfite treatment, APOBEC, or TET/beta-glucosyltransferase assisted APOBEC treatment.

76-79. (canceled)

80. The method of claim 1, wherein the nucleic acid polymerase is capable of incorporating the cytosine analog into nucleic acid.

81-144. (canceled)

145. The method of claim 1, wherein the plurality of first single-stranded DNA fragments is from a sample obtained from an individual.

146. The method of claim 145, comprising:
based on the detected methylation information, determining a methylation level of one or more genes in the sample;
based on the sequence information, detecting one or more mutations in one or more genes in the sample; and
based at least in part on the methylation level and/or mutation(s) determined in the sample, determining whether the individual has or does not have cancer.

147. The method of claim 145, comprising:
based on the detected methylation information, determining a methylation level of one or more genes in the sample;
based on the sequence information, detecting one or more mutations in one or more genes in the sample; and
based at least in part on the methylation level and/or mutation(s) determined in the sample, detecting a response to a treatment by the individual.

148. The method of claim 145, comprising:
based on the detected methylation information, determining a methylation level of one or more genes in the sample;
based on the sequence information, detecting one or more mutations in one or more genes in the sample; and
based at least in part on the methylation level and/or mutation(s) determined in the sample, detecting minimal residual disease or a lack thereof in the sample.

149. The method of claim 146, wherein the one or more mutations are somatic mutations.

150. The method of claim 146, wherein the one or more mutations are germline mutations.

151. The method of claim 146, comprising, based on the sequence information, detecting loss of heterozygosity of one or more HLA genes or tumor mutational burden.


SEQUENCE LISTING (4 sequences)

SEQ ID NO: 1 - DNA, 26 bp, synthetic construct
  source 1..26: mol_type = other DNA
caagcagaagacggcatacgagattt

SEQ ID NO: 2 - DNA, 26 bp, synthetic construct
  source 1..26: mol_type = other DNA
caagcagaagacggcatacgagatcc

SEQ ID NO: 3 - DNA, 12 bp, synthetic construct
  source 1..12: mol_type = other DNA
ctgatcgtggtt

SEQ ID NO: 4 - DNA, 12 bp, synthetic construct
  source 1..12: mol_type = other DNA
ctgatcgtggcc